(19) United States
(12) Patent Application Publication
(10) Pub. No.: US 2023/0065745 A1
(43) Pub. Date: Mar. 2, 2023

(54) PIPERIDINE-2,6-DIONE DERIVATIVES WHICH BIND TO CEREBLON, AND METHODS OF USE THEREOF

(71) Applicant: Captor Therapeutics S.A., Wroclaw (PL)

(72) Inventors: Katarzyna KACZANOWSKA, Wroclaw (PL); Sylvain COTTENS, Witterswil (CH); Roman PLUTA, Krakow (PL); Niall DICKINSON, Skierniewice (PL); Michal WALCZAK, Wroclaw (PL)

(21) Appl. No.: 17/780,894

(22) PCT Filed: Nov. 27, 2020
(86) PCT No.: PCT/EP2020/083597

§ 371 Date: May 27, 2022

(30) Foreign Application Priority Data
Nov. 27, 2019 (PL) PCT/PL2019/000108

(51) Int. Cl. A61K 31/4535; C07D 409/12; C07D 417/12; C07D 409/14; C07D 405/12; C07D 413/12; C07D 495/04; C07D 471/04; A61K 31/454; A61K 31/4525; A61K 31/4545; A61K 31/69; A61K 31/573; A61K 39/395

(52) U.S. Cl. CPC A61K 31/4535; C07D 409/12; C07D 417/12; C07D 409/14; C07D 405/12; C07D 413/12; C07D 495/04; C07D 471/04; A61K 31/454; A61K 31/4525; A61K 31/4545; A61K 31/69; A61K 31/573; A61K 39/3955

(57) ABSTRACT

The present invention provides novel compounds which bind to cereblon, and methods of use thereof. The compounds are represented by Formulas (Ia), (Ib), (IIa) and (IIb), below: Formula (Ia), Formula (Ib), Formula (IIa), Formula (IIb).

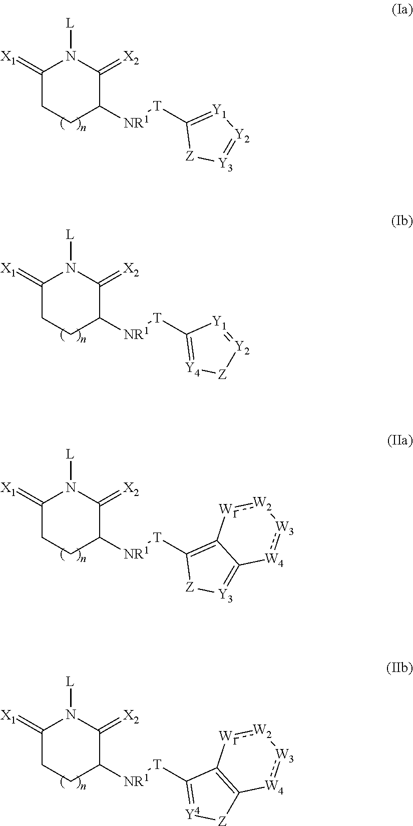

### FIELD OF THE INVENTION

The present invention relates to novel compounds which bind to the protein cereblon and modulate the substrate specificity of CUL4-DDB1-RBX1-CRBN ubiquitin ligase complex (CRL4^{CRBN}). Cereblon is a substrate recognition component of CRL4^{CRBN}. Chemical modulation of cereblon may induce association of novel substrate proteins, followed by their ubiquitination and degradation.

### BACKGROUND

Cereblon (CRBN) is a protein which associates with DDB1 (damaged DNA binding protein 1), CUL4 (Cullin-4), and RBX1 (RING-Box Protein 1). Collectively, the proteins form a ubiquitin ligase complex, which belongs to Cullin RING Ligase (CRL) protein family and is referred to as CRL4^{CRBN}. Cereblon became of particular interest to the scientific community after it was confirmed to be a direct protein target of thalidomide, which mediates the biological activity of cereblon. Thalidomide, a drug approved for treatment of multiple myeloma in the late 1990s, binds to cereblon and modulates the substrate specificity of the CRL4^{CRBN} ubiquitin ligase complex. This mechanism underlies the pleiotropic effect of thalidomide on both immune cells and cancer cells (see Lu G et al.: The Myeloma Drug Lenalidomide Promotes the Cereblon-Dependent Destruction of Ikaros Proteins. Science. 2014 Jan. 17; 343(6168): 305-9).

Thalidomide's success in cancer therapy stimulated efforts towards development of analogues with higher potency and fewer detrimental side effects. As a results, various drug candidates were produced: lenalidomide, pomalidomide, CC-220, CC-122, CC-885, and TD-106. These compounds are collectively called Cereblon Modulating Agents (CMAs). For discussions of these compounds, see—for example—U.S. Pat. No. 5,635,517 (B2), WO2008039489 (A2), WO2017197055 (A1), WO2018237026 (A1), WO2017197051 (A1), U.S. Pat. No. 8,518,972 (B2), EP 2057143 (B1), WO2019014100 (A1), WO2004103274 (A2), and Kim S A et al.: A novel cereblon modulator for targeted protein degradation. Eur J Med Chem. 2019 Mar. 15; 166: 65-74.

The clinical applicability of CMAs in numerous hematologic malignancies, such as multiple myeloma, myelodysplastic syndromes lymphomas and leukemia, has been demonstrated (see Le Roy A et al.: Immunomodulatory Drugs Exert Anti-Leukemia Effects in Acute Myeloid Leukemia by Direct and Immunostimulatory Activities. Front Immunol. 2018; 9: 977).

The antitumor activity of cereblon modulators is mediated by:

1) inhibition of cancer cell proliferation and induction of apoptosis,
2) disruption of trophic support from tumor stroma,
3) stimulation of immune cells, resulting in proliferation of T-cells, cytokine production and activation of NK (natural killer) cells (see Le Roy A et al.: Immunomodulatory Drugs Exert Anti-Leukemia Effects in Acute Myeloid Leukemia by Direct and Immunostimulatory Activities. Front Immunol. 2018; 9: 977).

It has been demonstrated that chemically-modified thalidomide-based derivatives can significantly modify the substrate specificity of CRL4^{CRBN} ubiquitin ligase. Thus, it is desired to progress development of cereblon modulating agents in order to achieve desired substrate specificity in the CMA-bound CRL4^{CRBN} ubiquitin ligase complex (see Sievers Q L et al.: Defining the human C₂H₂ zinc finger degrome targeted by thalidomide analogues through CRBN. Science. 2018 Nov. 2; 362(6414)) to reach a desired safety profile. There is thus a continuing need to provide novel cereblon-binding compounds which have pharmaceutically relevant properties.

Chemically-modified thalidomide-based derivatives, such as pomalidomide and lenalidomide, induce degradation of various neosubstrates, such as IKZF1, IKZF3, and/or CK1α. While degradation of IKZF1 and IKZF3 might be beneficial in the treatment of some cancer types (such as multiple myeloma), it may also contribute to dose-limiting toxicity of those compounds. Side effects resulting from lenalidomide's activity include neutropenia, thrombocytopenia, and hemorrhagic disorders (see: Sun X et al. PROTACs: great opportunities for academia and industry. Signal Transduct Target Ther. 2019 Dec. 24; 4:64 and Stahl M, Zeidan A M: Lenalidomide Use in Myelodysplastic Syndromes: Insights Into the Biologic Mechanisms and Clinical Applications. Cancer. 2017 May 15; 123(10):1703-1713). On the contrary, degradation of CK1α contributes to lenalidomide's therapeutic efficacy in myelodysplastic syndromes (see: Krönke J et al.: Lenalidomide induces ubiquitination and degradation of CK1α in del(5q) MDS. Nature. 2015 Jul. 9; 523(7559): 183-188. doi:10.1038/nature14610). Thus, new chemically-modified thalidomide-based derivatives capable of CK1α degradation and having more selective profile may be particularly useful in the treatment of cancer.

### SUMMARY OF INVENTION

In accordance with a first aspect of the invention, there is provided a compound of Formula (Ia) or (Ib):

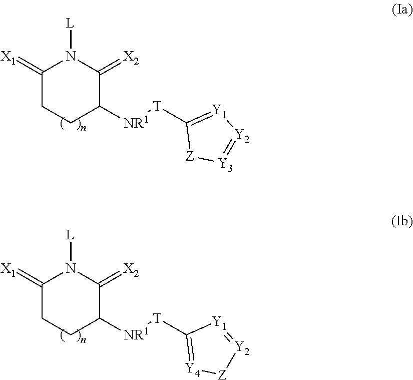

or a pharmaceutically acceptable salt or tautomer thereof,
wherein
each of X₁ and X₂ is independently O or S;
Z is O, S or NR²;
T is C═O or SO₂;
each of Y₁, Y₂, Y₃, and Y₄ is independently N or CR,
wherein at least one of Y₁, Y₂ and Y₃ in Formula (Ia) is CR, and at least one of Y₁, Y₂ and Y₄ in Formula (Ib) is CR;
n is 0, 1 or 2;
L is hydrogen, alkyl, alkenyl, aryl, heteroaryl, benzyl, haloalkyl, haloalkenyl, —C(O)R″, —CH₂C(O)OR″, —C(O)OR″, —C(O)NH₂, —C(O)NHR″, —C(O)NR″₂, —OR″, —NR″₂, or —S(O)₂R″;
each R is independently hydrogen, halogen, alkyl, cycloalkyl, heterocycloalkyl, alkenyl, cycloalkenyl, alkynyl, aryl, heteroaryl, benzyl, haloalkyl, haloalkenyl, —NH₂, —NHR″, —NR″₂, —CH₂NR″₂, —NR″C(O)R″, —NR″C(O)CH₂NR″₂, —NR″C(O)CH₂-heterocycloalkyl, —NR″C(O)CH(OH)R″, —CH₂NR″C(O)OR″, —NR″C(O)OR″, —NR″SO₂R″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —NHC(S)NHR″, SR″, or —S(O)₂R″, —S(O)₂OR″, —S(O)₂NH₂, —S(O)₂NHR″, or —S(O)₂NR″₂;
each R″ is independently hydrogen, alkyl, cycloalkyl, alkenyl, cycloalkenyl, aryl, heteroaryl, or benzyl;
R² is hydrogen, alkyl, cycloalkyl, alkenyl, cycloalkenyl, alkynyl, aryl, heteroaryl, benzyl, haloalkyl, haloalkenyl, —NH₂, —NHR″, —NR″₂, —NR″C(O)R″, —N[C(O)R″]₂, —NR″C(O)OR″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, or —S(O)₂R″, —S(O)₂OR″, —S(O)₂NH₂, —S(O)₂NHR″, or —S(O)₂NR″₂;
R¹ is hydrogen, alkyl, cycloalkyl, alkenyl, cycloalkenyl, aryl, heteroaryl, or benzyl;
wherein when Z is O, then Y₂ is CR′ and
   wherein when the compound is of Formula (Ia), then
(i) when each of Y₁, Y₂ and Y₃ is CR, then at least one of R is not H;
(ii) when Z is NR², then Y₂ and Y₃ are CR;
(iii) when Z is S, then Y₁ is not C—OMe and Y₂ is not C—OMe;
(iv) when Z is S and Y₁ is C—NHCOMe, then Y₃ is not C—CH₂NR″C(O)OR″;
(v) when Z is S and Y₁ is N, then Y₂ is not C—H, C-aryl or C—C(O)OR″; and
(vi) when Z is S and Y₂ is N, then Y₃ is C—NH₂, C—NHR″, C—NR″₂, C—NR″C(O)OR″, C—CH₂NR″C(O)OR″, C-haloalkyl, C-^{t}Butyl, C—OR″, C—COOR″ or C—SR″; wherein when Y₃ is C—NH₂, C—NHR″ or C—NR″₂, then Y₁ is C—H;
   and when the compound is of Formula (Ib), then:
(vii) when each of Y₁, Y₂ and Y₄ is CR, then at least one of R is not H;
(viii) when Z is S, then Y₁ is not C—COOH or C—NHC(O)Me, and Y₄ is not C—Br;
(ix) when Z is S and Y₂ is C—Br, then Y₄ is C—OR″
(x) when Z is S, Y₁ is N and Y₂ is C—H or C—NH₂, then Y₄ is not C—H
(xi) when Z is S and Y₁ is N, then Y₂ is not C-halogen, C-alkyl, C-cycloalkyl, C-aryl, C-heteroaryl, C—CH₂NH₂, C—COOalkyl, or C—NHC(O)alkyl; (xii) when Z is NR₂, then Y₁, Y₂ and Y₄ are CR.

In certain embodiments, the compound has the structure:

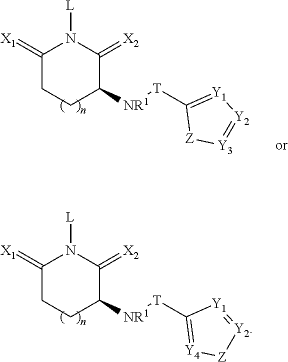

In other embodiments, the compound has the structure:

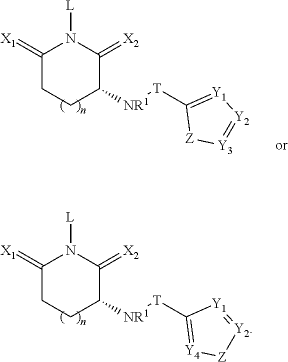

In some embodiments, T is C═O. In other embodiments, T is SO₂.

In some embodiments, each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NR″C(O)R″, —NR″C(O)CH(OH)R″, —NR″C(O)OR″, —NR″SO₂R″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, or —S(O)₂R″, —S(O)₂OR″, —S(O)₂NH₂, —S(O)₂NHR″, or —S(O)₂NR″₂.

In some embodiments, Z is S or NR². In some embodiments, Z is NR². In other embodiments, Z is S.

In some embodiments, L is hydrogen, alkyl, alkenyl, haloalkyl, haloalkenyl, —C(O)R″, —CH₂C(O)OR″, —C(O)OR″, —C(O)NH₂, —C(O)NHR″, —C(O)NR″₂, —OR″, —NR″₂, or —S(O)₂R″. In some embodiments, L is hydrogen, alkyl, alkenyl, —CH₂C(O)OR″, —OR″, —NR″₂, or —S(O)₂R″; optionally wherein L is hydrogen, alkyl, or alkenyl. In some embodiments, L is hydrogen, alkyl, —CH₂C(O)OR″ or —OR″.

In some embodiments, L is hydrogen, alkyl, alkenyl, aryl, heteroaryl, benzyl, haloalkyl, haloalkenyl, —OR″, —NR″₂, or —S(O)₂R″. In other embodiments, L is hydrogen, alkyl, alkenyl, aryl, heteroaryl, benzyl, haloalkyl, haloalkenyl, —C(O)R″, —C(O)OR″, —C(O)NH₂, —C(O)NHR″, or —C(O)NR″₂. In some embodiments, L is hydrogen, alkyl, alkenyl, aryl, heteroaryl, benzyl, haloalkyl, haloalkenyl, —OR″, —NR″₂, or —S(O)₂R″. In some embodiments, L is hydrogen, alkyl, alkenyl, aryl, heteroaryl, benzyl, haloalkyl, or haloalkenyl. In other embodiments, L is —OR″, —NR″₂, or —S(O)₂R″ In some embodiments, L is hydrogen, alkyl, alkenyl, aryl, heteroaryl, or benzyl. In some embodiments, L is hydrogen, alkyl, alkenyl, or aryl. In some embodiments, L is hydrogen, alkyl, or alkenyl. In some embodiments, L is hydrogen or alkyl. In some embodiments, L is hydrogen.

In some embodiments, R² is hydrogen, alkyl, cycloalkyl, aryl, heteroaryl, benzyl, haloalkyl, haloalkenyl, —NR″₂, —NR″C(O)R″, —N[C(O)R″]₂, —NR″C(O)OR″, —C(O)R″, —C(O)OR″, —OR″, —OC(O)R″, —OC(O)OR″, —OC(O)NH₂, —OC(O)NHR″, or —OC(O)NR″₂. In some such embodiments, R² is alkyl, benzyl, or —N[C(O)R″]₂.

In some embodiments, the compound is of Formula (Ia) wherein one of Y₁, Y₂ and Y₃ is N, and the remaining two of Y₁, Y₂ and Y₃ are each CR. In some such embodiments, Y₁ is N, and Y₂ and Y₃ are CR. In other such embodiments, Y₂ is N; Y₁ and Y₃ are CR; and Z is S. In other such embodiments, Y₃ is N; Y₁ and Y₂ are CR; and Z is S.

In some embodiments, the compound is of Formula (Ia), wherein one of Y₁, Y₂ and Y₃ is CR; the remaining two of Y₁, 1¹₂ and Y₃ are each N; and Z is S. In some such embodiments, Y₁ is CR; Y₂ and Y₃ are N. In other such embodiments, Y₂ is CR; Y₁ and Y₃ are N. In other such embodiments, Y₃ is CR, and Y₁ and Y₂ are N.

In some embodiments, the compound is of Formula (Ia) and Y₁, Y₂ and Y₃ are each CR. In some such embodiments, Y₁ is —C—NHC(O)R″, Y₂ is CH, and Y₃ is CH or CCl. In some such embodiments, L is hydrogen; Z is 5; R′ is H; T is C═O; Y₁ is —C—NHC(O)R″; Y₂ is CH; and Y₃ is CH.

In some embodiments, the compound is of Formula (Ib), wherein one of Y₁, Y₂ and Y₄ is N and the remaining two of Y₁, 1¹₂ and Y₄ are each CR, and wherein Z is S. In some such embodiments, Y₁ is N, and Y₂ and Y₄ are CR. In other such embodiments, Y₂ is N, and Y₁ and Y₄ are CR. In other such embodiments, Y₄ is N, and Y₁ and Y₂ are CR.

In some embodiments, the compound is of Formula (Ib), wherein one of Y₁, Y₂ and Y₄ is CR and the remaining two of Y₁, Y₂ and Y₄ are each N, and wherein Z is S. In some such embodiments, Y₁ is CR, and Y₂ and Y₄ are N. In other such embodiments, Y₂ is CR, and Y₁ and Y₄ are N. In other such embodiments, Y₄ is CR, and Y₁ and Y₂ are N.

In some embodiments, the compound is of Formula (Ib) and Y₁, Y₂ and Y₄ are each CR. In some such embodiments, each R is independently hydrogen, halogen, alkyl, cycloalkyl, haloalkyl, heteroaryl, —OR″, —N[C(O)R″]₂, —NR″C(O)R″, —NHC(O)OR″, —NHR″, —NH₂, or —NHSO₂R″CN. In some such embodiments, each R″ is independently alkyl, cycloalkyl, aryl or benzyl. In some such embodiments:
L is hydrogen;
Z is S;
R¹ is H;
T is C═O;
Y₁ is CH, C—OR″, CCl, C—CN, or C—NHC(O)R″;
Y₂ is CH, CCl, C-alkyl, C-cycloalkyl, or C-haloalkyl; and
Y₄ is CH, C—OR″, C—NHC(O)R″, C—NHC(O)OR″, C—NHR″, C—NH₂, or C—NHSO₂R″;
wherein, when Y₁ is CCl, then Y₂ is CH, C-alkyl, C-cycloalkyl, or C-haloalkyl.

In some such embodiments each R″ is independently alkyl, cycloalkyl, aryl or benzyl. In some such embodiments, Y₁ is CH; Y₂ is CH or CCl; and Y₄ is C—OR″ or C—NH₂, optionally C—OMe or C—NH₂.

In some embodiments of the compound of Formula (Ia) or Formula (Ib), each R is independently hydrogen, halogen, alkyl, cycloalkyl, haloalkyl, heteroaryl, —NR″C(O)R″, NR″C(O)OR″, —NR″C(O)CH(OH)R″, —NHR″, —NH₂, —OR″, —CN, —C(O)NR″₂, or —NR″SO₂R″. In some such embodiments, each R is independently hydrogen, halogen, alkyl, cycloalkyl, haloalkyl, —OR″, —CN, —NHC(O)R″, —NHC(O)OR″, —NHR″, —NH₂ or —NHSO₂R″. In some embodiments, each R″ is independently alkyl, cycloalkyl, aryl or benzyl.

In some embodiments of the compound of Formula (Ia) or (Ib), X₁ and X₂ are O. In other embodiments, X₁ is O and X₂ is S. In other embodiments, X₁ is S and X₂ is 0. In other embodiments, X₁ and X₂ are S.

In some embodiments of the compound of Formula (Ia) or (Ib), n is 0. In other embodiments of the compound of Formula (Ia) or (Ib), n is 1 or 2. In some embodiments, n is 1. In other embodiments, n is 2.

In some embodiments, the compound is of Formula (Ia), wherein:
X₁ and X₂ are O
T is C═O
n is 1
R¹ is hydrogen
L is hydrogen, and
Z is S,
   wherein,
when Y₁ and Y₃ are CR, then
   (a) Y₂ is CH, Y₃ is CH, and Y₁ is C—NHC(O)R″;
   (b) Y₁ is CH, Y₃ is CH, and Y₂ is C—OH or C—CH₂NHC(O)OR″; or
   (c) Y₁ is CH, Y₂ is CH, and Y₃ is C—CH₂NHC(O)OR″; and
      when Y₂ is N, then Y₃ is C—NHC(O)OR″ or C—NH₂.

In some embodiments, the compound is of Formula (Ib), wherein:
X₁ and X₂ are O
T is C═O
n is 1
R¹ is hydrogen
L is hydrogen
Z is S, and
Y₂ and Y₄ are CR,
   wherein:
when Y₁ is N, then Y₂ is CH, C—NH₂ or C—CH₂NHC(O)OR″ and Y₄ is CH or C—OR″, wherein at least one of Y₂ and Y₄ is not CH; and
when Y₁, Y₂ and Y₄ are each CR, then
   Y₁ is CH, C-alkyl, C—Cl or C—OR″
   Y₂ is CH, C-halogen, C-cycloalkyl, C-haloalkyl, C-aryl, C—CONH(R″), or C—CN
   Y₄ is CH, C—NH₂, C—NHR″, C—NR″C(O)R″, C—NR″SO₂R″, or C—OR″;
   at least one of Y₁, Y₂ and Y₄ is CH or C—Cl; and
   (a) when Y₁ and Y₂ are CH, then Y₄ is C—NHCOR″, C—NHSO₂R″, C—OR″, or C—NH₂,
   (b) when Y₄ is C—NHCOR″ and Y₁ is C—Cl, then Y₂ is C-cycloalkyl, and
   (c) when Y₁ and Y₂ are CH and Y₄ is C—OMe, then the compound is compound 20 or compound 21.

In accordance with a second aspect of the invention, there is provided a compound of Formula (IIa) or (IIb):

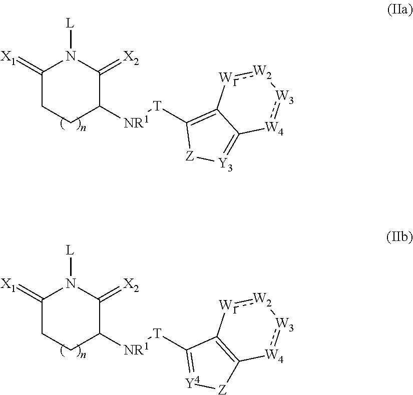

or a pharmaceutically acceptable salt or tautomer thereof,
wherein
each of X₁ and X₂ is independently O or S;
Z is O, S or NR²;
T is C═O or SO₂;
Y₃ is N or CR;
Y₄ is N or CR;
[symbol] indicates a single or double bond, wherein
   when each [symbol] is a double bond, each of W₁, W₂, W₃ and W₄ is independently N or CR′, wherein at least one of W₁, W₂, W₃ and W₄ is N, and
   when each [symbol] is a single bond, W₁, W₂, W₃ and W₄ are each CR′₂ and Y₄ is CR;
n is 0, 1 or 2;
L is hydrogen, alkyl, alkenyl, aryl, heteroaryl, benzyl, haloalkyl, haloalkenyl, —C(O)R″, —C(O)OR″, —C(O)NH₂, —C(O)NHR″, —C(O)NR″₂, —OR″, —NR″₂, or —S(O)₂R″;
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NR″C(O)R″, —NR″C(O)CH₂R″, —NR″C(O)CH(OH)R″, —NR″C(O)OR″, —NR″SO₂R″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, or —S(O)₂R″, —S(O)₂OR″, —S(O)₂NH₂, —S(O)₂NHR″, or —S(O)₂NR″₂;
each R′ is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NR″C(O)R″, —NR″C(O)CH(OH)R″, —NR″C(O)OR″, —NR″SO₂R″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, or —S(O)₂R″, —S(O)₂OR″, —S(O)₂NH₂, —S(O)₂NHR″, or —S(O)₂NR″₂;
each R″ is independently hydrogen, alkyl, cycloalkyl, heterocycloalkyl, alkenyl, cycloalkenyl, aryl, heteroaryl, or benzyl;
R² is hydrogen, alkyl, cycloalkyl, alkenyl, cycloalkenyl, alkynyl, aryl, heteroaryl, benzyl, haloalkyl, haloalkenyl, —NH₂, —NHR″, —NR″₂, —NR″C(O)R″, —N[C(O)R″]₂, —NR″C(O)OR″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, or —S(O)₂R″, —S(O)₂OR″, —S(O)₂NH₂, —S(O)₂NHR″, or —S(O)₂NR″₂; and
R¹ is hydrogen, alkyl, cycloalkyl, alkenyl, cycloalkenyl, aryl, heteroaryl, or benzyl;
wherein when each [symbol] is a double bond, Z is NR², R² is hydrogen, and each R′ is hydrogen, then W₄ is CR′.

In some embodiments, the compound is of Formula (IIa).

In some embodiments, the compound is of Formula (IIb).

In some embodiments, the compound of Formula (IIa) or (IIb) has the structure:

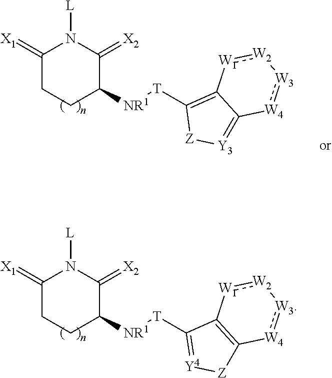

In other embodiments, the compound of Formula (IIa) or (IIb) has the structure:

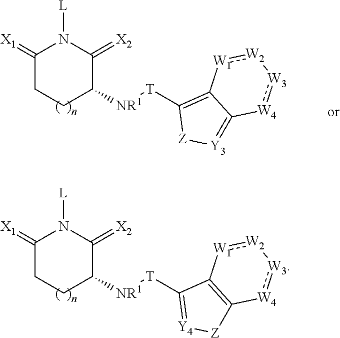

In some embodiments, when each [symbol] is a double bond, Z is NR², R² is hydrogen, and Y₃ and Y₄ are CR, one of W₁, W₂, W₃ and W₄ is N, and the remaining three of W₁, W₂, W₃ and W₄ are each CR′, then at least one R′ is not hydrogen.

In some embodiments of the compound of Formula (IIa) or (IIb), Z is O. In other embodiments, Z is S. In other embodiments, Z is NR².

In some embodiments of the compound of Formula (IIa) or (IIb), T is C═O. In other embodiments, T is SO₂.

In some embodiments, L is hydrogen, alkyl, alkenyl, aryl, heteroaryl, benzyl, haloalkyl, haloalkenyl, —C(O)R″, —C(O)OR″, —C(O)NH₂, —C(O)NHR″, —C(O)NR″₂, —NR″₂, or —S(O)₂R″; optionally wherein L is hydrogen, alkyl, alkenyl, aryl, heteroaryl, benzyl, —NR″₂, or —S(O)₂R″

In some embodiments of the compound of Formula (IIa) or (IIb), L is hydrogen, alkyl, alkenyl, aryl, heteroaryl, benzyl, haloalkyl, haloalkenyl, —OR″, —NR″₂, or —S(O)₂R″. In other embodiments of the compound of Formula (IIa) or (IIb), L is hydrogen, alkyl, alkenyl, aryl, heteroaryl, benzyl, haloalkyl, haloalkenyl, —C(O)R″, —C(O)OR″, —C(O)NH₂, —C(O)NHR″, or —C(O)NR″₂. In some embodiments of the compound of Formula (IIa) or (IIb), L is hydrogen, alkyl, alkenyl, aryl, heteroaryl, benzyl, haloalkyl, haloalkenyl, —OR″, —NR″₂, or —S(O)₂R″. In some embodiments of the compound of Formula (IIa) or (IIb), L is hydrogen, alkyl, alkenyl, aryl, heteroaryl, benzyl, haloalkyl, or haloalkenyl. In other embodiments of the compound of Formula (IIa) or (IIb), L is —OR″, —NR″₂, or —S(O)₂R″ In some embodiments of the compound of Formula (IIa) or (IIb), L is hydrogen, alkyl, alkenyl, aryl, heteroaryl, or benzyl. In some embodiments of the compound of Formula (IIa) or (IIb), L is hydrogen, alkyl, alkenyl, or aryl. In some embodiments of the compound of Formula (IIa) or (IIb), L is hydrogen, alkyl, or alkenyl. In some embodiments of the compound of Formula (IIa) or (IIb), L is hydrogen or alkyl. In some embodiments of the compound of Formula (IIa) or (IIb), L is hydrogen.

In some embodiments of the compound of Formula (IIa), Y₃ is N. In other embodiments, Y₃ is CR.

In some embodiments of the compound of Formula (IIb), Y₄ is N. In other embodiments, Y₄ is CR.

In some embodiments, [symbol] indicates a single bond between W₁ and W₂, and W₁ and W₂ are each CR′₂. In other embodiments, [symbol] indicates a double bond between W₁ and W₂, and W₁ and W₂ are each independently N or CR′. In either of the two aforementioned embodiments, [symbol] indicates a single bond between W₃ and W₄, and W₃ and W₄ are each CR′₂. Alternatively, [symbol] indicates a double bond between W₃ and W₄, and W₃ and W₄ are each independently N or CR′.

In some embodiments of the compound of Formula (IIa) or (IIb), each [symbol] is a double bond or each [symbol] is a single bond.

In some embodiments, each [symbol] is a double bond. In some such embodiments, one of W₁, W₂, W₃ and W₄ is N, and the remaining three of W₁, W₂, W₃ and W₄ are each CR′. In some such embodiments, one of W₁, W₂ and W₃ is N, and W₄ is CR′. In some such embodiments W₁ is N, and W₂, W₃ and W₄ are each CR′. In other such embodiments, W₂ is N, and W₁, W₃ and W₄ are each CR′. In other such embodiments, W₃ is N, and W₁, W₂ and W₄ are each CR′. In other such embodiments, W₄ is N, and W₂ and W₃ are each CR′. In other embodiments wherein each [symbol] is a double bond, two of W₁, W₂, W₃ and W₄ is N, and the remaining two of W₁, W₂, W₃ and W₄ are each CR′. In some such embodiments W₁ and W₂ are each N, and W₃ and W₄ are each CR′. In other such embodiments, W₁ and W₃ are each N, and W₂ and W₄ are each CR′. In other such embodiments, W₁ and W₄ are each N, and W₂ and W₃ are each CR′. In other such embodiments, W₂ and W₃ are each N, and W₁ and W₄ are each CR′. In other such embodiments, W₂ and W₄ are each N, and W₁ and W₃ are each CR′. In other such embodiments, W₃ and W₄ are each N, and W₁ and W₂ are each CR′.

In other embodiments wherein each [symbol] is a double bond, one of W₁, W₂, W₃ and W₄ is CR′, and the remaining three of W₁, W₂, W₃ and W₄ are each N. In some such embodiments W₁ is CR′, and W₂, W₃ and W₄ are each N. In other such embodiments, W₂ is CR′, and W₁, W₃ and W₄ are each N. In other such embodiments, W₃ is CR′, and W₁, W₂ and W₄ are each N. In other such embodiments, W₄ is CR′, and W₁, W₂ and W₃ are each N.

In some embodiments, at least one R′ is not hydrogen.

In some embodiments of the compound of Formula (IIa) or (IIb), each [symbol] is a single bond.

In some embodiments of the compound of Formula (IIa) or (IIb), each R is independently hydrogen, halogen or —NR″C(O)R″.

In some embodiments of the compound of Formula (IIa) or (IIb), each R′ is hydrogen.

In some embodiments of the compound of Formula (IIa) or (IIb), X₁ and X₂ are O. In other embodiments,

X₁ is O and X₂ is S. In other embodiments, X₁ is S and X₂ is 0. In other embodiments, X₁ and X₂ are S.

In some embodiments of the compound of Formula (IIa) or (IIb), n is 0. In other embodiments of the compound of Formula (IIa) or (IIb), n is 1 or 2. In some embodiments, n is 1. In other embodiments, n is 2.

In some embodiments, the compound is of Formula (IIa), wherein:
X₁ and X₂ are O
T is C═O
n is 1
R¹ is hydrogen
L is hydrogen
Z is S
each [symbol] is a double bond
W₁ is N
W₂, W₃ and W₄ are each independently CH or C—Cl; and
Y₃ is CH or C-halogen.

In some embodiments, the compound is of Formula (IIb), wherein:
X₁ and X₂ are O
T is C═O
n is 1
R¹ is hydrogen
L is hydrogen
Z is NH
each [symbol] is a double bond
W₄ is N
W₃ is CH
one of W₁ and W₂ is C—Cl, and the other of W₁ and W₂ is CH; and
Y₄ is CH.

In accordance with a third aspect, the present invention provides a compound of Formula (Ia), Formula (Ib), Formula (IIa) or Formula (IIb) selected from:

| Com- | |
|---|---|
| pound | |
| ID | Structure |
| 3 | 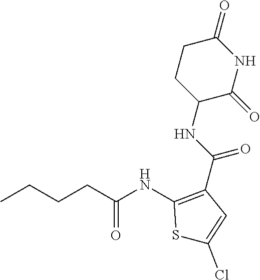 [SMILES: CCCCC(=O)Nc1sc(Cl)cc1C(=O)NC1CCC(=O)NC1=O] |
| 4 | 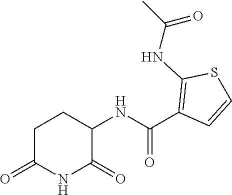 [SMILES: CC(=O)Nc1sccc1C(=O)NC1CCC(=O)NC1=O] |
| 6 | 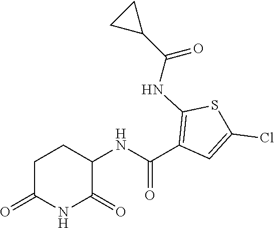 [SMILES: O=C1CCC(NC(=O)c2cc(Cl)sc2NC(=O)C2CC2)C(=O)N1] |
| 7 | 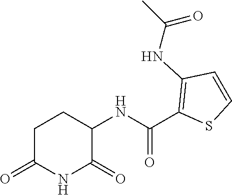 [SMILES: CC(=O)Nc1ccsc1C(=O)NC1CCC(=O)NC1=O] |
| 8 | 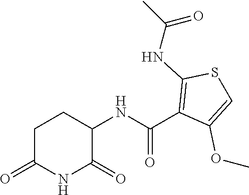 [SMILES: COc1csc(NC(C)=O)c1C(=O)NC1CCC(=O)NC1=O] |
| 9 | 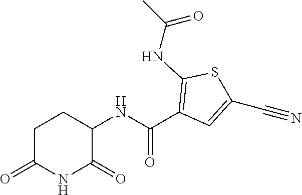 [SMILES: CC(=O)Nc1sc(C#N)cc1C(=O)NC1CCC(=O)NC1=O] |
| 12 | 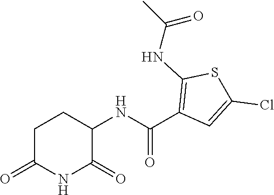 [SMILES: CC(=O)Nc1sc(Cl)cc1C(=O)NC1CCC(=O)NC1=O] |
| 13 | 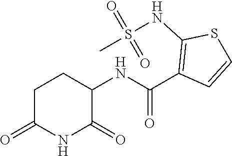 [SMILES: CS(=O)(=O)Nc1sccc1C(=O)NC1CCC(=O)NC1=O] |
| 14 | 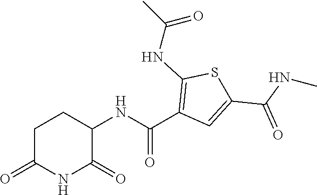 [SMILES: CNC(=O)c1cc(C(=O)NC2CCC(=O)NC2=O)c(NC(C)=O)s1] |
| 15 | 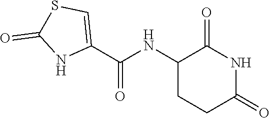 [SMILES: O=C1CCC(NC(=O)c2csc(=O)[nH]2)C(=O)N1] |
| 16 | 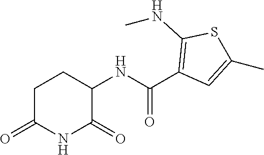 [SMILES: CNc1sc(C)cc1C(=O)NC1CCC(=O)NC1=O] |
| 19 | 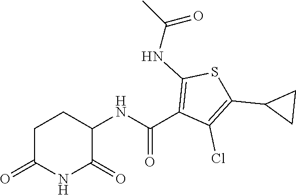 [SMILES: CC(=O)Nc1sc(C2CC2)c(Cl)c1C(=O)NC1CCC(=O)NC1=O] |
| 20 | 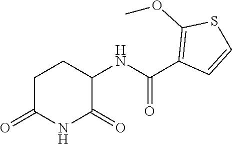 [SMILES: COc1sccc1C(=O)NC1CCC(=O)NC1=O] |
| 21 | 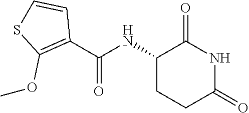 [SMILES: COc1sccc1C(=O)N[C@H]1CCC(=O)NC1=O] |
| 28 | 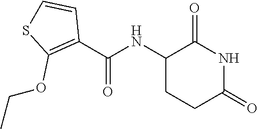 [SMILES: CCOc1sccc1C(=O)NC1CCC(=O)NC1=O] |
| 29 | 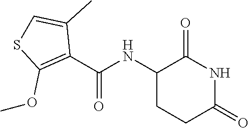 [SMILES: COc1scc(C)c1C(=O)NC1CCC(=O)NC1=O] |
| 30 | 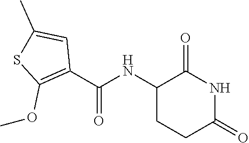 [SMILES: COc1sc(C)cc1C(=O)NC1CCC(=O)NC1=O] |
| 32 | 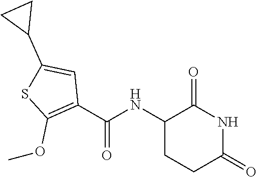 [SMILES: COc1sc(C2CC2)cc1C(=O)NC1CCC(=O)NC1=O] |
| 33 | 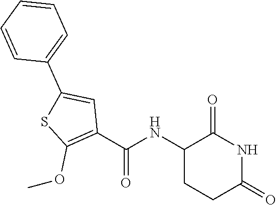 [SMILES: COc1sc(-c2ccccc2)cc1C(=O)NC1CCC(=O)NC1=O] |
| 34 | 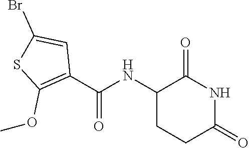 [SMILES: COc1sc(Br)cc1C(=O)NC1CCC(=O)NC1=O] |
| 35 | 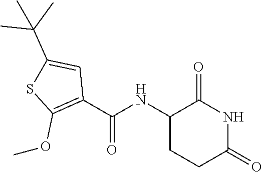 [SMILES: COc1sc(C(C)(C)C)cc1C(=O)NC1CCC(=O)NC1=O] |
| 36 | 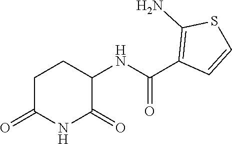 [SMILES: Nc1sccc1C(=O)NC1CCC(=O)NC1=O] |
| 37 | 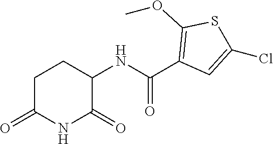 [SMILES: COc1sc(Cl)cc1C(=O)NC1CCC(=O)NC1=O] |
| 40 | 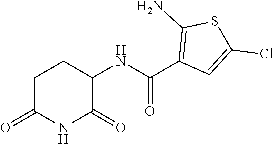 [SMILES: Nc1sc(Cl)cc1C(=O)NC1CCC(=O)NC1=O] |
| 42 | 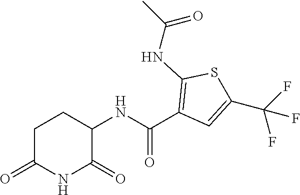 [SMILES: CC(=O)Nc1sc(C(F)(F)F)cc1C(=O)NC1CCC(=O)NC1=O] |
| 44 | 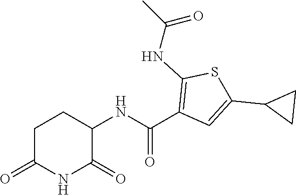 [SMILES: CC(=O)Nc1sc(C2CC2)cc1C(=O)NC1CCC(=O)NC1=O] |
| 46 | 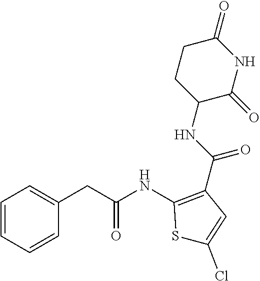 [SMILES: O=C1CCC(NC(=O)c2cc(Cl)sc2NC(=O)Cc2ccccc2)C(=O)N1] |
| 49 | 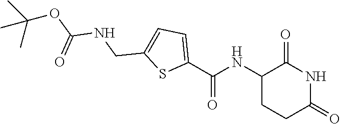 [SMILES: CC(C)(C)OC(=O)NCc1ccc(C(=O)NC2CCC(=O)NC2=O)s1] |
| 51 | 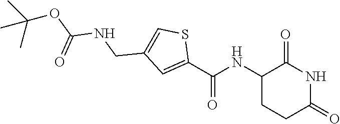 [SMILES: CC(C)(C)OC(=O)NCc1csc(C(=O)NC2CCC(=O)NC2=O)c1] |
| 53 | 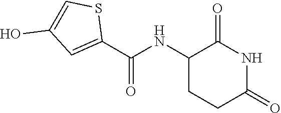 [SMILES: O=C1CCC(NC(=O)c2cc(O)cs2)C(=O)N1] |
| 54 | 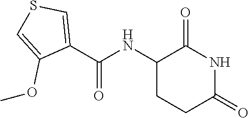 [SMILES: COc1cscc1C(=O)NC1CCC(=O)NC1=O] |
| 57 | 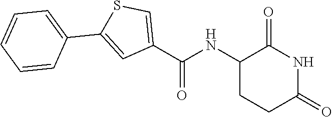 [SMILES: O=C1CCC(NC(=O)c2csc(-c3ccccc3)c2)C(=O)N1] |
| 71 | 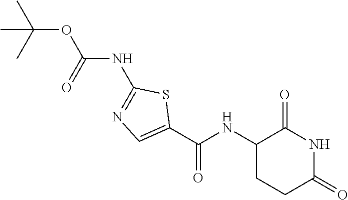 [SMILES: CC(C)(C)OC(=O)Nc1ncc(C(=O)NC2CCC(=O)NC2=O)s1] |
| 77 | 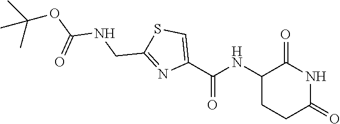 [SMILES: CC(C)(C)OC(=O)NCc1nc(C(=O)NC2CCC(=O)NC2=O)cs1] |
| 79 | 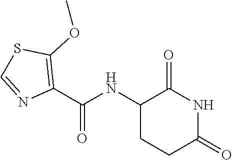 [SMILES: COc1scnc1C(=O)NC1CCC(=O)NC1=O] |
| 80 | 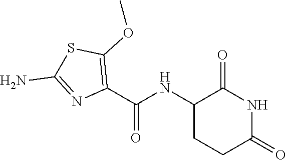 [SMILES: COc1sc(N)nc1C(=O)NC1CCC(=O)NC1=O] |
| 96 | 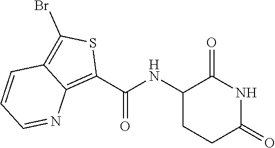 [SMILES: O=C1CCC(NC(=O)c2sc(Br)c3cccnc23)C(=O)N1] |
| 97 | 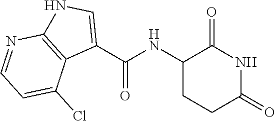 [SMILES: O=C1CCC(NC(=O)c2c[nH]c3nccc(Cl)c23)C(=O)N1] |
| 99 | 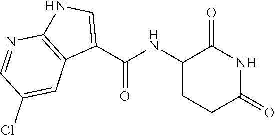 [SMILES: O=C1CCC(NC(=O)c2c[nH]c3ncc(Cl)cc23)C(=O)N1] |
| 106 | 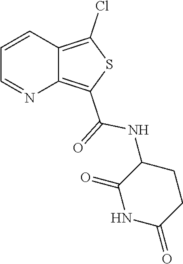 [SMILES: O=C1CCC(NC(=O)c2sc(Cl)c3cccnc23)C(=O)N1] |
| 107 | 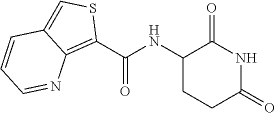 [SMILES: O=C1CCC(NC(=O)c2scc3cccnc23)C(=O)N1] |
| 112 | 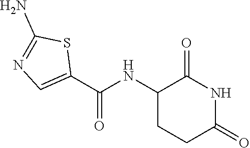 [SMILES: Nc1ncc(C(=O)NC2CCC(=O)NC2=O)s1] |

In accordance with a fourth aspect of the invention, there is provided a pharmaceutical composition comprising a compound according to any of the above aspects of the present invention.

The invention also provides a compound according to any of the above aspects of the present invention for use as a cereblon binder.

The invention also provides a compound or composition according to any of the above aspects of the present invention, or a compound selected from

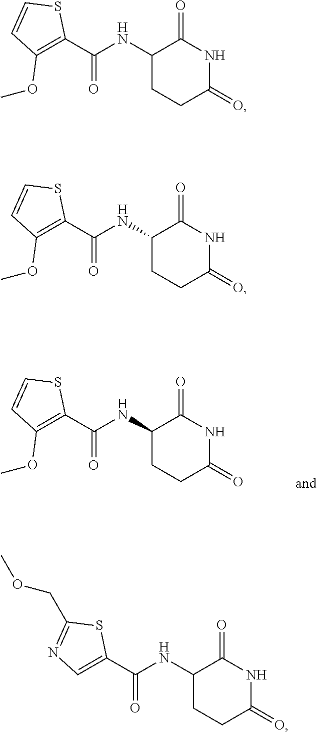 [SMILES: COc1ccsc1C(=O)NC1CCC(=O)NC1=O ; COc1ccsc1C(=O)N[C@H]1CCC(=O)NC1=O ; COc1ccsc1C(=O)N[C@@H]1CCC(=O)NC1=O ; COCc1ncc(C(=O)NC2CCC(=O)NC2=O)s1]

for use in medicine.

The invention also provides a compound or composition according to any of the above aspects of the present invention, or a compound selected from

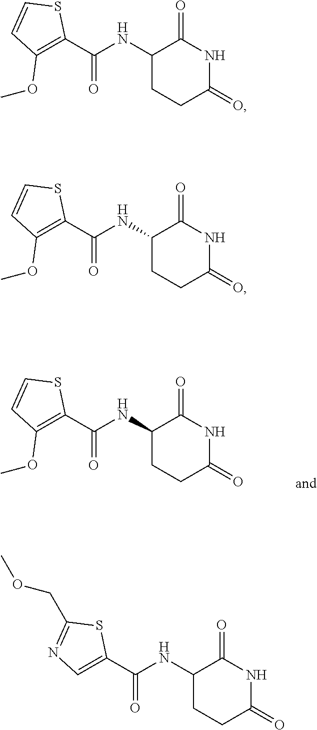 [SMILES: COc1ccsc1C(=O)NC1CCC(=O)NC1=O ; COc1ccsc1C(=O)N[C@H]1CCC(=O)NC1=O ; COc1ccsc1C(=O)N[C@@H]1CCC(=O)NC1=O ; COCc1ncc(C(=O)NC2CCC(=O)NC2=O)s1]

for use in immune-oncology.

The invention also provides a compound or composition according to any of the above aspects of the present invention, or a compound selected from

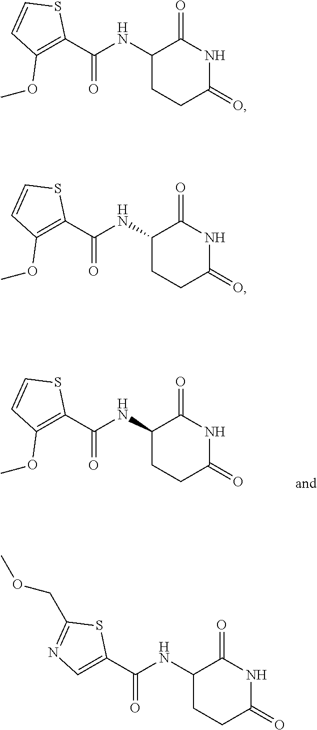 [SMILES: COc1ccsc1C(=O)NC1CCC(=O)NC1=O ; COc1ccsc1C(=O)N[C@H]1CCC(=O)NC1=O ; COc1ccsc1C(=O)N[C@@H]1CCC(=O)NC1=O ; COCc1ncc(C(=O)NC2CCC(=O)NC2=O)s1]

for use in the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders.

In some embodiments, the compound is a compound of any one of the first to third aspects of the present invention, or a compound selected from

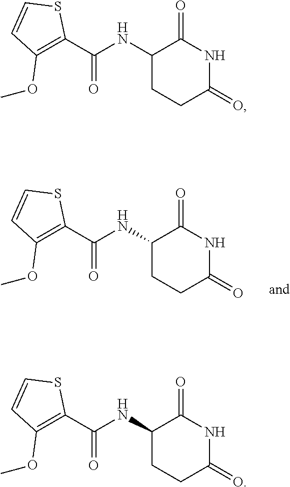 [SMILES: COc1ccsc1C(=O)NC1CCC(=O)NC1=O ; COc1ccsc1C(=O)N[C@H]1CCC(=O)NC1=O ; COc1ccsc1C(=O)N[C@@H]1CCC(=O)NC1=O]

In some embodiments, the compound is a compound of any one of the first to third aspects of the present invention.

The present invention also provides a method for the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders; wherein the method comprises administering to a patient in need thereof an effective amount of a compound or composition according to any of the above aspects of the present invention, or a compound selected from

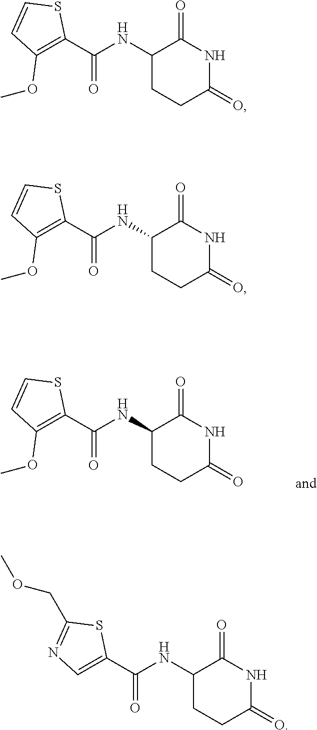 [SMILES: COc1ccsc1C(=O)NC1CCC(=O)NC1=O ; COc1ccsc1C(=O)N[C@H]1CCC(=O)NC1=O ; COc1ccsc1C(=O)N[C@@H]1CCC(=O)NC1=O ; COCc1ncc(C(=O)NC2CCC(=O)NC2=O)s1]

In some embodiments of the method, the method further comprises administering at least one additional active agent to the patient. In some embodiments, the at least one additional active agent is an anti-cancer agent or an agent for the treatment of an autoimmune disease. In some embodiments, the at least one additional active agent is a peptide, an antibody, a corticosteroid, or a combination thereof. In some embodiments, the at least one additional active agent is at least one of bortezomib, dexamethasone, and rituximab.

In some embodiments, an effective amount of a compound or composition according to any of the above aspects of the present invention is administered to the patient.

The present invention also provides a combined preparation of a compound of any one of the first to third aspects of the present invention, or a compound selected from

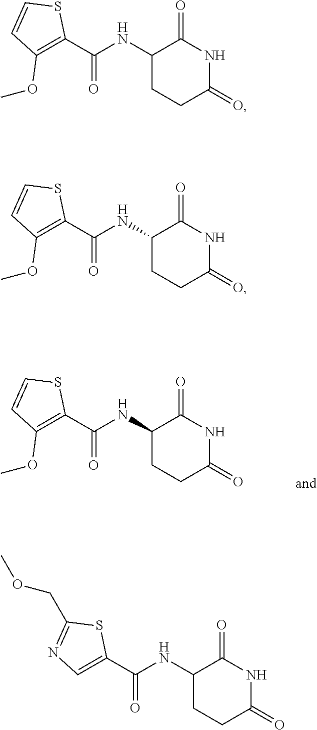 [SMILES: COc1ccsc1C(=O)NC1CCC(=O)NC1=O ; COc1ccsc1C(=O)N[C@H]1CCC(=O)NC1=O ; COc1ccsc1C(=O)N[C@@H]1CCC(=O)NC1=O ; COCc1ncc(C(=O)NC2CCC(=O)NC2=O)s1]

and at least one additional active agent, for simultaneous, separate or sequential use in therapy.

The present invention also provides a combined preparation of a compound of any one of the first to third aspects of the present invention, and at least one additional active agent, for simultaneous, separate or sequential use in therapy.

In some embodiments of the combined preparation, the at least one additional active agent is an anti-cancer agent or an agent for the treatment of an autoimmune disease. In some embodiments, the at least one additional active agent is a small molecule, a peptide, an antibody, a corticosteroid, or a combination thereof. In some embodiments, the at least one additional active agent is at least one of bortezomib, dexamethasone, and rituximab. In some embodiments, the therapy is the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders.

As used herein the term “alkyl” is intended to include both unsubstituted alkyl groups, and alkyl groups which are substituted by one or more additional groups—for example —OH, —OR″, —NH₂, —NHR″, —NR″₂, —SO₂R″, —C(O)R″, —CN, or —NO₂. In some embodiments, the alkyl group is an unsubstituted alkyl group. In some embodiments, the alkyl group is a C₁-C₁₂ alkyl, a C₁-C₁₀ alkyl, a C₁-C₈ alkyl, a C₁-C₆ alkyl, or a C₁-C₄ alkyl group.

As used herein the term “alkenyl” is intended to include both unsubstituted alkenyl groups, and alkenyl groups which are substituted by one or more additional groups—for example —OH, —OR″, —NH₂, —NHR″, —NR″₂, —SO₂R″, —C(O)R″, —CN, or —NO₂. In some embodiments, the alkenyl group is an unsubstituted alkenyl group. In some embodiments, the alkenyl group is a C₂-C₁₂ alkenyl, a C₂-C₁₀ alkenyl, a C₂-C₈ alkenyl, a C₂-C₆ alkenyl, or a C₂-C₄ alkenyl group.

As used herein the term “alkynyl” is intended to include both unsubstituted alkynyl groups, and alkynyl groups which are substituted by one or more additional groups—for example —OH, —OR″, halogen, —NH₂, —NHR″, —NR″₂, —SO₂R″, —C(O)R″, —CN, or —NO₂. In some embodiments, the alkynyl group is an unsubstituted alkynyl group. In some embodiments, the alkynyl group is a C₂-C₁₂ alkynyl, a C₂-C₁₀ alkynyl, a C₂-C₈ alkynyl, a C₂-C₆ alkynyl, or a C₂-C₄ alkynyl group.

As used herein the term “aryl” is intended to include both unsubstituted aryl groups, and aryl groups which are substituted by one or more additional groups—for example —OH, —OR″, halogen, —NH₂, —NHR″, —NR″₂, —SO₂R″, —C(O)R″, —CN, or —NO₂. In some embodiments, the aryl group is an unsubstituted aryl group. In some embodiments, the aryl group is a C₆-C₁₀ aryl, a C₆-C₈ aryl, or a C₆ aryl.

As used herein the term “heteroaryl” is intended to include both unsubstituted heteroaryl groups, and heteroaryl groups which are substituted by one or more additional groups—for example —OH, —OR″, halogen, —NH₂, —NHR″, —NR″₂, —SO₂R″, —C(O)R″, —CN, or —NO₂. In some embodiments, the heteroaryl group is an unsubstituted heteroaryl group. In some embodiments, the heteroaryl group is a C₆-C₁₀ heteroaryl, a C₆-C₉ heteroaryl, a C₆-C₈ heteroaryl, or a C₆ heteroaryl.

As used herein the term “benzyl” is intended to include both unsubstituted benzyl groups, and benzyl groups which are substituted by one or more additional groups—for example —OH, —OR″, halogen, —NH₂, —NHR″, —NR″₂, —SO₂R″, —C(O)R″, —CN, or —NO₂. In some embodiments, the benzyl group is an unsubstituted benzyl group.

In some embodiments, all alkyl, cycloalkyl, heterocycolalkyl, alkenyl, alkynyl, aryl, heteroaryl, benzyl groups are unsubstituted.

### BRIEF DESCRIPTION OF THE FIGURES

FIG. 1 is an assay showing the effect of various compounds of the invention and various reference compounds CK1α degradation in the Kelly cell line.

FIG. 2 is an assay showing the effect of various compounds of the invention and various reference compounds on IKZF1 degradation in the H929 cell line.

FIG. 3 is an assay showing the effect of various compounds of the invention and various reference compounds on IKZF3 degradation in the H929 cell line.

### DETAILED DESCRIPTION OF THE INVENTION

As discussed above, the present invention provides compounds of Formulas (la), (Ib), (IIa) and (IIb):

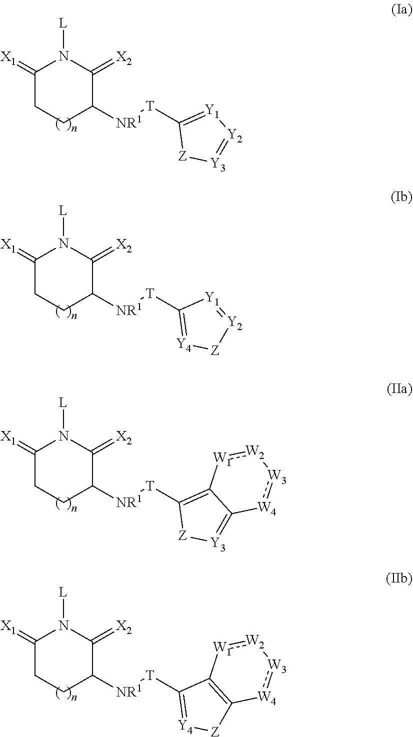

wherein L, X₁, X₂, Y₁, Y₂, Y₃, Y₄, W₁, W₂, W₃, W₄, R₁ and Z are as defined above.

Binding of the above compounds to cereblon may alter the specificity of the CRL4^{CRBN} complexes, and induce association of novel substrate proteins, followed by their ubiquitination and degradation. Examples of such proteins include, but are not limited to, IKZF1 and IKZF3.

The above compounds may modulate cereblon in a unique way allowing CRL4^{CRBN} ubiquitin ligase complex to recognise different substrates to those which it would otherwise recognise, and target them for degradation. Consequently, the compounds of the present invention are expected to broaden/modify CRBN's antiproliferative activity, thus extending the range of cancer types sensitive to treatment with CMAs.

The compounds of the present invention are advantageous in terms of their synthetic feasibility. The synthesis of the compounds can be summarized in the following general procedure (carried out under Synthetic Conditions A or Synthetic Conditions B, as set out below:

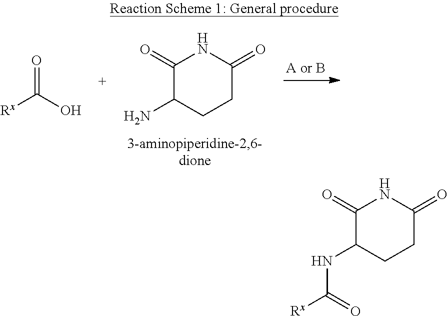

Synthetic Conditions A

An appropriate acid (R′COOH in the above reaction scheme) (1.1 eq), DMAP (0.04 eq), and EDC (1.2 eq) were added to a solution of 3-aminopiperidine-2,6-dione (1 eq) and N-hydroxybenzotriazole (1.2 eq) in DMF (0.5 M). The reaction mixture was stirred overnight at room temperature (20-25° C.). Water (2×DMF volume) was added and the obtained solution was extracted with dichloromethane (3×DMF volume). The combined organic layers were washed with water, dried over Na₂SO₄, and concentrated under reduced pressure. The crude product was purified by preparative HPLC or by column chromatography.

Synthetic Conditions B

An appropriate acid R^{x}COOH in the above reaction scheme) (1 eq) and EDC (1.2 eq) were added to a solution of 3-aminopiperidine-2,6-dione (hydrochloride salt, 1.1 eq), triethylamine (1.2 eq) and N-hydroxybenzotriazole (1.2 eq) in DMA (0.5 M). The reaction mixture was stirred overnight at rt. Water (2×DMA volume) was added and obtained mixture was extracted with dichloromethane (3×DMA volume). The combined organic layers were washed with water, dried over Na₂SO₄, and concentrated under reduced pressure. The crude product was isolated by preparative HPLC or by column chromatography.

### Example Method 1: Formation of Chlorinated R^{x} Group of R^{x}COOH (or its Ester R^{x}COOR^{y})

NCS (1.1 eq) was added to a solution of an appropriate starting material (1 eq) in DMF (0.5 M) and the reaction mixture was stirred for 2 h at room temperature (20-25° C.). The reaction mixture was poured into water (2×DMF volume) and occurred precipitate was filtered. The solids were washed with water and dried in vacuum to give the acid, ROOH.

### Example Method 2: Synthesis of R^{x}COOH from Corresponding Ester R^{x}COOR^{y})

LiOH (1.1 eq) was added to a solution of an appropriate ester (1 eq) in THF:water mixture (3:1 or 5:1, 85 mM) and the resulting mixture was stirred overnight at room temperature (20-25° C.). The mixture was concentrated under reduced pressure, diluted with water, and acidified with concentrated HCl to pH=2-3. The precipitate was filtered, washed with water, and dried in vacuum to give the target carboxylic acid.

### Example Method 3: Formation of Acetylated R^{x} Group of R^{x}COOR^{y}

A mixture of an appropriate amine (1 eq.), Ac₂O (3 eq.), and DMAP (0.2 eq.) in dioxane (0.2 M) was heated to 80° C. for 2 h. Upon completion, the mixture was cooled down to room temperature (20-25° C.) and concentrated under reduced pressure. The residue was diluted with water (1× dioxane volume) and extracted with EtOAc (3× dioxane volume). The organic layers were washed with water, brine, dried over Na₂SO₄, and evaporated to dryness to afford an acylated product typically used without further purification.

Some examples of compounds of the present invention are shown below:

TABLE 1
example compounds of the invention

| | |
|---|---|
| 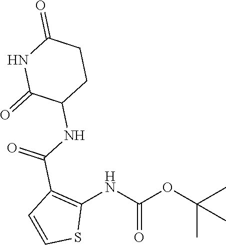 [SMILES: CC(C)(C)OC(=O)Nc1sccc1C(=O)NC1CCC(=O)NC1=O] | 1 |
| 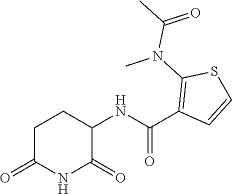 [SMILES: CC(=O)N(C)c1sccc1C(=O)NC1CCC(=O)NC1=O] | 2 |
| 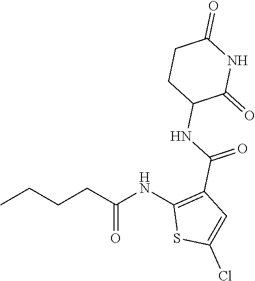 [SMILES: CCCCC(=O)Nc1sc(Cl)cc1C(=O)NC1CCC(=O)NC1=O] | 3 |
| 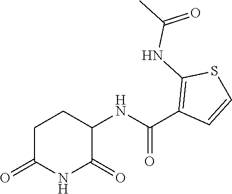 [SMILES: CC(=O)Nc1sccc1C(=O)NC1CCC(=O)NC1=O] | 4 |
| 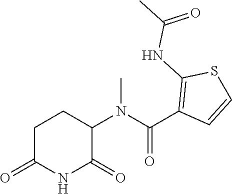 [SMILES: CC(=O)Nc1sccc1C(=O)N(C)C1CCC(=O)NC1=O] | 5 |
| 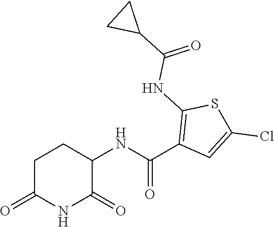 [SMILES: O=C1CCC(NC(=O)c2cc(Cl)sc2NC(=O)C2CC2)C(=O)N1] | 6 |
| 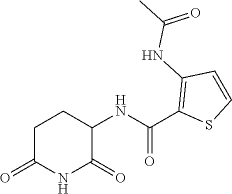 [SMILES: CC(=O)Nc1ccsc1C(=O)NC1CCC(=O)NC1=O] | 7 |
| 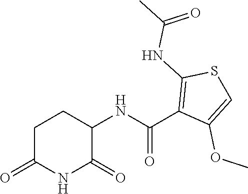 [SMILES: COc1csc(NC(C)=O)c1C(=O)NC1CCC(=O)NC1=O] | 8 |
| 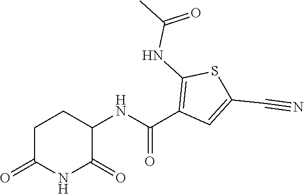 [SMILES: CC(=O)Nc1sc(C#N)cc1C(=O)NC1CCC(=O)NC1=O] | 9 |
| 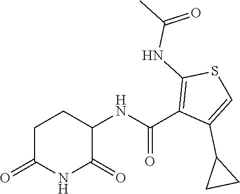 [SMILES: CC(=O)Nc1scc(C2CC2)c1C(=O)NC1CCC(=O)NC1=O] | 10 |
| 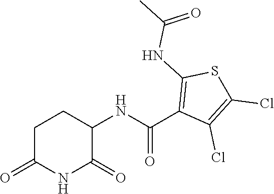 [SMILES: CC(=O)Nc1sc(Cl)c(Cl)c1C(=O)NC1CCC(=O)NC1=O] | 11 |
| 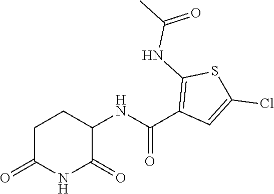 [SMILES: CC(=O)Nc1sc(Cl)cc1C(=O)NC1CCC(=O)NC1=O] | 12 |
| 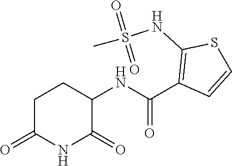 [SMILES: CS(=O)(=O)Nc1sccc1C(=O)NC1CCC(=O)NC1=O] | 13 |
| 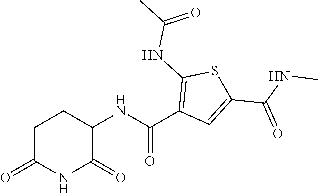 [SMILES: CNC(=O)c1cc(C(=O)NC2CCC(=O)NC2=O)c(NC(C)=O)s1] | 14 |
| 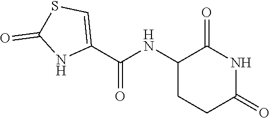 [SMILES: O=C1CCC(NC(=O)c2csc(=O)[nH]2)C(=O)N1] | 15 |
| 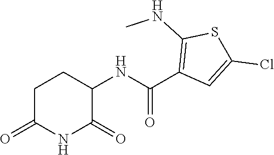 [SMILES: CNc1sc(Cl)cc1C(=O)NC1CCC(=O)NC1=O] | 16 |
| 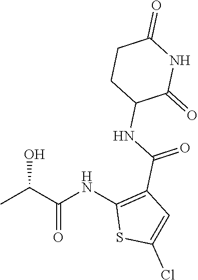 [SMILES: C[C@H](O)C(=O)Nc1sc(Cl)cc1C(=O)NC1CCC(=O)NC1=O] | 17 |
| 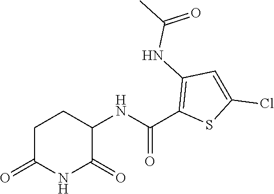 [SMILES: CC(=O)Nc1cc(Cl)sc1C(=O)NC1CCC(=O)NC1=O] | 18 |
| 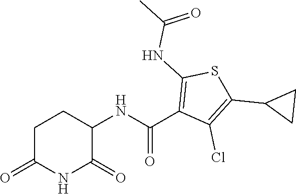 [SMILES: CC(=O)Nc1sc(C2CC2)c(Cl)c1C(=O)NC1CCC(=O)NC1=O] | 19 |
| 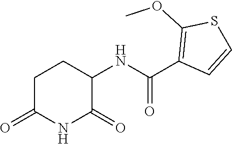 [SMILES: COc1sccc1C(=O)NC1CCC(=O)NC1=O] | 20 |
| 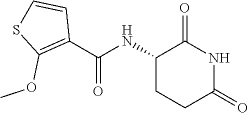 [SMILES: COc1sccc1C(=O)N[C@H]1CCC(=O)NC1=O] | 21 |
| 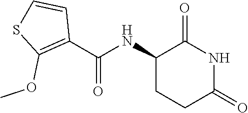 [SMILES: COc1sccc1C(=O)N[C@@H]1CCC(=O)NC1=O] | 22 |
| 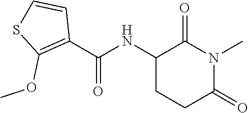 | 23 |
| 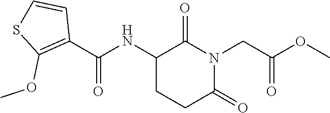 | 25 |
| 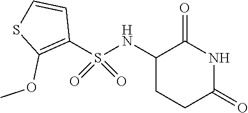 [SMILES: COc1sccc1S(=O)(=O)NC1CCC(=O)NC1=O] | 27 |
| 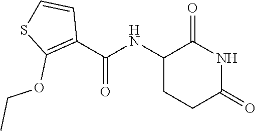 [SMILES: CCOc1sccc1C(=O)NC1CCC(=O)NC1=O] | 28 |
| 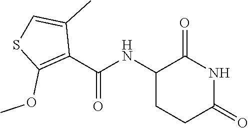 [SMILES: COc1scc(C)c1C(=O)NC1CCC(=O)NC1=O] | 29 |
| 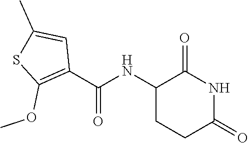 [SMILES: COc1sc(C)cc1C(=O)NC1CCC(=O)NC1=O] | 30 |
| 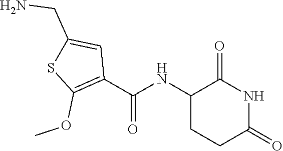 [SMILES: COc1sc(CN)cc1C(=O)NC1CCC(=O)NC1=O] | 31 |
| 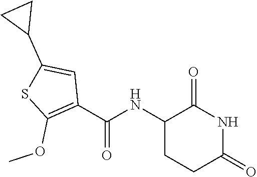 [SMILES: COc1sc(C2CC2)cc1C(=O)NC1CCC(=O)NC1=O] | 32 |
| 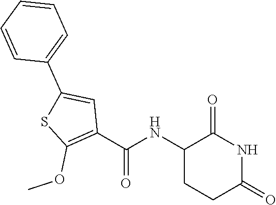 [SMILES: COc1sc(-c2ccccc2)cc1C(=O)NC1CCC(=O)NC1=O] | 33 |
| 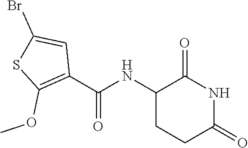 [SMILES: COc1sc(Br)cc1C(=O)NC1CCC(=O)NC1=O] | 34 |
| 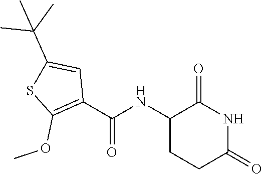 [SMILES: COc1sc(C(C)(C)C)cc1C(=O)NC1CCC(=O)NC1=O] | 35 |
| 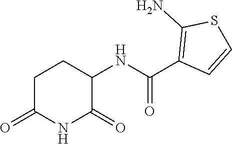 [SMILES: Nc1sccc1C(=O)NC1CCC(=O)NC1=O] | 36 |
| 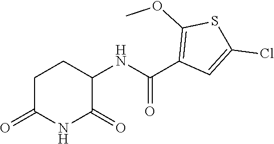 [SMILES: COc1sc(Cl)cc1C(=O)NC1CCC(=O)NC1=O] | 37 |
| 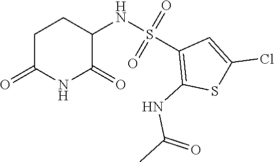 [SMILES: CC(=O)Nc1sc(Cl)cc1S(=O)(=O)NC1CCC(=O)NC1=O] | 38 |
| 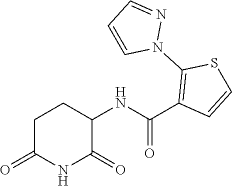 [SMILES: O=C1CCC(NC(=O)c2ccsc2-n2cccn2)C(=O)N1] | 39 |
| 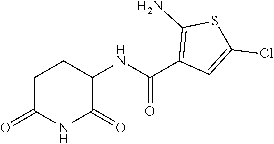 [SMILES: Nc1sc(Cl)cc1C(=O)NC1CCC(=O)NC1=O] | 40 |
| 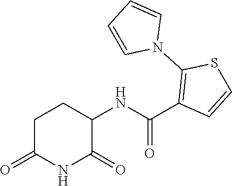 [SMILES: O=C1CCC(NC(=O)c2ccsc2-n2cccc2)C(=O)N1] | 41 |
| 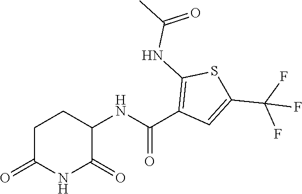 [SMILES: CC(=O)Nc1sc(C(F)(F)F)cc1C(=O)NC1CCC(=O)NC1=O] | 42 |
| 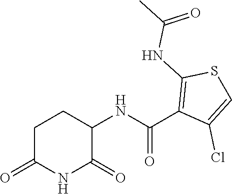 [SMILES: CC(=O)Nc1scc(Cl)c1C(=O)NC1CCC(=O)NC1=O] | 43 |
| 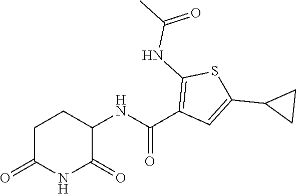 [SMILES: CC(=O)Nc1sc(C2CC2)cc1C(=O)NC1CCC(=O)NC1=O] | 44 |
| 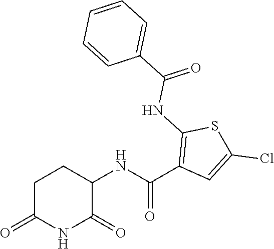 [SMILES: O=C1CCC(NC(=O)c2cc(Cl)sc2NC(=O)c2ccccc2)C(=O)N1] | 45 |
| 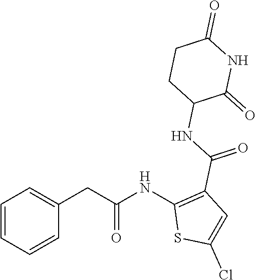 [SMILES: O=C1CCC(NC(=O)c2cc(Cl)sc2NC(=O)Cc2ccccc2)C(=O)N1] | 46 |
| 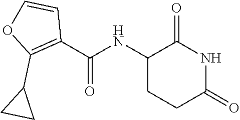 [SMILES: O=C1CCC(NC(=O)c2ccoc2C2CC2)C(=O)N1] | 47 |
| 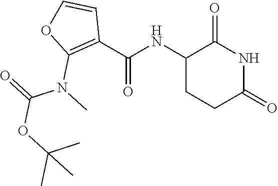 [SMILES: CN(C(=O)OC(C)(C)C)c1occc1C(=O)NC1CCC(=O)NC1=O] | 48 |
| 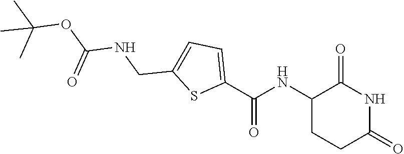 [SMILES: CC(C)(C)OC(=O)NCc1ccc(C(=O)NC2CCC(=O)NC2=O)s1] | 49 |
| 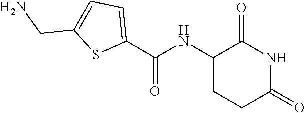 [SMILES: NCc1ccc(C(=O)NC2CCC(=O)NC2=O)s1] | 50 |
| 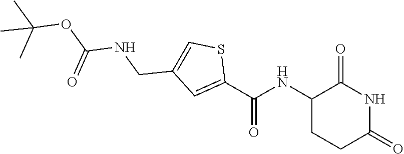 [SMILES: CC(C)(C)OC(=O)NCc1csc(C(=O)NC2CCC(=O)NC2=O)c1] | 51 |
| 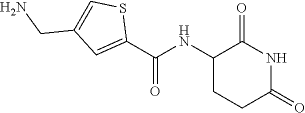 [SMILES: NCc1csc(C(=O)NC2CCC(=O)NC2=O)c1] | 52 |
| 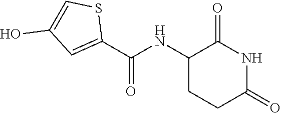 [SMILES: O=C1CCC(NC(=O)c2cc(O)cs2)C(=O)N1] | 53 |
| 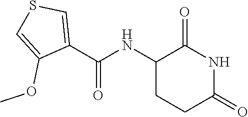 [SMILES: COc1cscc1C(=O)NC1CCC(=O)NC1=O] | 54 |
| 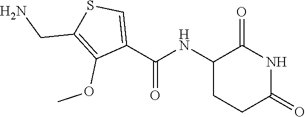 [SMILES: COc1c(C(=O)NC2CCC(=O)NC2=O)csc1CN] | 55 |
| 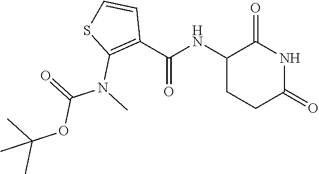 [SMILES: CN(C(=O)OC(C)(C)C)c1sccc1C(=O)NC1CCC(=O)NC1=O] | 56 |
| 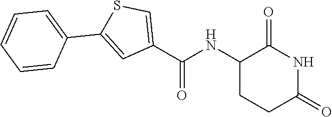 [SMILES: O=C1CCC(NC(=O)c2csc(-c3ccccc3)c2)C(=O)N1] | 57 |
| 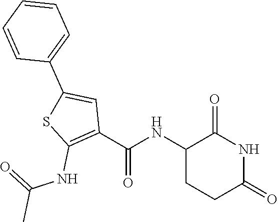 [SMILES: CC(=O)Nc1sc(-c2ccccc2)cc1C(=O)NC1CCC(=O)NC1=O] | 58 |
| 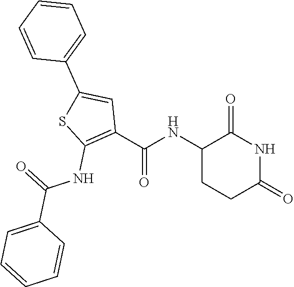 [SMILES: O=C1CCC(NC(=O)c2cc(-c3ccccc3)sc2NC(=O)c2ccccc2)C(=O)N1] | 59 |
| 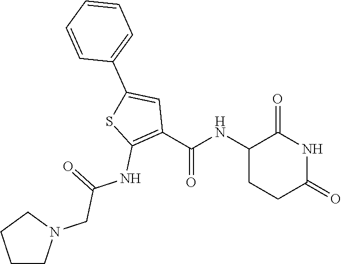 [SMILES: O=C1CCC(NC(=O)c2cc(-c3ccccc3)sc2NC(=O)CN2CCCC2)C(=O)N1] | 60 |
| 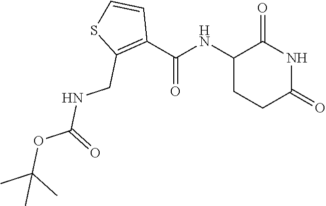 [SMILES: CC(C)(C)OC(=O)NCc1sccc1C(=O)NC1CCC(=O)NC1=O] | 63 |
| 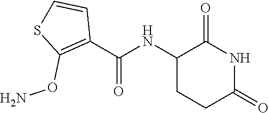 [SMILES: NOc1sccc1C(=O)NC1CCC(=O)NC1=O] | 64 |
| 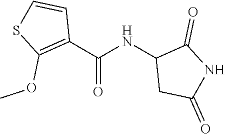 [SMILES: COc1sccc1C(=O)NC1CC(=O)NC1=O] | 65 |
| 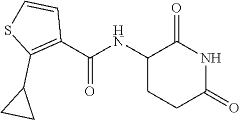 [SMILES: O=C1CCC(NC(=O)c2ccsc2C2CC2)C(=O)N1] | 66 |
| 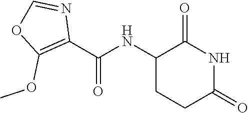 [SMILES: COc1ocnc1C(=O)NC1CCC(=O)NC1=O] | 67 |
| 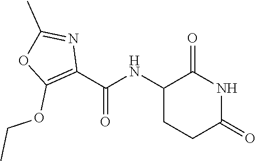 [SMILES: CCOc1oc(C)nc1C(=O)NC1CCC(=O)NC1=O] | 68 |
| 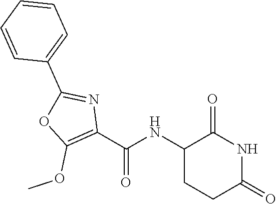 [SMILES: COc1oc(-c2ccccc2)nc1C(=O)NC1CCC(=O)NC1=O] | 69 |
| 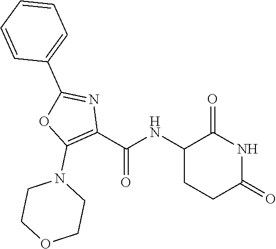 [SMILES: O=C1CCC(NC(=O)c2nc(-c3ccccc3)oc2N2CCOCC2)C(=O)N1] | 70 |
| 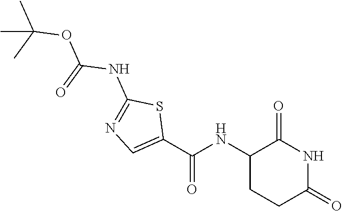 [SMILES: CC(C)(C)OC(=O)Nc1ncc(C(=O)NC2CCC(=O)NC2=O)s1] | 71 |
| 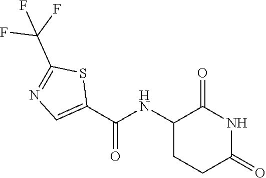 [SMILES: O=C1CCC(NC(=O)c2cnc(C(F)(F)F)s2)C(=O)N1] | 72 |
| 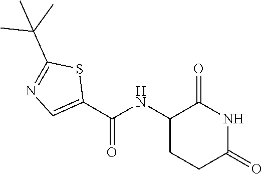 [SMILES: CC(C)(C)c1ncc(C(=O)NC2CCC(=O)NC2=O)s1] | 73 |
| 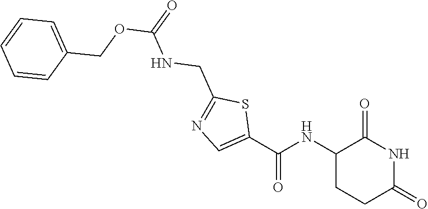 [SMILES: O=C1CCC(NC(=O)c2cnc(CNC(=O)OCc3ccccc3)s2)C(=O)N1] | 74 |
| 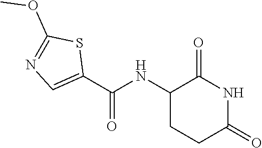 [SMILES: COc1ncc(C(=O)NC2CCC(=O)NC2=O)s1] | 75 |
| 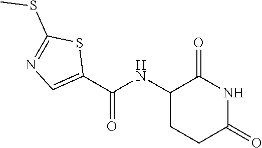 [SMILES: CSc1ncc(C(=O)NC2CCC(=O)NC2=O)s1] | 76 |
| 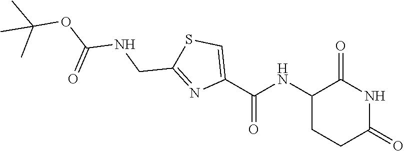 [SMILES: CC(C)(C)OC(=O)NCc1nc(C(=O)NC2CCC(=O)NC2=O)cs1] | 77 |
| 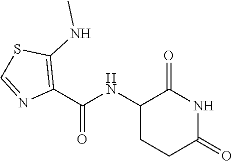 [SMILES: CNc1scnc1C(=O)NC1CCC(=O)NC1=O] | 78 |
| 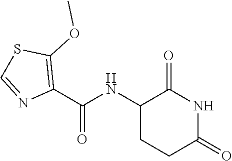 [SMILES: COc1scnc1C(=O)NC1CCC(=O)NC1=O] | 79 |
| 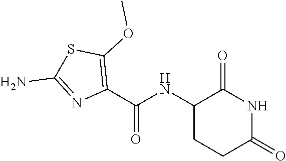 [SMILES: COc1sc(N)nc1C(=O)NC1CCC(=O)NC1=O] | 80 |
| 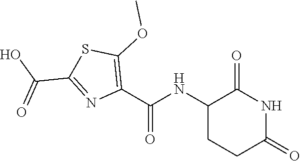 [SMILES: COc1sc(C(=O)O)nc1C(=O)NC1CCC(=O)NC1=O] | 81 |
| 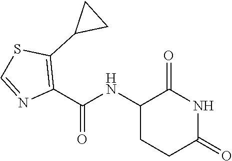 [SMILES: O=C1CCC(NC(=O)c2ncsc2C2CC2)C(=O)N1] | 82 |
| 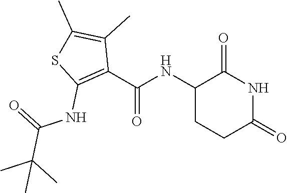 [SMILES: Cc1sc(NC(=O)C(C)(C)C)c(C(=O)NC2CCC(=O)NC2=O)c1C] | 83 |
| 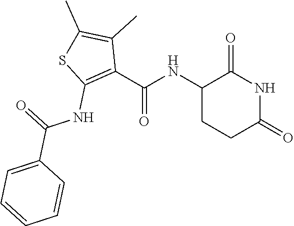 [SMILES: Cc1sc(NC(=O)c2ccccc2)c(C(=O)NC2CCC(=O)NC2=O)c1C] | 87 |
| 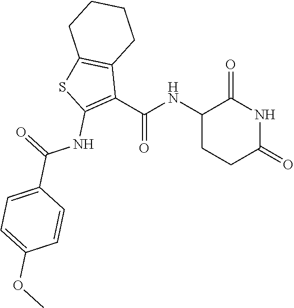 [SMILES: COc1ccc(C(=O)Nc2sc3c(c2C(=O)NC2CCC(=O)NC2=O)CCCC3)cc1] | 89 |
| 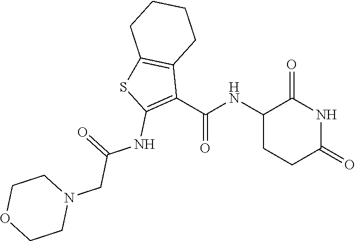 [SMILES: O=C1CCC(NC(=O)c2c(NC(=O)CN3CCOCC3)sc3c2CCCC3)C(=O)N1] | 90 |
| 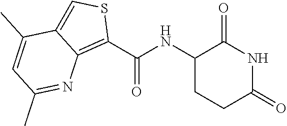 [SMILES: Cc1cc(C)c2csc(C(=O)NC3CCC(=O)NC3=O)c2n1] | 92 |
| 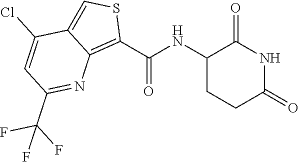 [SMILES: O=C1CCC(NC(=O)c2scc3c(Cl)cc(C(F)(F)F)nc23)C(=O)N1] | 93 |
| 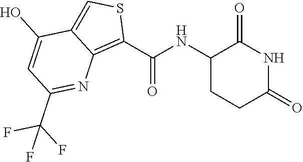 [SMILES: O=C1CCC(NC(=O)c2scc3c(O)cc(C(F)(F)F)nc23)C(=O)N1] | 94 |
| 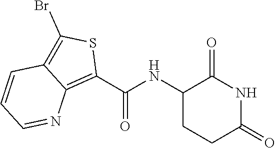 [SMILES: O=C1CCC(NC(=O)c2sc(Br)c3cccnc23)C(=O)N1] | 96 |
| 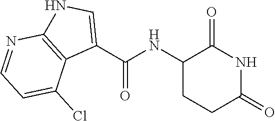 [SMILES: O=C1CCC(NC(=O)c2c[nH]c3nccc(Cl)c23)C(=O)N1] | 97 |
| 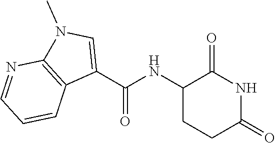 [SMILES: Cn1cc(C(=O)NC2CCC(=O)NC2=O)c2cccnc21] | 98 |
| 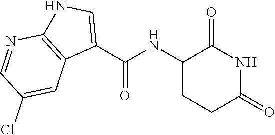 [SMILES: O=C1CCC(NC(=O)c2c[nH]c3ncc(Cl)cc23)C(=O)N1] | 99 |
| 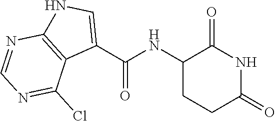 [SMILES: O=C1CCC(NC(=O)c2c[nH]c3ncnc(Cl)c23)C(=O)N1] | 100 |
| 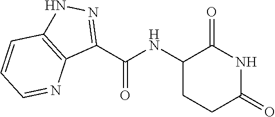 [SMILES: O=C1CCC(NC(=O)c2n[nH]c3cccnc23)C(=O)N1] | 101 |
| 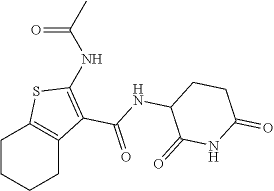 [SMILES: CC(=O)Nc1sc2c(c1C(=O)NC1CCC(=O)NC1=O)CCCC2] | 105 |
| 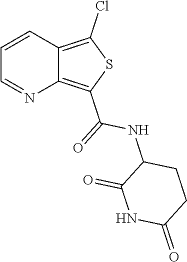 [SMILES: O=C1CCC(NC(=O)c2sc(Cl)c3cccnc23)C(=O)N1] | 106 |
| 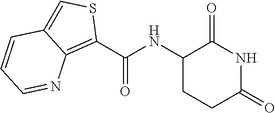 [SMILES: O=C1CCC(NC(=O)c2scc3cccnc23)C(=O)N1] | 107 |
| 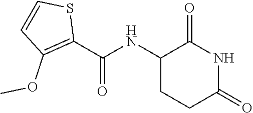 [SMILES: COc1ccsc1C(=O)NC1CCC(=O)NC1=O] | 108 |
| 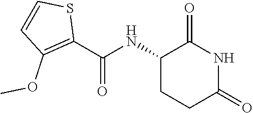 [SMILES: COc1ccsc1C(=O)N[C@H]1CCC(=O)NC1=O] | 109 |
| 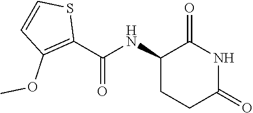 [SMILES: COc1ccsc1C(=O)N[C@@H]1CCC(=O)NC1=O] | 110 |
| 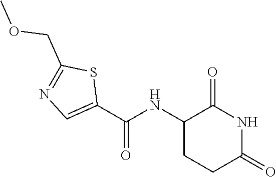 [SMILES: COCc1ncc(C(=O)NC2CCC(=O)NC2=O)s1] | 111 |
| 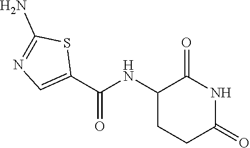 [SMILES: Nc1ncc(C(=O)NC2CCC(=O)NC2=O)s1] | 112 |

As also discussed in the Examples section, the present inventors have found that compounds of the present invention exhibits similar cereblon binding capabilities to that of the known CMA, CC-122. Despite the pharmaceutical activity of the known CMAs such as CC-122, patients often develop resistance to these compounds. The use of novel compounds—such as those of the present invention, as described above—may help to overcome this clinical obstacle.

One of the serious disadvantages of the currently available CMAs is their safety profile. For example, the teratogenicity of the CMAs is dependent upon the extent to which the CMAs induce degradation of SALL4 transcription factor. Known CMAs induce degradation of several proteins (including SALL4) which bind to CRL4^{CRBN} ligase only in presence of the CMA. SALL4 degradation, observed under treatment with CMAs, is responsible (at least partly) for the teratogenicity of the CMAs. Compounds with diminished capability to induce SALL4 degradation may demonstrate an improved safety profile.

The compounds of the present invention may also possess pharmaceutically advantageous properties, such as increased stability and improved ADMET (absorption, distribution, metabolism, excretion, and/or toxicity) properties.

The compounds of the present invention may be useful in the treatment of various diseases and disorders, including (but not limited to):
1) Cancer. The compounds provided herein can be used for treating, preventing or managing either primary or metastatic tumors. Specific examples of cancer include, but are not limited to, cancers of the skin, such as melanoma; lymph node; breast; cervix; uterus; gastrointestinal tract; lung; ovary; prostate; colon; rectum; mouth; brain; head and neck; throat; testes; kidney; pancreas; bone; spleen; liver; bladder; larynx; nasal passages, and AIDS-related cancers and hematological malignancies.
   a) Hematological malignancies include leukemia, lymphoma, multiple myeloma or smoldering myeloma.
      Leukemia can be selected from: acute leukemia, acute lymphoblastic leukemia (ALL), chronic lymphocytic leukemia (CLL), acute myelogenous leukemia, acute myeloid leukemia (AML), adult acute basophilic leukemia, adult acute eosinophilic leukemia, adult acute megakaryoblastic leukemia, adult acute minimally differentiated myeloid leukemia, adult acute monoblastic leukemia, adult acute monocytic leukemia, adult acute myeloblastic leukemia with maturation, adult acute myeloblastic leukemia without maturation, adult acute myeloid leukemia with abnormalities, adult acute myelomonocytic leukemia, adult erythroleukemia, adult pure erythroid leukemia, secondary acute myeloid leukemia, untreated adult acute myeloid leukemia, adult acute myeloid leukemia in remission, adult acute promyelocytic leukemia with PML-RARA, alkylating agent-related acute myeloid leukemia, prolymphocytic leukemia, and chronic myelomonocytic leukemia, refractory hairy cell leukemia, T-cell large granular lymphocyte leukemia, relapsed or refractory chronic lymphocytic leukemia.
      Lymphoma can be selected from the group consisting of: adult grade Ill lymphomatoid granulomatosis, adult nasal type extranodal NK/T-cell lymphoma, anaplastic large cell lymphoma, angioimmunoblastic T-cell lymphoma, cutaneous B-Cell non-Hodgkin lymphoma, extranodal marginal zone lymphoma of mucosa-associated lymphoid tissue, hepatosplenic T-cell lymphoma, intraocular lymphoma, lymphomatous involvement of non-cutaneous extranodal site, mature T-cell and K-cell non-Hodgkin lymphoma, nodal marginal zone lymphoma, post-transplant lymphoproliferative disorder, recurrent adult Burkitt lymphoma, recurrent adult diffuse large cell lymphoma, recurrent adult diffuse mixed cell lymphoma, recurrent adult diffuse small cleaved cell lymphoma, recurrent adult grade Ill lymphomatoid granulomatosis, recurrent adult immunoblastic lymphoma, recurrent adult lymphoblastic lymphoma, recurrent adult T-cell leukemia/lymphoma, recurrent cutaneous T-cell non-Hodgkin lymphoma, recurrent grade 1 follicular lymphoma, recurrent grade 2 follicular lymphoma, recurrent grade 3 follicular lymphoma, recurrent mantle cell lymphoma, recurrent marginal zone lymphoma, recurrent mycosis fungoides and Sezary syndrome, recurrent small lymphocytic lymphoma, Richter syndrome, small intestinal lymphoma, splenic marginal zone lymphoma, testicular lymphoma, Waldenstrom macroglobulinemia, adult T-cell leukemia-lymphoma, peripheral T-cell lymphoma, B-cell lymphoma, Hodgkin's disease, cutaneous T-cell lymphoma, diffuse large B-cell lymphoma, MALT lymphoma, mantle cell lymphoma, non-Hodgkins lymphoma, refractory primary-cutaneous large B-cell lymphoma (Leg-type), refractory anemia, refractory anemia with excess blasts, refractory anemia with ringed sideroblasts, refractory cytopenia with multilineage dysplasia, secondary myelodysplastic syndromes, myelodysplastic syndrome, and myeloproliferative disease.
2) Autoimmune diseases, such as: Acute disseminated encephalomyelitis, acute motor axonal neuropathy, Addison's disease, adiposis dolorosa, adult-onset Still's disease, alopecia areata, ankylosing spondylitis, anti-glomerular basement membrane nephritis, anti-neutrophil cytoplasmic antibody-associated vasculitis, anti-N-methyl-D-aspartate receptor encephalitis, antiphospholipid syndrome, antisynthetase syndrome, aplastic anemia, autoimmune angioedema, autoimmune encephalitis, autoimmune enteropathy, autoimmune hemolytic anemia, autoimmune hepatitis, autoimmune inner ear disease, autoimmune lymphoproliferative syndrome, autoimmune neutropenia, autoimmune oophoritis, autoimmune orchitis, autoimmune pancreatitis, autoimmune polyendocrine syndrome, autoimmune polyendocrine syndrome type 2, autoimmune polyendocrine syndrome type 3, autoimmune progesterone dermatitis, autoimmune retinopathy, autoimmune thrombocytopenic purpura, autoimmune thyroiditis, autoimmune urticaria, autoimmune uveitis, balo concentric sclerosis, Behcet's disease, Bickerstaff's encephalitis, bullous pemphigoid, celiac disease, chronic fatigue syndrome, chronic inflammatory demyelinating polyneuropathy, churg-Strauss syndrome, cicatricial pemphigoid, cogan syndrome, cold agglutinin disease, complex regional pain syndrome, CREST syndrome, Crohn's disease, dermatitis herpetiformis, dermatomyositis, diabetes mellitus type 1, discoid lupus erythematosus, endometriosis, enthesitis, enthesitis-related arthritis, eosinophilic esophagitis, eosinophilic fasciitis, epidermolysis bullosa acquisita, erythema nodosum. essential mixed cryoglobulinemia, evans syndrome, felty syndrome, fibromyalgia, gastritis, gestational pemphigoid, giant cell arteritis, goodpasture syndrome, Graves' disease, graves ophthalmopathy, Guillain-Barré syndrome, hashimoto's encephalopathy, hashimoto thyroiditis, Henoch-Schonlein purpura, hidradenitis suppurativa, idiopathic inflammatory demyelinating diseases, IgG4-related systemic disease, inclusion body myositis, inflamatory bowel disease (IBD), intermediate uveitis, interstitial cystitis, juvenile arthritis, kawasaki's disease, Lambert-Eaton myasthenic syndrome, leukocytoclastic vasculitis, Lichen planus, Lichen sclerosus, ligneous conjunctivitis, linear IgA disease, lupus nephritis, lupus vasculitis, lyme disease (Chronic), Meniere's disease, microscopic colitis, microscopic polyangiitis, mixed connective tissue disease, Mooren's ulcer, morphea, Mucha-Habermann disease, multiple sclerosis, myasthenia gravis, myocarditis, myositis, neuromyelitis optica, neuromyotonia, opsoclonus myoclonus syndrome, optic neuritis, Ord's thyroiditis, palindromic rheumatism, paraneoplastic cerebellar degeneration, Parry Romberg syndrome, Parsonage-Turner syndrome, pediatric autoimmune neuropsychiatric disorder associated with streptococcus, pemphigus vulgaris, pernicious anemia, pityriasis lichenoides et varioliformis acuta, POEMS syndrome, polyarteritis nodosa, polymyalgia rheumatica, polymyositis, postmyocardial infarction syndrome, postpericardiotomy syndrome, primary biliary cirrhosis, primary immunodeficiency, primary sclerosing cholangitis, progressive inflammatory neuropathy, psoriasis, psoriatic arthritis, pure red cell aplasia, pyoderma gangrenosum, Raynaud's phenomenon, reactive arthritis, relapsing polychondritis, restless leg syndrome, retroperitoneal fibrosis, rheumatic fever, rheumatoid arthritis, rheumatoid vasculitis, sarcoidosis, Schnitzler syndrome, scleroderma, Sjogren's syndrome, stiff person syndrome, subacute bacterial endocarditis, Susac's syndrome, Sydenham chorea, sympathetic ophthalmia, systemic lupus erythematosus, systemic scleroderma, thrombocytopenia, Tolosa-Hunt syndrome, transverse myelitis, ulcerative colitis, undifferentiated connective tissue disease, urticaria, urticarial vasculitis, vasculitis and vitiligo;
3) Diseases and disorders associated with, or characterized by, undesired angiogenesis, including inflammatory diseases, autoimmune diseases, pain, viral diseases, genetic diseases, allergic diseases, bacterial diseases, ocular neovascular diseases, choroidal neovascular diseases, retina neovascular diseases, and rubeosis (neovascularization of the angle). Specific examples of the diseases and disorders associated with, or characterized by, undesired angiogenesis include, but are not limited to: arthritis, endometriosis, Crohn's disease, heart failure, advanced heart failure, renal impairment, endotoxemia, toxic shock syndrome, osteoarthritis, retrovirus replication, wasting, meningitis, silica-induced fibrosis, asbestos-induced fibrosis, veterinary disorder, malignancy-associated hypercalcemia, stroke, circulatory shock, periodontitis, gingivitis, macrocytic anemia, refractory anemia, and 5q-deletion syndrome, nociceptive pain, neuropathic pain, mixed pain of nociceptive and neuropathic pain, visceral pain, migraine, headache and postoperative pain. Examples of nociceptive pain include, but are not limited to, pain associated with chemical or thermal burns, cuts of the skin, contusions of the skin, osteoarthritis, rheumatoid arthritis, tendonitis, and myofascial pain. Examples of neuropathic pain include, but are not limited to, CRPS type I, CRPS type II, reflex sympathetic dystrophy (RSD), reflex neurovascular dystrophy, reflex dystrophy, sympathetically maintained pain syndrome, causalgia, Sudeck atrophy of bone, algoneurodystrophy, shoulder hand syndrome, post-traumatic dystrophy, trigeminal neuralgia, post herpetic neuralgia, cancer related pain, phantom limb pain, fibromyalgia, chronic fatigue syndrome, spinal cord injury pain, central post-stroke pain, radiculopathy, diabetic neuropathy, post-stroke pain, luetic neuropathy, and other painful neuropathic conditions such as those induced by drugs such as vincristine and velcade;
4) Macular Degeneration (“MD”) and related syndromes, such as: atrophic (dry) MD, exudative (wet) MD, age-related maculopathy (ARM), choroidal neovascularisation (CNVM), retinal pigment epithelium detachment (PED), and atrophy of retinal pigment epithelium (RPE);
5) Skin diseases such as: keratoses and related symptoms, skin diseases or disorders characterized with overgrowths of the epidermis, acne, and wrinkles. Examples of skin diseases or disorders characterized with overgrowths of the epidermis include, but are not limited to, any conditions, diseases or disorders marked by the presence of overgrowths of the epidermis, including but not limited to, infections associated with papilloma virus, arsenical keratoses, sign of Leser-Trélat, warty dyskeratoma (WD), trichostasis spinulosa (TS), erythrokeratodermia variabilis (EKV), ichthyosis fetalis (harlequin ichthyosis), knuckle pads, cutaneous melanoacanthoma, porokeratosis, psoriasis, squamous cell carcinoma, confluent and reticulated papillomatosis (CRP), acrochordons, cutaneous horn, cowden disease (multiple hamartoma syndrome), dermatosis papulosa nigra (DPN), epidermal nevus syndrome (ENS), ichthyosis vulgaris, molluscum contagiosum, prurigo nodularis, and acanthosis nigricans (AN);
6) Pulmonary disorders, such as pulmonary hypertension and related disorders. Examples of pulmonary hypertension and related disorders include, but are not limited to: primary pulmonary hypertension (PPH); secondary pulmonary hypertension (SPH); familial PPH; sporadic PPH; precapillary pulmonary hypertension; pulmonary arterial hypertension (PAH); pulmonary artery hypertension; idiopathic pulmonary hypertension; thrombotic pulmonary arteriopathy (TPA); plexogenic pulmonary arteriopathy; functional classes I to IV pulmonary hypertension; and pulmonary hypertension associated with, related to, or secondary to, left ventricular dysfunction, mitral valvular disease, constrictive pericarditis, aortic stenosis, cardiomyopathy, mediastinal fibrosis, anomalous pulmonary venous drainage, pulmonary venoocclusive disease, collagen vasular disease, congenital heart disease, HIV virus infection, drugs and toxins such as fenfluramines, congenital heart disease, pulmonary venous hypertension, chronic obstructive pulmonary disease, interstitial lung disease, sleep-disordered breathing, alveolar hypoventilation disorder, chronic exposure to high altitude, neonatal lung disease, alveolar-capillary dysplasia, sickle cell disease, other coagulation disorder, chronic thromboemboli, connective tissue disease, lupus including systemic and cutaneous lupus, schistosomiasis, sarcoidosis or pulmonary capillary hemangiomatosis;
7) Asbestos-related disorders, such as: mesothelioma, asbestosis, malignant pleural effusion, benign exudative effusion, pleural plaques, pleural calcification, diffuse pleural thickening, rounded atelectasis, fibrotic masses, and lung cancer;
8) Parasitic diseases and disorders caused by human intracellular parasites such as, but not limited to, P. falcifarium, P. ovale, P. vivax, P. malariae, L. donovari, L. infanium, L. aethiopica, L. major, L. tropica, L mexicana, L braziliensis, T. Gondii, B. microti, B. divergens, B. coli, C. parvum, C. cayetanensis, E. histolytica, I. belli, S. monsonii, S. haemolobium, Trypanosoma ssp., Toxoplasma ssp., and O. volvulus. Other diseases and disorders caused by non-human intracellular parasites such as, but not limited to, Babesia bovis, Babesia canis, Banesia Gibsoni, Besnoitia darlingi, Cytauxzoon felis, Eimeria ssp., Hammondia ssp., and Theileria ssp., are also encompassed. Specific examples include, but are not limited to, malaria, babesiosis, trypanosomiasis, leishmaniasis, toxoplasmosis, meningoencephalitis, keratitis, amebiasis, giardiasis, cryptosporidiosis, isosporiasis, cyclosporiasis, microsporidiosis, ascariasis, trichuriasis, ancylostomiasis, strongyloidiasis, toxocariasis, trichinosis, lymphatic filariasis, onchocerciasis, filariasis, schistosomiasis, and dermatitis caused by animal schistosomes;
9) Immunodeficiency disorders, which include, but are not limited to, adenosine deaminase deficiency, antibody deficiency with normal or elevated Igs, ataxia-tenlangiectasia, bare lymphocyte syndrome, common variable immunodeficiency, Ig deficiency with hyper-IgM, Ig heavy chain deletions, IgA deficiency, immunodeficiency with thymoma, reticular dysgenesis, Nezelof syndrome, selective IgG subclass deficiency, transient hypogammaglobulinemia of infancy, Wistcott-Aldrich syndrome, X-linked agammaglobulinemia, X-linked severe combined immunodeficiency;
10) Atherosclerosis and related conditions, such as: all forms of conditions involving atherosclerosis, including restenosis after vascular intervention such as angioplasty, stenting, atherectomy and grafting;
11) Hemoglobinopathy and related disorders, such as sickle cell anemia, and any other disorders related to the differentiation of CD34+ cells;
12) TNFα related disorders, such as: endotoxemia or toxic shock syndrome; cachexia; adult respiratory distress syndrome; bone resorption diseases such as arthritis; hypercalcemia; Graft versus Host Reaction; cerebral malaria; inflammation; tumor growth; chronic pulmonary inflammatory diseases; reperfusion injury; myocardial infarction; stroke; circulatory shock; rheumatoid arthritis; Crohn's disease; HIV infection and AIDS; other disorders such as rheumatoid arthritis, rheumatoid. spondylitis, osteoarthritis, psoriatic arthritis and other arthritic conditions, septic shock, septis, endotoxic shock, graft versus host disease, wasting, Crohn's disease, ulcerative colitis, multiple sclerosis, systemic lupus erythromatosis, ENL in leprosy, HIV, AIDS, and opportunistic infections in AIDS; disorders such as septic shock, sepsis, endotoxic shock, hemodynamic shock and sepsis syndrome, post ischemic reperfusion injury, malaria, mycobacterial infection, meningitis, psoriasis, congestive heart failure, fibrotic disease, cachexia, graft rejection, oncogenic or cancerous conditions, asthma, autoimmune disease, radiation damages, and hyperoxic alveolar injury; viral infections, such as those caused by the herpes viruses; viral conjunctivitis; or atopic dermatitis;

The compounds of the present invention may also be useful in preventing, treating, or reducing the risk of developing graft versus host disease (GVHD) or transplant rejection.

The compounds of the present invention may also inhibit the production of certain cytokines including, but not limited to, TNF-α, IL-1β, IL-12, IL-18, GM-CSF, IL-10, TGF-β and/or IL-6. The present compounds may stimulate the production of certain cytokines, and also act as a costimulatory signal for T cell activation, resulting in increased production of cytokines such as, but not limited to, IL-12, IL-2, IL-10, TGF-β and/or IFN-γ. In addition, compounds provided herein can enhance the effects of NK cells and antibody-mediated cellular cytotoxicity (ADCC). Further, compounds provided herein may be immunomodulatory and/or cytotoxic, and thus may be useful as chemotherapeutic agents.

### EXAMPLES

The compounds of the present invention are advantageous in terms of their synthetic feasibility. The synthesis of the compounds can be summarized in the following general procedure (carried out under Synthetic Conditions A or Synthetic Conditions B, as set out below:

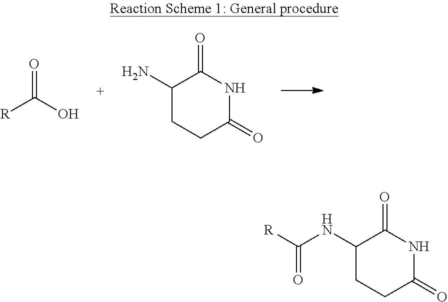

Synthetic Conditions A

An appropriate acid (RCOOH in the above reaction scheme) (1.1 eq), DMAP (0.04 eq), and EDC (1.2 eq) were added to a solution of 3-aminopiperidine-2,6-dione (1 eq) and N-hydroxybenzotriazole (1.2 eq) in DMF (0.5 M). The reaction mixture was stirred overnight at room temperature (20-25° C.). Water (2×DMF volume) was added and the obtained solution was extracted with dichloromethane (3×DMF volume). The combined organic layers were washed with water, dried over Na₂SO₄, and concentrated under reduced pressure. The crude product was purified by preparative HPLC or by column chromatography.

Synthetic Conditions B

An appropriate acid RCOOH in the above reaction scheme) (1 eq) and EDC (1.2 eq) were added to a solution of 3-aminopiperidine-2,6-dione (hydrochloride salt, 1.1 eq), triethylamine (1.2 eq) and N-hydroxybenzotriazole (1.2 eq) in DMA (0.5 M). The reaction mixture was stirred overnight at rt. Water (2×DMA volume) was added and obtained mixture was extracted with dichloromethane (3×DMA volume). The combined organic layers were washed with water, dried over Na₂SO₄, and concentrated under reduced pressure. The crude product was isolated by preparative HPLC or by column chromatography.

Synthetic Conditions C

To a solution of appropriate acid (RCOOH in the above reaction scheme) (1 eq) and HATU (1.5 eq) in dry DMF were added 3-aminopiperidine-2,6-dione (hydrochloride salt, 1.2 eq) and DIPEA (3 eq). The reaction mixture was stirred overnight at rt. The crude product was purified by preparative HPLC or/and by preparative TLC.

Synthetic Conditions D

To a solution of appropriate acid (RCOOH in the above reaction scheme) (1 eq) 3-aminopiperidine-2,6-dione (hydrochloride salt, 1.2 eq) and DMAP (0.1 eq.) in an inert atmosphere in dry DMF were added DIPEA (2.2 eq.) and HATU (1.5 eq) in dry DMF. The reaction mixture was stirred overnight at rt. The crude product was purified by preparative HPLC or/and by preparative TLC.

### Example Method 1: Formation of Chlorinated R Group of RCOOH (or its Ester RCOOR′)

N-chlorosuccinimide (1.1 eq) was added to a solution of an appropriate starting material (1 eq) in DMF (0.5 M) and the reaction mixture was stirred for 2 h at room temperature (20-25° C.). The reaction mixture was poured into water (2×DMF volume) and occurred precipitate was filtered. The solids were washed with water and dried in vacuum to give the acid, ROOH.

### Example Method 2: Synthesis of RCOOH from Corresponding Ester RCOOR′)

LiOH (1.1 eq) was added to a solution of an appropriate ester (1 eq) in THF:water mixture (3:1 or 5:1, 85 mM) and the resulting mixture was stirred overnight at room temperature (20-25° C.). The mixture was concentrated under reduced pressure, diluted with water, and acidified with concentrated HCl to pH=2-3. The precipitate was filtered, washed with water, and dried in vacuum to give the target carboxylic acid.

### Example Method 3: Formation of Acetylated R Group of RCOOR′

A mixture of an appropriate amine (1 eq.), Ac₂O (3 eq.), and DMAP (0.2 eq.) in dioxane (0.2 M) was heated to 80° C. for 2 h. Upon completion, the mixture was cooled down to room temperature (20-25° C.) and concentrated under reduced pressure. The residue was diluted with water (1× dioxane volume) and extracted with EtOAc (3× dioxane volume). The organic layers were washed with water, brine, dried over Na₂SO₄, and evaporated to dryness to afford an acylated product typically used without further purification.

### Example 1: Synthesis of tert-butyl (3-((2,6-dioxopiperidin-3-yl)carbamoyl)thiophen-2-yl)carbamate (1)

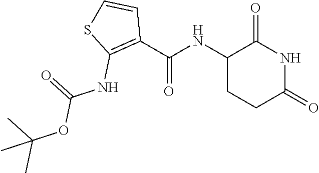 [SMILES: CC(C)(C)OC(=O)Nc1sccc1C(=O)NC1CCC(=O)NC1=O]

This compound was synthesized using the general procedure shown in Reaction Scheme 1 and Synthetic Conditions C, above, (52% yield) using 2-((tert-butoxycarbonyl)amino)thiophene-3-carboxylic acid (43 mg) as a starting material.

¹H NMR (500 MHz, DMSO) δ 11.09 (s, 1H), 10.88 (s, 1H), 8.63 (d, J=8.2 Hz, 1H), 7.38 (d, J=5.9 Hz, 1H), 6.97 (dd, J=5.8, 0.8 Hz, 1H), 4.68 (ddd, J=13.0, 8.2, 5.3 Hz, 1H), 2.78 (ddd, J=17.3, 13.5, 5.5 Hz, 1H), 2.59-2.54 (m, 1H), 2.15 (qd, J=13.0, 4.5 Hz, 1H), 1.96 (ddt, J=10.2, 5.3, 2.7 Hz, 1H), 1.48 (s, 9H).

LCMS (m/z [M+H]⁻): 352.0

### Example 2: Synthesis of N-(2,6-dioxopiperidin-3-yl)-2-(N-methylacetamido)thiophene-3-carboxamide (2)

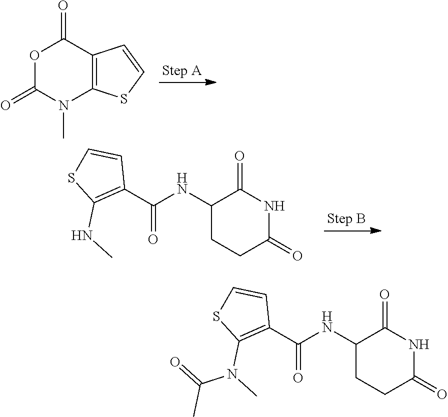

Step A: 3-Aminopiperidine-2,6-dione (3.3 g, 25.8 mmol) and triethylamine (2.45 g, 24.2 mmol) were added to a solution of 1-methyl-1H,2H,4H-thieno[2,3-d][1,3]oxazine-2,4-dione (3.7 g, 20.2 mmol) in ethanol (20 mL). The reaction mixture was refluxed for 16 h and filtered. The precipitate was washed with water to give N-(2,6-dioxopiperidin-3-yl)-2-(methylamino)thiophene-3-carboxamide (19% yield).

Step B: Acetic anhydride (0.265 g, 2.60 mmol) and DMAP (0.026 g, 0.213 mmol) were added to a solution of N-(2,6-dioxopiperidin-3-yl)-2-(methylamino)thiophene-3-carboxamide (0.579 g, 2.17 mmol) and triethylamine (0.263 g, 2.60 mmol) in dioxane (10 mL). The reaction mixture was stirred at 60° C. for 16 h, washed with water and extracted with EtOAc (3×10 mL), dried over Na₂SO₄, concentrated under reduced pressure and purified by HPLC to give N-(2,6-dioxopiperidin-3-yl)-2-(N-methylacetamido)thiophene-3-carboxamide (11% yield).

¹H NMR (500 MHz, DMSO) δ 10.86 (s, 1H), 8.53 (d, J=8.0 Hz, 1H), 7.56 (d, J=5.7 Hz, 1H), 7.34 (d, J=5.7 Hz, 1H), 4.72-4.62 (m, 1H), 3.09 (s, 3H), 2.83-2.71 (m, 1H), 2.58-2.53 (m, 1H), 2.19-2.02 (m, 1H), 1.99-1.90 (m, 1H), 1.81 (s, 3H).

LCMS (m/z [M+H]⁺): 310.2

### Example 3: Synthesis of 5-chloro-N-(2,6-dioxopiperidin-3-yl)-2-pentanamidothiophene-3-carboxamide (3)

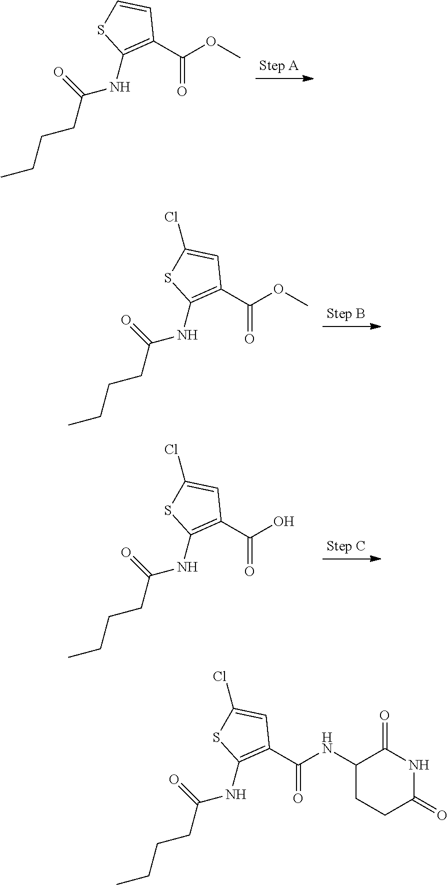 [SMILES: CCCCC(=O)Nc1sccc1C(=O)OC ; CCCCC(=O)Nc1sc(Cl)cc1C(=O)OC ; CCCCC(=O)Nc1sc(Cl)cc1C(=O)O ; CCCCC(=O)Nc1sc(Cl)cc1C(=O)NC1CCC(=O)NC1=O]

Step A: Methyl 5-chloro-2-pentanamidothiophene-3-carboxylate was synthesized using Example Method 1, above (65% yield), using methyl 2-pentanamidothiophene-3-carboxylate as a starting material.

Step B: 5-chloro-2-pentanamidothiophene-3-carboxylic acid was synthesized using Example

Method 2, above (69% yield), using methyl 5-chloro-2-pentanamidothiophene-3-carboxylate as a starting material.

Step C: 5-chloro-N-(2,6-dioxopiperidin-3-yl)-2-pentanamidothiophene-3-carboxamide was synthesized using the general procedure shown in Reaction Scheme 1 and Synthetic Conditions A, above (40% yield), and 5-chloro-2-pentanamidothiophene-3-carboxylic acid as a starting material.

¹H NMR (400 MHz, DMSO) δ 11.94 (s, 1H), 10.92 (s, 1H), 8.72-8.59 (m, 1H), 7.52 (s, 1H), 4.84-4.71 (m, 1H), 2.96-2.59 (m, 4H), 2.61-2.53 (m, 1H), 2.23-2.06 (m, 1H), 2.04-1.91 (m, 2H), 1.68-1.51 (m, 2H), 1.42-1.26 (m, 2H), 1.02-0.81 (m, 3H).

LCMS (m/z [M+H]⁺): 372.2

### Example 4: Synthesis of N-(2,6-dioxopiperidin-3-yl)-2-acetamidothiophene-3-carboxamide (4)

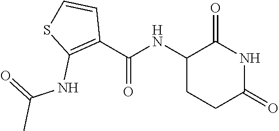 [SMILES: CC(=O)Nc1sccc1C(=O)NC1CCC(=O)NC1=O]

This compound was synthesized using the general procedure shown in Reaction Scheme 1 and Synthetic Conditions B, above, (32% yield) with 2-acetamidothiophene-3-carboxylic acid as a starting material.

¹H NMR (400 MHz, DMSO) δ 11.84 (s, 1H), 10.90 (s, 1H), 8.67 (d, J=8.1 Hz, 1H), 7.42 (d, J=5.8 Hz, 1H), 7.00 (d, J=5.7 Hz, 1H), 4.79-4.67 (m, 1H), 2.85-2.72 (m, 1H), 2.61-2.54 (m, 1H), 2.20 (s, 3H), 2.17-2.11 (m, 1H), 1.96-1.84 (m, 1H).

LCMS (m/z [M+H]⁺): 296.06

### Example 5: Synthesis of N-(2,6-dioxopiperidin-3-yl)-2-acetamido-N-methylthiophene-3-carboxamide (5)

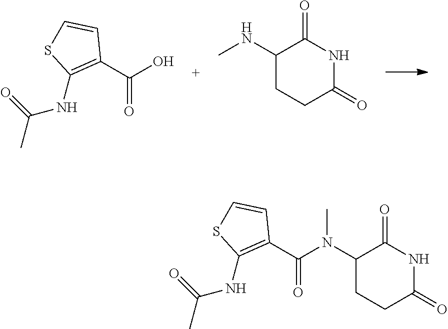

Triethylamine (0.236 g, 2.328 mmol), N-hydroxybenzotriazole (0.314, 2.3 mmol), 2-acetamidothiophene-3-carboxylic acid (0.359 g, 1.94 mmol) and EDC (0.361 g, 2.328 mmol) were added sequentially to a solution of 3-(methylamino)piperidine-2,6-dione (0.381 g, 2.134 mmol, hydrochloride salt) in DMA (30 mL) and the reaction mixture was stirred overnight at room temperature. Water (10 mL) was added and obtained solution was extracted with DCM, dried over Na₂SO₄ and concentrated under reduced pressure. The product was purified by HPLC to give N-(2,6-dioxopiperidin-3-yl)-2-acetamido-N-methylthiophene-3-carboxamide (25% yield).

¹H NMR (400 MHz, DMSO) δ 10.99 (s, 1H), 10.54 (s, 1H), 7.13-7.02 (m, 1H), 7.01-6.89 (m, 1H), 5.13-4.87 (m, 1H), 2.87 (s, 3H), 2.83-2.71 (m, 1H), 2.61-2.52 (m, 2H), 2.15 (s, 3H), 2.07-1.95 (m, 1H).

LCMS (m/z [M+H]⁺): 310.0

### Example 6: Synthesis of 5-chloro-2-cyclopropaneamido-N-(2,6-dioxopiperidin-3-yl)thiophene-3-carboxamide (6)

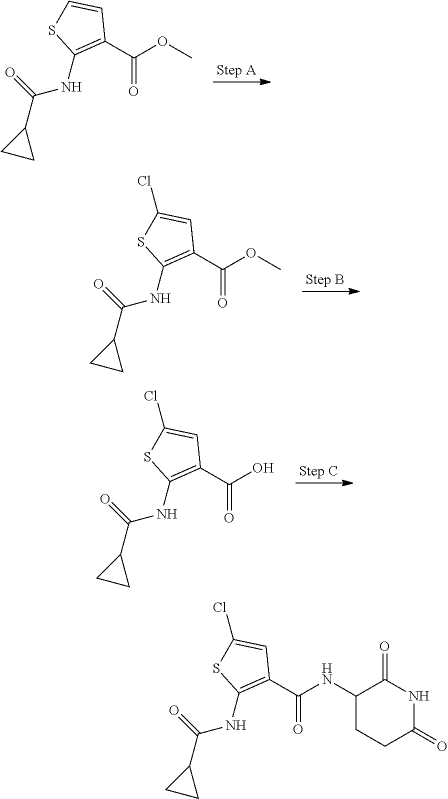

Step A: Methyl 5-chloro-2-cyclopropaneamidothiophene-3-carboxylate was synthesized using Example Method 1, above (80% yield) using methyl 2-cyclopropaneamidothiophene-3-carboxylate as a starting material.

Step B: 5-chloro-2-cyclopropaneamidothiophene-3-carboxylic acid was synthesized using Example Method 2, above (86% yield) using methyl 5-chloro-2-cyclopropaneamidothiophene-3-carboxylate as a starting material.

Step C: 5-chloro-2-cyclopropaneamido-N-(2,6-dioxopiperidin-3-yl)thiophene-3-carboxamide was synthesized using the general procedure shown in Reaction Scheme 1 and Synthetic Conditions B, above (30% yield), using 5-chloro-2-cyclopropaneamidothiophene-3-carboxylic acid as a starting material.

¹H NMR (500 MHz, DMSO) δ 12.11 (s, 1H), 10.88 (s, 1H), 8.69-8.60 (m, 1H), 7.50 (s, 1H), 4.79-4.69 (m, 1H), 2.84-2.72 (m, 1H), 2.61-2.53 (m, 1H), 2.21-2.08 (m, 1H), 2.04-1.92 (m, 2H), 0.98-0.84 (m, 4H).

LCMS (m/z [M+H]⁺): 356.2

### Example 7: Synthesis of N-(2,6-dioxopiperidin-3-yl)-3-acetamidothiophene-2-carboxamide (7)

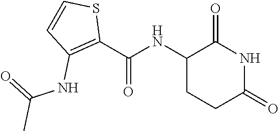 [SMILES: CC(=O)Nc1ccsc1C(=O)NC1CCC(=O)NC1=O]

This compound was synthesized using the general procedure shown in Reaction Scheme 1 and Synthetic Conditions B, above (15% yield), and N-(2,6-dioxopiperidin-3-yl)-3-acetamidothiophene-2-carboxamide as a starting material.

¹H NMR (400 MHz, DMSO) δ 10.89 (s, 2H), 8.55 (d, J=8.1 Hz, 1H), 7.94 (d, J=5.3 Hz, 1H), 7.75 (d, J=5.4 Hz, 1H), 4.76-4.66 (m, 1H), 2.85-2.69 (m, 1H), 2.59-2.52 (m, 1H), 2.25-2.12 (m, 1H), 2.09 (s, 3H), 2.01-1.89 (m, 1H).

LCMS (m/z [M+H]⁺): 295.8

### Example 8: Synthesis of N-(2,6-dioxopiperidin-3-yl)-2-acetamido-4-methoxythiophene-3-carboxamide (8)

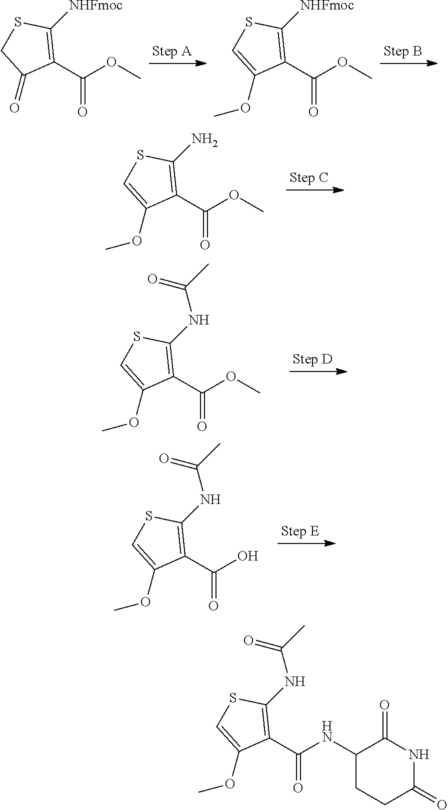

Step A: H₂SO₄ (1 mL) was added dropwise to a stirred suspension of methyl 2-({[(9H-fluoren-9-yl)methoxy]carbonyl}amino)-4-oxo-4,5-dihydrothiophene-3-carboxylate (9.65 g, 24.4 mmol) in MeOH (200 mL). The reaction mixture was refluxed for 16 h, cooled to RT and filtered to give 2-({[(9H-fluoren-9-yl)methoxy]carbonyl}amino)-4-methoxythiophene-3-carboxylate (63% yield).

Step B: Morpholine (13.5 g, 155 mmol) was added to a solution of methyl 2-({[(9H-fluoren-9-yl)methoxy]carbonyl}amino)-4-methoxythiophene-3-carboxylate (6.3 g, 15.4 mmol) in dichloromethane (100 mL) and the reaction mixture was stirred overnight at room temperature, concentrated under reduced pressure, diluted with MTBE, filtered, and rinsed with small amount of MTBE. The filtrate was evaporated in vacuo to give crude methyl 2-amino-4-methoxythiophene-3-carboxylate, which was used in the next step without further purification.

Step C: methyl 2-acetamido-4-methoxythiophene-3-carboxylate was obtained in 73% yield using Example Method 3, above, with methyl 2-amino-4-methoxythiophene-3-carboxylate as a starting material.

Step D: 2-acetamido-4-methoxythiophene-3-carboxylic acid was obtained in 20% yield using Example Method 2, above, with methyl 2-acetamido-4-methoxythiophene-3-carboxylate as a starting material.

Step E: N-(2,6-dioxopiperidin-3-yl)-2-acetamido-4-methoxythiophene-3-carboxamide was synthesized using the general procedure shown in Reaction Scheme 1 and Synthetic Conditions B, above (47% yield), and 2-acetamido-4-methoxythiophene-3-carboxylic acid as a starting material.

¹H NMR (500 MHz, DMSO) δ 12.05 (s, 1H), 10.92 (s, 1H), 8.30 (d, J=7.1 Hz, 1H), 6.14 (s, 1H), 4.76-4.66 (m, 1H), 3.83 (s, 3H), 2.82-2.70 (m, 1H), 2.58-2.52 (m, 1H), 2.19 (s, 3H), 2.16-2.06 (m, 2H)

LCMS (m/z [M+H]⁺): 326.2

### Example 9: Synthesis of 5-cyano-N-(2,6-dioxopiperidin-3-yl)-2-acetamidothiophene-3-carboxamide (9)

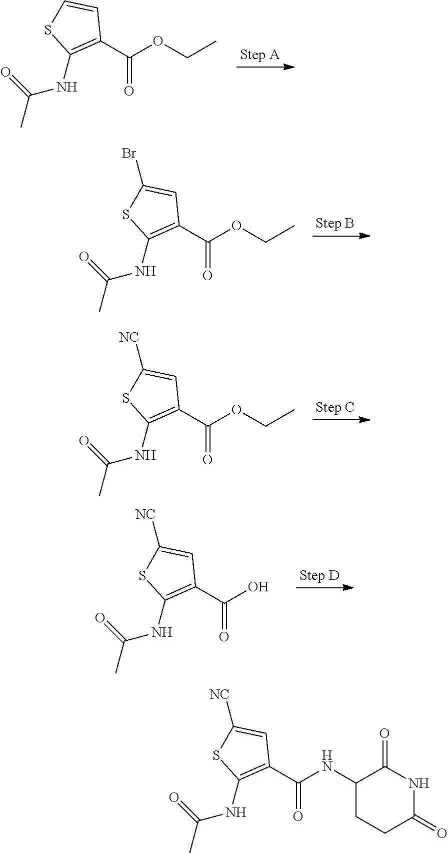

Step A: Ethyl 2-acetamidothiophene-3-carboxylate (11 g, 51.6 mmol) was dissolved in AcOH (110 mL) and solution of bromine (3.2 mL, 61.9 mmol) in AcOH (55 mL) was added dropwise over 15 min at RT. The reaction mixture was stirred at RT for 18 h, concentrated under reduced pressure and diluted water. The precipitate was filtered, washed with water and dried to give ethyl 5-bromo-2-acetamidothiophene-3-carboxylate (93% yield).

Step B: Zn(CN)₂ (8.45 g, 72 mmol) and Pd(dppf)Cl₂DCM (3.92 g, 4.8 mmol) were added to a solution of ethyl 5-bromo-2-acetamidothiophene-3-carboxylate (14 g, 48 mmol) in DMF (120 mL). Argon was bubbled through the reaction mixture for 10 min, then the reaction mixture was stirred at 150° C. for 16 h, cooled to RT, filtered and washed with EtOAc. The organic layer was dried over Na₂SO₄, concentrated under reduced pressure and purified by flash column chromatography to give 5-cyano-2-acetamidothiophene-3-carboxylate (83% yield).

Step C: Ethyl 5-cyano-2-acetamidothiophene-3-carboxylate (9.45 g, 39.7 mmol) was dissolved in EtOH:THF solution (120 mL:360 mL), the solution was cooled to +5° C. and lithium hydroxide monohydrate (11.7 g, 278 mmol) in H₂O (120 mL) was added dropwise over 20 min. The reaction mixture was stirred at RT for 18 h, concentrated under reduced pressure and acidified with 15% citric acid. The product was extracted with EtOAc, dried over Na₂SO₄ and evaporated under reduced pressure to give 5-cyano-2-acetamidothiophene-3-carboxylic acid (57% yield).

Step D: 5-cyano-N-(2,6-dioxopiperidin-3-yl)-2-acetamidothiophene-3-carboxamide was synthesized using the general procedure shown in Reaction Scheme 1 and Synthetic Conditions A, above (35% yield), and 5-cyano-2-acetamidothiophene-3-carboxylic acid as a starting material.

¹H NMR (400 MHz, DMSO) δ 12.08 (s, 1H), 10.94 (s, 1H), 8.89 (d, J=7.9 Hz, 1H), 8.27 (s, 1H), 4.82-4.65 (m, 1H), 2.87-2.72 (m, 1H), 2.62-2.53 (m, 1H), 2.30 (s, 3H), 2.24-2.08 (m, 1H), 2.06-1.93 (m, 1H)

LCMS (m/z [M+H]⁺): 321.0

### Example 10: Synthesis of 4-cyclopropyl-N-(2,6-dioxopiperidin-3-yl)-2-acetamidothiophene-3-carboxamide (10)

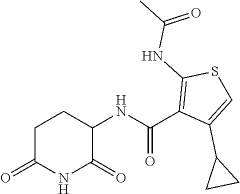 [SMILES: CC(=O)Nc1scc(C2CC2)c1C(=O)NC1CCC(=O)NC1=O]

This compound was synthesized using the general procedure shown in Reaction Scheme 1 and Synthetic Conditions B, above (23% yield), and 4-chloro-N-(2,6-dioxopiperidin-3-yl)-2-acetamidothiophene-3-carboxamide as a starting material.

¹H NMR: (400 MHz, DMSO-d₆) δ 11.21 (s, 1H), 11.05 (s, 1H), 8.30 (d, J=7.7 Hz, 1H), 6.57 (s, 1H), 4.94-4.79 (m, 1H), 2.88-2.74 (m, 1H), 2.63-2.52 (m, 1H), 2.16 (5, 3H), 2.13-2.04 (m, 2H), 2.03-1.95 (m, 2H), 0.98-0.83 (m, 2H), 0.72-0.64 (m, 1H), 0.64-0.55 (m, 1H)

LCMS (m/z [M+H]′): 336.3

### Example 11: Synthesis of 4,5-dichloro-N-(2,6-dioxopiperidin-3-yl)-2-acetamidothiophene-3-carboxamide (11)

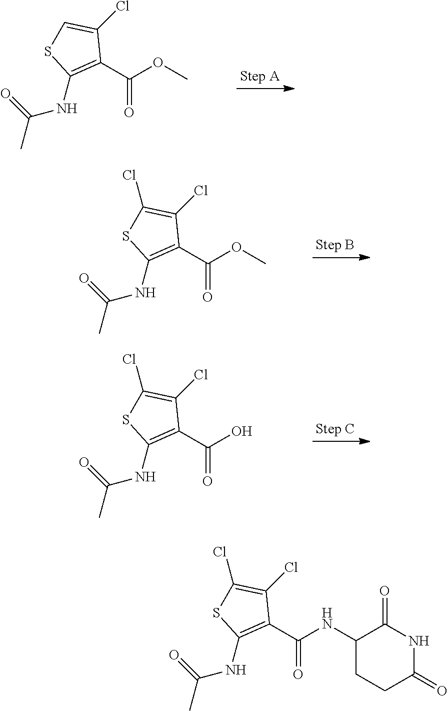

Step A: methyl 4,5-dichloro-2-acetamidothiophene-3-carboxylate was prepared in 90% yield using Example Method 1, above, with methyl 4-chloro-2-acetamidothiophene-3-carboxylate as a starting material.

Step B: 4,5-Dichloro-2-acetamidothiophene-3-carboxylic acid was prepared in 70% yield using Example Method 2, above, with methyl 4,5-chloro-2-acetamidothiophene-3-carboxylate as a starting material.

Step C: 4,5-dichloro-N-(2,6-dioxopiperidin-3-yl)-2-acetamidothiophene-3-carboxamide was synthesized in 40% yield using the general procedure shown in Reaction Scheme 1 and Synthetic Conditions A, above, and 4,5-dichloro-2-acetamidothiophene-3-carboxylic acid as a starting material.

¹H NMR: (500 MHz, DMSO) d 11.20 (s, 1H), 11.09 (s, 1H), 8.74-8.67 (m, 1H), 4.93-4.80 (m, 1H), 2.88-2.75 (m, 1H), 2.62-2.52 (m, 1H), 2.19 (s, 3H), 2.14-2.02 (m, 2H).

LCMS (m/z [M−H]⁻): 362.1

### Example 12: Synthesis of 5-Chloro-N-(2,6-dioxopiperidin-3-yl)-2-acetamidothiophene-3-carboxamide (12)

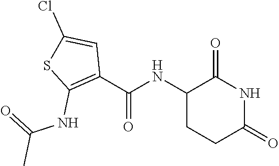 [SMILES: CC(=O)Nc1sc(Cl)cc1C(=O)NC1CCC(=O)NC1=O]

This compound was synthesized using the general procedure shown in Reaction Scheme 1 and Synthetic Conditions B above (22% yield), with 5-chloro-2-acetamidothiophene-3-carboxylic acid as a starting material.

¹H NMR (500 MHz, DMSO) δ 11.84 (s, 1H), 10.91 (s, 1H), 8.67 (d, J=8.0 Hz 1H), 7.50 (s, 1H), 4.77-4.65 (m, 1H), 2.84-2.73 (m, 1H), 2.61-2.54 (m, 1H), 2.22 (s, 3H), 2.19-2.07 (m, 1H), 2.02-1.91 (m, 1H)

LCMS (m/z [M+H]⁺): 329.8

### Example 13: Synthesis of N-(2,6-dioxopiperidin-3-yl)-2-methanesulfonamidothiophene-3-carboxamide (13)

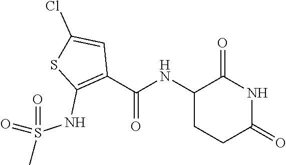 [SMILES: CS(=O)(=O)Nc1sc(Cl)cc1C(=O)NC1CCC(=O)NC1=O]

This compound was synthesized using the general procedure shown in Reaction Scheme 1 and Synthetic Conditions B, above (26% yield), with 2-(methylsulfonamido)thiophene-3-carboxylic acid as a starting material.

¹H NMR (400 MHz, DMSO) δ 11.12 (s, 1H), 10.92 (s, 1H), 8.80-8.65 (m, 1H), 7.46-7.36 (m, 1H), 7.17-7.06 (m, 1H), 4.82-4.69 (m, 1H), 3.17 (s, 3H), 2.85-2.72 (m, 1H), 2.60-2.53 (m, 1H), 2.20-2.04 (m, 1H), 2.02-1.92 (m, 1H)

LCMS (m/z [M+H]⁺): 332.2

### Example 14: Synthesis of 5-acetamido-N⁴-(2,6-dioxopiperidin-3-yl)-5-acetamido-N²-methylthiophene-2,4-dicarboxamide (14)

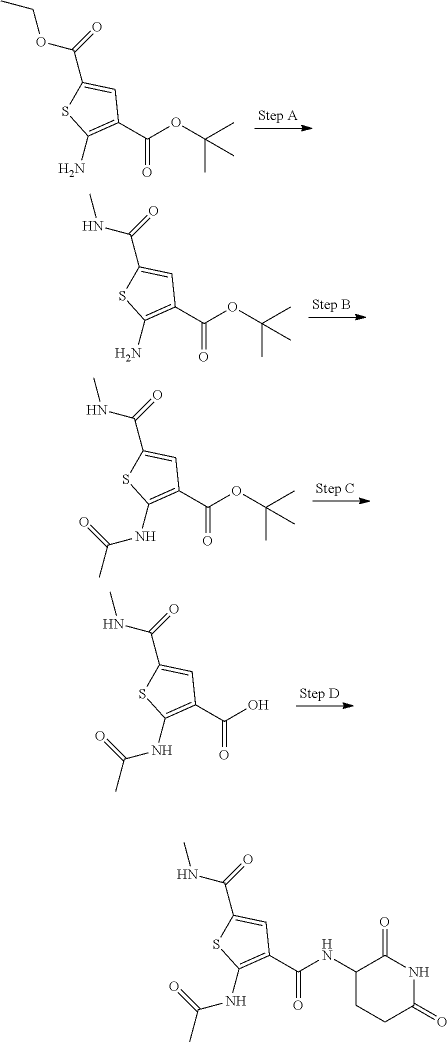

Step A: 4-tert-butyl 2-ethyl 5-aminothiophene-2,4-dicarboxylate (3.71 g, 13.7 mmol) was added to 20% solution of methylamine in methanol (20 mL) and the reaction mixture was stirred for 5 days at 70° C., concentrated under reduced pressure and triturated with isopropyl alcohol:hexane (1:1). The precipitate was filtered to give tert-butyl 2-amino-5-(methylcarbamoyl)thiophene-3-carboxylate (93% yield).

Step B: Triethylamine (3.3 g, 32.6 mmol), DMAP (0.13 g, 1.06 mmol) and acetic acid (1.67 g, 27.8 mmol) were added to a solution of tert-butyl 2-amino-5-(methylcarbamoyl)thiophene-3-carboxylate (2.8 g, 10.9 mmol) in dry MeCN (30 mL). The reaction mixture was stirred overnight at 50° C., cooled to room temperature, diluted with water, extracted with DCM, dried over Na₂SO₄ and concentrated under reduced pressure to give tert-butyl 2-acetamido-5-(methylcarbamoyl)thiophene-3-carboxylate (95% yield).

Step C: 10% HCl in dioxane (20 mL) was added to a solution of tert-butyl 2-acetamido-5-(methylcarbamoyl)thiophene-3-carboxylate (3.1 g, 10.4 mmol) in DCM (20 mL) and the reaction mixture was stirred for 3 days at RT. The precipitate was filtered, washed with DCM and dried to give 2-acetamido-5-(methylcarbamoyl)thiophene-3-carboxylic acid (60% yield).

Step D: N⁴-(2,6-dioxopiperidin-3-yl)-5-acetamido-N²-methylthiophene-2,4-dicarboxamide was synthesized using the general procedure shown in Reaction Scheme 1 and Synthetic Conditions B, above (44% yield), and 2-acetamido-5-(methylcarbamoyl)thiophene-3-carboxylic acid as a starting material.

¹H NMR (400 MHz, DMSO) δ 11.84 (s, 1H), 10.92 (s, 1H), 8.78 (d, J=8.1 Hz, 1H), 8.35-8.25 (m, 1H), 7.96 (s, 1H), 4.81-4.68 (m, 1H), 2.85-2.74 (m, 1H), 2.73 (d, J=4.4 Hz, 3H), 2.62-2.52 (m, 1H), 2.24 (s, 3H), 2.20-2.07 (m, 1H), 2.04-1.93 (m, 1H)

LCMS (m/z [M+H]⁺): 352.9

### Example 15: Synthesis of N-(2,6-dioxopiperidin-3-yl)-2-oxo-2,3-dihydrothiazole-4-carboxamide (15)

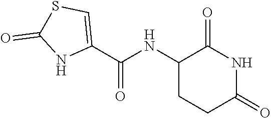 [SMILES: O=C1CCC(NC(=O)c2csc(=O)[nH]2)C(=O)N1]

This compound was synthesized using the general procedure shown in Reaction Scheme 1 and Synthetic Conditions C, above, (24% yield) using 2-oxo-2,3-dihydrothiazole-4-carboxylic acid (20 mg) as a starting material.

¹H NMR (500 MHz, DMSO) δ 11.62 (s, 1H), 10.89 (s, 1H), 8.69 (d, J=8.3 Hz, 1H), 7.17 (s, 1H), 4.68 (ddd, =12.5, 8.3, 5.4 Hz, 1H), 2.78 (ddd, =17.3, 13.2, 5.7 Hz, 1H), 2.60-2.52 (m, 1H), 2.11-2.00 (m, 1H), 1.96 (dtd, J=12.7, 5.5, 2.8 Hz, 1H).

LCMS (m/z [M+H]⁺): 256.2

### Example 16: Synthesis of 5-chloro-N-(2,6-dioxopiperidin-3-yl)-2-(methylamino)thiophene-3-carboxamide (16)

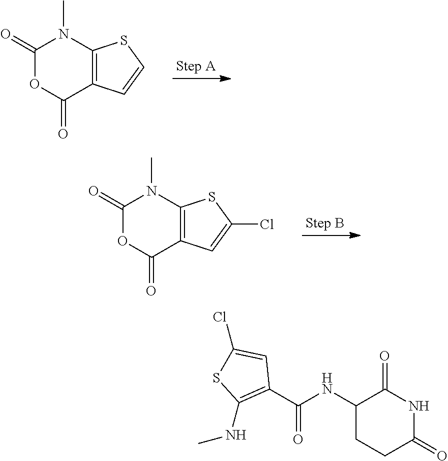 [SMILES: Cn1c(=O)oc(=O)c2ccsc21 ; Cn1c(=O)oc(=O)c2cc(Cl)sc21 ; CNc1sc(Cl)cc1C(=O)NC1CCC(=O)NC1=O]

Step A: N-chlorosuccinimide (0.884 g, 6.62 mmol) was added to a solution of 1-methyl-1H,2H,4H-thieno[2,3-d][1,3]oxazine-2,4-dione (1 g, 5.46 mmol) in mixture of toluene (4 mL) and acetic acid (4 mL). The reaction mixture was stirred at 70° C. for 2 h, concentrated under reduced pressure, diluted with water and filtered. The solids were washed with water and dried 6-chloro-1-methyl-1H,2H,4H-thieno[2,3-d][1,3]oxazine-2,4-dione (72% yield).

Step B: 3-Aminopiperidine-2,6-dione hydrochloride (0.655 g, 3.98 mmol) and triethylamine (0.483 g, 4.77 mmol) were added to a solution of 6-chloro-1-methyl-1H,2H,4H-thieno[2,3-d][1,3]oxazine-2,4-dione (0.865 g, 3.97 mmol) in ethanol (20 mL) and the reaction mixture was refluxed for 18 h, concentrated under reduced pressure and diluted with water. The precipitate was filtered, washed with water and isopropyl alcohol, and dried to give 5-chloro-N-(2,6-dioxopiperidin-3-yl)-2-(methylamino)thiophene-3-carboxamide (44% yield).

¹H NMR (400 MHz, DMSO) δ 10.79 (s, 1H), 8.18-8.04 (m, 1H), 8.03-7.91 (m, 1H), 7.26 (s, 1H), 4.68-4.52 (m, 1H), 2.85 (s, 3H), 2.79-2.67 (m, 1H), 2.60-2.53 (m, 1H), 2.16-2.01 (m, 1H), 1.99-1.85 (m, 1H).

LCMS (m/z [M+H]⁺): 302.2

### Example 17: Synthesis of 5-chloro-N-(2,6-dioxopiperidin-3-yl)-2-[(2S)-2-hydroxypropanamido]thiophene-3-carboxamide (17)

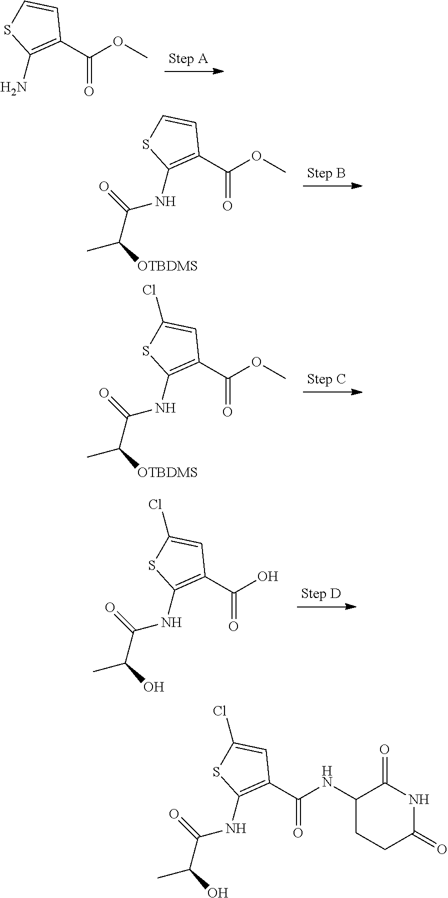

Step A: Oxalyl chloride (9.24 g, 72.8 mmol) and a drop of DMF were added to a solution of (2S)-2-[(tert-butyldimethylsilyl)oxy]propanoic acid (11.9 g, 58.2 mmol) in dry DCM (150 mL). The resulting mixture was stirred at RT for 2 h, concentrated under reduced pressure, dissolved in DCM (50 mL) and added dropwise to a cooled solution of methyl 2-aminothiophene-3-carboxylate (4.58 g, 29.1 mmol) and DIPEA (11.3 g, 87.4 mmol) in DCM (150 mL). The reaction mixture was stirred at RT for 2 h, washed with water, dried over Na₂SO₄, concentrated under reduced pressure and purified by flash column chromatography to give methyl 2-[(2S)-2-[(tert-butyldimethylsilyl)oxy]propanamido]thiophene-3-carboxylate (24% yield).

Step B: N-chlorosuccinimide (1.03 g, 7.71 mmol) was added to a solution of methyl 2-[(2.5)-2-[(tert-butyldimethylsilyl)oxy]propanamido]thiophene-3-carboxylate (2.4 g, 6.99 mmol) in DMF (30 mL) and the mixture was stirred at RT for 18 h. The reaction mixture was poured into water and extracted with EtOAc, dried over Na₂SO₄ and concentrated under reduced pressure to give methyl 2-[(2S)-2-[(tert-butyldimethylsilyl)oxy]propanamido]-5-chlorothiophene-3-carboxylate (91% yield).

Step C: 10% aqueous solution of LiOH (6 mL) was added to a solution of crude methyl 2-[(2S)-2-[(tert-butyldimethylsilyl)oxy]propanamido]-5-chlorothiophene-3-carboxylate (2.42 g, 6.40 mmol) in THF (12 mL) and the mixture was stirred at RT for 3 days. The reaction mixture was concentrated under reduced pressure, diluted with water and acidified with 10% HCl. The product was extracted into DCM, dried over Na₂SO₄, concentrated under reduced pressure and crystallized to give (S)-5-chloro-2-(2-hydroxypropanamido)thiophene-3-carboxylic acid (25% yield).

Step D: 3-Aminopiperidine-2,6-dione (0.421 g, 2.56 mmol, hydrochloride salt), 3H-[1,2,3]triazolo[4,5-b]pyridin-3-ol (0.191 g, 1.41 mmol), triethylamine (0.330 g, 3.27 mmol), and EDC (0.397 g, 2.56 mmol) were added sequentially to a solution of (S)-5-chloro-2-(2-hydroxypropanamido)thiophene-3-carboxylic acid (0.319 g, 1.28 mmol) in DMA (2.5 mL). The reaction mixture stirred at RT for 18 h, concentrated under reduced pressure and purified by HPLC to give 5-chloro-N-(2,6-dioxopiperidin-3-yl)-2-[(2S)-2-hydroxypropanamido]thiophene-3-carboxamide (29% yield).

¹H NMR (400 MHz, DMSO) δ 12.59 (s, 1H), 10.91 (s, 1H), 8.65 (d, J=8.3 Hz, 1H), 7.54 (s, 1H), 6.25 (d, J=4.8 Hz, 1H), 4.82-4.71 (m, 1H), 4.34-4.23 (m, 1H), 2.85-2.71 (m, 1H), 2.61-2.53 (m, 1H), 2.20-2.04 (m, 1H), 2.01-1.90 (m, 1H), 1.32 (d, 3H)

LCMS (m/z [M−1-1]+): 359.9

### Example 18: Synthesis of 5-chloro-N-(2,6-dioxopiperidin-3-yl)-3-acetamidothiophene-2-carboxamide (18)

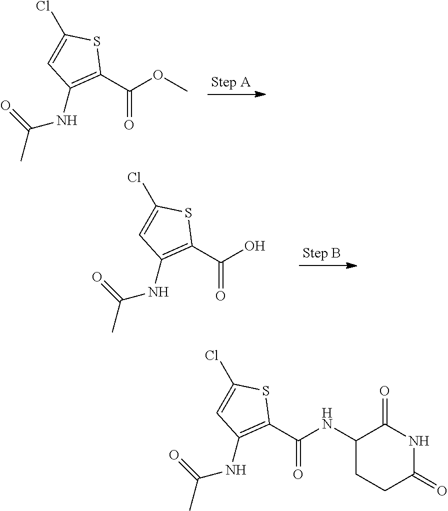 [SMILES: COC(=O)c1sc(Cl)cc1NC(C)=O ; CC(=O)Nc1cc(Cl)sc1C(=O)O ; CC(=O)Nc1cc(Cl)sc1C(=O)NC1CCC(=O)NC1=O]

Step A: 5-chloro-3-acetamidothiophene-2-carboxylic acid was obtained in 72% yield using Example Method 2, above, with methyl 5-chloro-3-acetamidothiophene-2-carboxylate as a starting material.

Step B: 5-chloro-N-(2,6-dioxopiperidin-3-yl)-3-acetamidothiophene-2-carboxamide was synthesized using the general procedure shown in Reaction Scheme 1 and Synthetic Conditions A, above (40% yield), and 5-chloro-3-acetamidothiophene-2-carboxylic acid as a starting material.

¹H NMR (400 MHz, DMSO) δ 10.91 (s, 1H), 10.87 (s, 1H), 8.61 (d, J=8.2 Hz, 1H), 7.90 (s, 1H), 4.75-4.64 (m, 1H), 2.85-2.70 (m, 1H), 2.60-2.52 (m, 2H), 2.11 (s, 3H), 2.00-1.91 (m, 1H)

LCMS (m/z [M+H]⁺): 330.2

### Example 19: Synthesis of 4-chloro-5-cyclopropyl-N-(2,6-dioxopiperidin-3-yl)-2-acetamidothiophene-3-carboxamide (19)

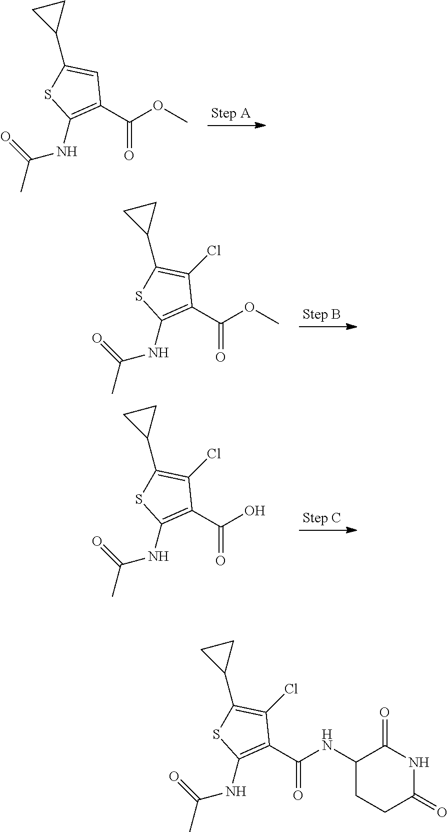

Step A: SO₂Cl₂ (0.207 g, 1.53 mmol) was added to a solution of methyl 5-cyclopropyl-2-acetamidothiophene-3-carboxylate (0.306 g, 1.28 mmol) in CHCl₃ (15 mL). The reaction mixture was refluxed for 2 h, concentrated under reduced pressure and diluted with water. The product was extracted with EtOAc, dried over Na₂SO₄ and concentrated under reduced pressure to give methyl 4-chloro-5-cyclopropyl-2-acetamidothiophene-3-carboxylate (81% yield).

Step B: 4-chloro-5-cyclopropyl-2-acetamidothiophene-3-carboxylic acid was obtained in 78% yield using Example Method 2, above, with methyl 4-chloro-5-cyclopropyl-2-acetamidothiophene-3-carboxylate as a starting material.

Step C: HATU (0.370 g, 0.973 mmol) was added to the solution of 4-chloro-5-cyclopropyl-2-acetamidothiophene-3-carboxylic acid (0.211 g, 0.812 mmol), 3-aminopiperidine-2,6-dione (0.134 g, 1.05 mmol) and N-methylmorpholine (0.205 g, 2.03 mmol) in DMF (5 mL) at 0° C. The reaction mixture was stirred overnight at room temperature, diluted with water, extracted with AcOEt, dried over Na₂SO₄, concentrated under reduced pressure and purified by HPLC to give 4-chloro-5-cyclopropyl-N-(2,6-dioxopiperidin-3-yl)-2-acetamidothiophene-3-carboxamide (41% yield).

¹H NMR (400 MHz, DMSO) δ 11.07 (s, 1H), 11.04 (s, 1H), 8.54 (d, J=8.2 Hz, 1H), 4.90-4.78 (m, 1H), 2.89-2.72 (m, 1H), 2.65-2.52 (m, 2H), 2.16 (s, 3H), 2.12-1.98 (m, 2H), 1.08-0.98 (m, 2H), 0.71-0.58 (m, 2H).

LCMS (m/z [M+H]⁺): 369.8

### Example 20: Synthesis of N-(2,6-dioxopiperidin-3-yl)-2-methoxythiophene-3-carboxamide (20)

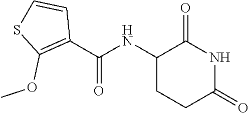 [SMILES: COc1sccc1C(=O)NC1CCC(=O)NC1=O]

This compound was synthesized using the general procedure shown in Reaction Scheme 1 and Synthetic Conditions B, above (17% yield), using 2-methoxythiophene-3-carboxylic acid as a starting material.

¹H NMR (400 MHz, DMSO) δ 10.86 (s, 1H), 7.81 (d, J=7.5 Hz, 1H), 7.10 (d, J=5.9 Hz, 1H), 6.82 (d, J=5.9 Hz, 1H), 4.74-4.63 (m, 1H), 4.09 (s, 3H), 2.83-2.69 (m, 1H), 2.57-2.51 (m, 1H), 2.18-1.99 (m, 2H)

LCMS (m/z [M+H]⁺): 269.0

### Example 21: Synthesis of (S)—N-(2,6-dioxopiperidin-3-yl)-2-methoxythiophene-3-carboxamide (21)

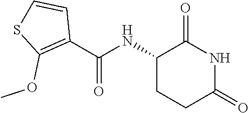 [SMILES: COc1sccc1C(=O)N[C@H]1CCC(=O)NC1=O]

This compound was synthesized using the general procedure shown in Reaction Scheme 1 and Synthetic Conditions D, above, (54% yield) using 2-methoxythiophene-3-carboxylic acid (20 mg) and (S)-3-aminopiperidine-2,6-dione as a starting material.

¹H NMR (500 MHz, DMSO) δ 10.86 (s, 1H), 7.81 (d, I=7.5 Hz, 1H), 7.11 (d, J=5.9 Hz, 1H), 6.83 (d, J=5.9 Hz, 1H), 4.76-4.61 (m, 1H), 4.05 (s, 3H), 2.77 (ddd, J=17.4, 13.3, 6.0 Hz, 1H), 2.48-2.44 (m, 1H), 2.17-2.01 (m, 2H).

LCMS (m/z [M+H]⁺): 268.9

### Example 22: Synthesis of (R)—N-(2,6-dioxopiperidin-3-yl)-2-methoxythiophene-3-carboxamide (22)

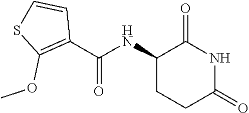 [SMILES: COc1sccc1C(=O)N[C@@H]1CCC(=O)NC1=O]

This compound was synthesized using the general procedure shown in Reaction Scheme 1 and Synthetic Conditions D, above, (49.5% yield) using 2-methoxythiophene-3-carboxylic acid (20 mg) and (R)-3-aminopiperidine-2,6-dione as a starting material.

¹H NMR (500 MHz, DMSO) δ 10.86 (s, 1H), 7.81 (d, J=7.5 Hz, 1H), 7.11 (d, J=5.9 Hz, 1H), 6.83 (d, J=5.9 Hz, 1H), 4.78-4.52 (m, 1H), 4.05 (s, 3H), 2.77 (ddd, J=17.4, 13.3, 6.0 Hz, 1H), 2.48-2.45 (m, 1H), 2.17-1.99 (m, 2H).

LCMS (m/z [M+H]⁺): 268.9

### Example 23: Synthesis of 2-methoxy-N-(1-methyl-2,6-dioxopiperidin-3-yl)thiophene-3-carboxamide (23)

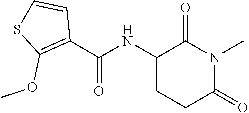

This compound was synthesized using the general procedure shown in Reaction Scheme 1 and Synthetic Conditions C, above, (61% yield) using 2-methoxythiophene-3-carboxylic acid (20 mg) and 3-amino-1-methylpiperidine-2,6-dione trifluoroacetic acid salt (1.2 eq.) as a starting material.

¹H NMR (500 MHz, DMSO) δ 7.86 (d, J=7.5 Hz, 1H), 7.11 (d, J=5.9 Hz, 1H), 6.82 (d, J=5.9 Hz, 1H), 4.76 (ddd, J=12.2, 7.4, 5.7 Hz, 1H), 4.05 (s, 3H), 3.01 (s, 3H), 2.85 (ddd, J=17.4, 13.1, 6.1 Hz, 1H), 2.67 (ddd, J=17.3, 4.5, 2.9 Hz, 1H), 2.17-2.01 (m, 2H).

LCMS (m/z [M+H]⁺): 283.1

### Example 24: Synthesis of methyl 2-(3-(2-methoxythiophene-3-carboxamido)-2,6-dioxopiperidin-1-yl)acetate (25)

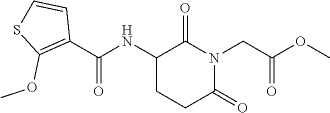

This compound was synthesized using the general procedure shown in Reaction Scheme 1 and Synthetic Conditions C, above, (44% yield) using 2-methoxythiophene-3-carboxylic acid (25.5 mg, 1.1 eq.) and methyl 2-(3-amino-2,6-dioxopiperidin-1-yl)acetate (trifluoroacetic acid salt, 1.0 eq.) as a starting materials.

¹H NMR (500 MHz, DMSO) δ 7.88 (d, J=7.6 Hz, 1H), 7.11 (d, J=6.0 Hz, 1H), 6.83 (d, J=5.9 Hz, 1H), 4.84 (ddd, J=12.8, 7.6, 5.4 Hz, 1H), 4.50-4.34 (m, 2H), 4.05 (s, 3H), 3.66 (s, 3H), 3.03-2.91 (m, 1H), 2.76 (ddd, J=17.6, 4.3, 2.5 Hz, 1H), 2.24-2.13 (m, 1H), 2.13-2.05 (m, 1H).

LCMS (m/z [M+H]⁺): 341.2

### Example 25: Synthesis of N-(2,6-dioxopiperidin-3-yl)-2-methoxythiophene-3-sulfonamide (27)

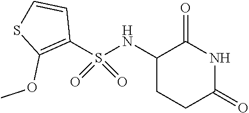 [SMILES: COc1sccc1S(=O)(=O)NC1CCC(=O)NC1=O]

To the cooled to 0° C. mixture of 3-aminopiperidine-2,6-dione hydrochloride (1.5 eq) and triethylamine (5 eq) in DCM (1.5 mL) was added 2-methoxythiophene-3-sulfonyl chloride (20 mg). Reaction was stirred for 18 h at RT, concentrated under reduced pressure and purified by flash column chromatography to give N-(2,6-dioxopiperidin-3-yl)-2-methoxythiophene-3-sulfonamide (66% yield).

¹H NMR (500 MHz, DMSO) δ 10.77 (s, 1H), 7.79 (d, J=8.0 Hz, 1H), 6.98 (d, J=6.0 Hz, 1H), 6.86 (d, J=6.0 Hz, 1H), 4.15 (dtd, J=11.9, 9.3, 8.7, 5.8 Hz, 1H), 3.99 (s, 3H), 2.68 (td, J=12.1, 6.1 Hz, 1H), 2.48-2.44 (m, 1H), 1.92-1.78 (m, 2H).

LCMS (m/z [M+H]⁺): 305.1

### Example 26: Synthesis of N-(2,6-dioxopiperidin-3-yl)-2-ethoxythiophene-3-carboxamide (28)

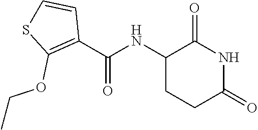 [SMILES: CCOc1sccc1C(=O)NC1CCC(=O)NC1=O]

This compound was synthesized using the general procedure shown in Reaction Scheme 1 and Synthetic Conditions D, above, (78% yield) using 2-ethoxythiophene-3-carboxylic acid (15 mg) as a starting material.

¹H NMR (500 MHz, DMSO) δ 10.90 (s, 1H), 7.86 (d, J=6.7 Hz, 1H), 7.09 (d, J=5.9 Hz, 1H), 6.83 (d, J=5.9 Hz, 1H), 4.67 (ddd, J=12.3, 6.7, 5.3 Hz, 1H), 4.28 (q, 1=7.0 Hz, 2H), 2.77 (ddd, J=16.8, 13.3, 5.2 Hz, 1H), 2.57-2.52 (m, 1H), 2.18 (dtd, J=12.9, 5.4, 2.4 Hz, 1H), 2.01 (qd, J=12.8, 4.5 Hz, 1H), 1.45 (t, I=7.0 Hz, 3H).

LCMS (m/z [M+H]⁺): 283.1;

### Example 27: Synthesis of N-(2,6-dioxopiperidin-3-yl)-2-methoxy-4-methylthiophene-3-carboxamide (29)

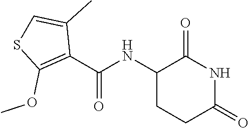 [SMILES: COc1scc(C)c1C(=O)NC1CCC(=O)NC1=O]

This compound was synthesized using the general procedure shown in Reaction Scheme 1 and Synthetic Conditions C, above, (21% yield) using 2-methoxy-4-methylthiophene-3-carboxylic acid (10 mg) as a starting material.

¹H NMR (500 MHz, DMSO) δ 10.83 (s, 1H), 7.97 (d, J=7.6 Hz, 1H), 6.47 (q, J=1.2 Hz, 1H), 4.66 (ddd, J=11.5, 7.6, 6.1 Hz, 1H), 3.98 (s, 3H), 2.75 (ddd, J=17.3, 12.5, 6.5 Hz, 1H), 2.52 (dd, J=8.0, 4.2 Hz, 1H), 2.23 (d, J=1.1 Hz, 3H), 2.12-1.99 (m, 2H).

LCMS (m/z [M−H]⁻): 281.1

### Example 28: Synthesis of N-(2,6-dioxopiperidin-3-yl)-2-methoxy-5-methylthiophene-3-carboxamide (30)

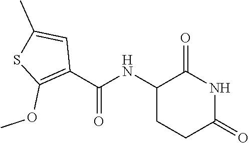 [SMILES: COc1sc(C)cc1C(=O)NC1CCC(=O)NC1=O]

This compound was synthesized using the general procedure shown in Reaction Scheme 1 and Synthetic Conditions C, above, (76% yield) using 2-methoxy-5-methylthiophene-3-carboxylic acid (20 mg) as a starting material.

¹H NMR (500 MHz, DMSO) δ 10.85 (s, 1H), 7.74 (d, J=7.5 Hz, 1H), 6.80 (q, J=1.2 Hz, 1H), 4.68 (ddd, J=11.9, 7.5, 6.0 Hz, 1H), 4.00 (s, 3H), 2.77 (ddd, J=17.3, 12.9, 6.3 Hz, 1H), 2.53 (q, J=1.5 Hz, 1H), 2.33 (d, J=1.3 Hz, 3H), 2.15-2.00 (m, 2H).

LCMS (m/z [M+H]⁺): 283.05

### Example 29: Synthesis of 5-(aminomethyl)-N-(2,6-dioxopiperidin-3-yl)-2-methoxythiophene-3-carboxamide (31)

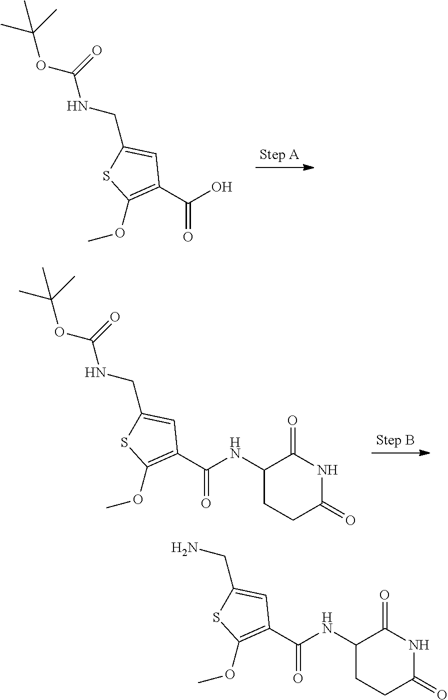

Step A: Tert-butyl N-({4-[(2,6-dioxopiperidin-3-yl)carbarmoyl]-5-methoxythiophen-2-yl}methyl)carbamate) was synthesized using the general procedure shown in Reaction Scheme 1 and Synthetic Conditions C, above (47% yield), and 5-(((tert-butoxycarbonyl)amino)methyl)-2-methoxythiophene-3-carboxylic acid (20 mg) as a starting material.

Step B: To a solution of tert-butyl N-({4-[(2,6-dioxopiperidin-3-yl)carbamoyl]-5-methoxythiophen-2-yl}methyl)carbamate) (8.7 mg, 0.022 mmol, 1 eq.) in dioxane (2 mL) was added 36% HCl (0.2 mL). The reaction was stirred at rt for 3 h and concentrated under reduced pressure to give a 5-(aminomethyl)-N-(2,6-dioxopiperidin-3-yl)-2-methoxythiophene-3-carboxamide hydrochloride (100% yield).

¹H NMR (500 MHz, DMSO) δ 10.86 (s, 1H), 8.15 (s, 3H), 7.78 (d, J=7.6 Hz, 1H), 7.28 (s, 1H), 4.69 (ddd, J=12.7, 7.6, 5.5 Hz, 1H), 4.06 (s, 3H), 3.39 (s, 2H), 2.77 (ddd, J=17.2, 13.5, 5.7 Hz, 1H), 2.59-2.52 (m, 1H), 2.11 (qd, J=12.9, 4.4 Hz, 1H), 2.03 (ddt, J=10.2, 5.7, 2.9 Hz, 1H).

LCMS (m/z [M+H]⁺): 298.1

### Example 30: Synthesis of 5-cyclopropyl-N-(2,6-dioxopiperidin-3-yl)-2-methoxythiophene-3-carboxamide (32)

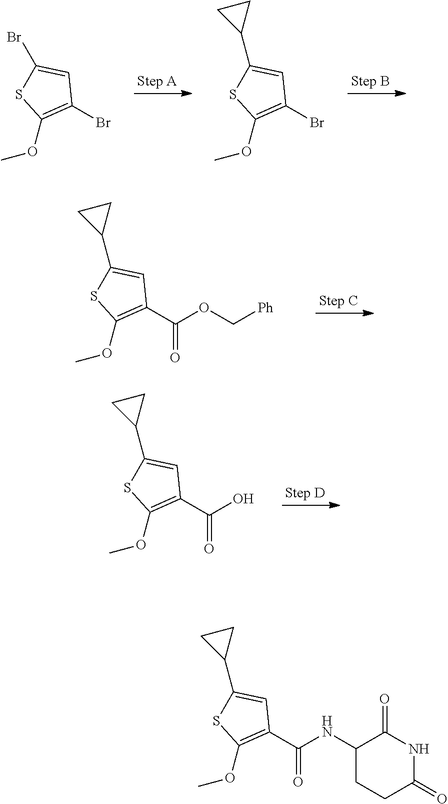

Step A: To a stirred solution of 3,5-dibromo-2-methoxythiophene (500.0 mg, 1.845 mmol) in toluene (9 mL) was added cyclopropyl boronic acid (206 mg, 2.399 mmol) and K₃PO₄ (784 mg, 3.69 mmol) in water (3 ml), the reaction mixture was purged with argon for 15 min and then Pd(PPh₃)₄ (320 mg, 0.277 mmol) was added. The reaction was stirred at 90° C. for 20 h, filtered through celite bed, concentrated under reduced pressure and purified by flash column chromatography to give 3-bromo-5-cyclopropyl-2-methoxythiophene (34% yield).

Step B: To a stirred solution of 3-bromo-5-cyclopropyl-2-methoxythiophene (700 mg, 3 mmol) in THF (20 mL) was added n-BuLi (1.8 M in THF) (3.4 mL, 6.005 mmol) dropwise at −78° C. Reaction mixture was stirred for 1 h at −78° C. and benzyl chloroformate (0.86 mL, 6 mmol) was added dropwise. The reaction was continued for 1 h, quenched with water, extracted with ethyl acetate and concentrated under reduced pressure. The product was purified by flash column chromatography to give benzyl 5-cyclopropyl-2-methoxythiophene-3-carboxylate (23% yield).

Step C: To a stirred solution of benzyl 5-cyclopropyl-2-methoxythiophene-3-carboxylate (350 mg, 1.215 mmol) in THF (6 mL) and methanol (6 mL) at 5-10° C. was added 50% aq. NaOH (12 ml). The reaction mixture was stirred at RT for 16 h and acidified with 6 M HCl. The solids were filtered, washed with pentane and dried to give 5-cyclopropyl-2-methoxythiophene-3-carboxylic acid (76% yield).

Step D: 5-cyclopropyl-N-(2,6-dioxopiperidin-3-yl)-2-methoxythiophene-3-carboxamide was synthesized using the general procedure shown in Reaction Scheme 1 and Synthetic Conditions C, above, (76% yield) using 5-cyclopropyl-2-methoxythiophene-3-carboxylic acid (20 mg) as a starting material.

¹H NMR (500 MHz, DMSO) δ 10.85 (s, 1H), 7.74 (d, J=7.5 Hz, 1H), 6.78 (d, J=1.0 Hz, 1H), 4.67 (ddd, J=12.1, 7.5, 5.8 Hz, 1H), 4.00 (s, 3H), 2.77 (ddd, J=17.3, 13.1, 6.1 Hz, 1H), 2.60-2.51 (m, 1H), 2.15-1.95 (m, 3H), 0.95-0.88 (m, 2H), 0.65-0.59 (m, 2H).

LCMS (m/z [M+H]⁺): 309.0

### Example 31: Synthesis of N-(2,6-dioxopiperidin-3-yl)-2-methoxy-5-phenylthiophene-3-carboxamide (33)

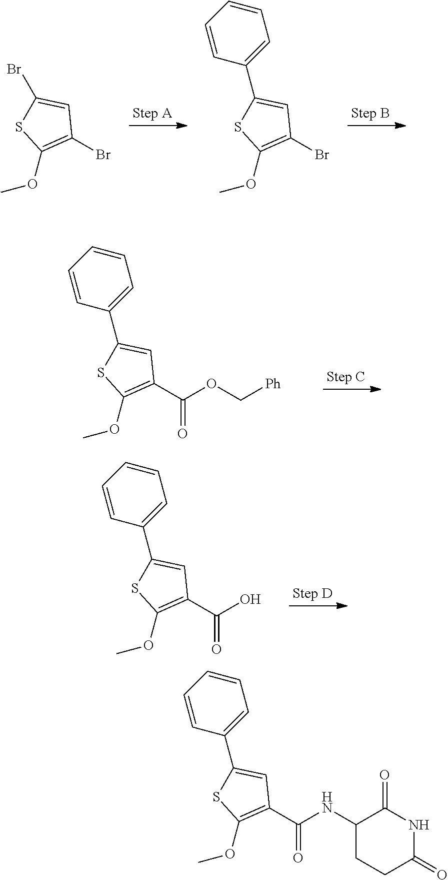

Step A: To 3,5-dibromo-2-methoxythiophene (4.0 g, 14.71 mmol) in dry THF (30 mL) was added 2.5M n-BuLi hexane solution (6.47 mL, 16.2 mmol) at −78° C. under argon atmosphere and the solution was stirred for 1 h. Tri-n-butyl borate (8.35 mL, 29.42 mmol) was added to the reaction mixture, the mixture was stirred for 1.5 h and warmed to RT. 20% Na₂CO₃ (33.6 mL), iodobenzene (1.65 mL, 14.71 mmol), and Pd(PPh₃)₄ (0.85 g, 0.73 mmol) were added and the reaction mixture was refluxed for 16 h. The reaction mixture was extracted with ether, dried over MgSO₄, concentrated under reduced pressure and purified by flash column chromatography to give 3-bromo-2-methoxy-5-phenylthiophene (50% yield).

Step B: 3-Bromo-2-methoxy-5-phenylthiophene (900 mg, 3.34 mmol) was dissolved in THF (15 mL) and cooled to −78° C. 1.8M n-BuLi in hexane (3.7 mL, 6.68 mmol) was added dropwise at −78° C. Reaction mixture was stirred for 1 h at −78° C. and benzyl chloroformate (0.95 mL, 6.68 mmol) was added dropwise. The reaction was continued for 1 h, quenched with water, extracted with ethyl acetate and concentrated under reduced pressure. The product was purified by flash column chromatography to give benzyl 2-methoxy-5-phenylthiophene-3-carboxylate (23% yield).

Step C: Benzyl 2-methoxy-5-phenylthiophene-3-carboxylate (230 mg, 0.71 mmol) was dissolved in THF (5 mL). MeOH (5 mL) and 50% NaOH solution (10 mL) were added and the reaction mixture was stirred at RT for 16 h and acidified with 6 M HCl. The solids were filtered, washed with pentane and dried to give 2-methoxy-5-phenylthiophene-3-carboxylic acid (130 mg, 78%) as off white solid.

Step D: N-(2,6-dioxopiperidin-3-yl)-2-methoxy-5-phenylthiophene-3-carboxamide was synthesized using the general procedure shown in Reaction Scheme 1 and Synthetic Conditions C, above, (71% yield) using 2-methoxy-5-phenylthiophene-3-carboxylic acid (20 mg) as a starting material.

¹H NMR (500 MHz, DMSO) δ 10.88 (s, 1H), 7.87 (d, J=7.6 Hz, 1H), 7.63-7.57 (m, 2H), 7.51 (s, 1H), 7.48-7.38 (m, 2H), 7.33-7.27 (m, 1H), 4.73 (ddd, J=12.7, 7.6, 5.6 Hz, 1H), 4.12 (s, 3H), 2.79 (ddd, J=17.3, 13.4, 5.8 Hz, 1H), 2.57-2.52 (m, 1H), 2.19-2.03 (m, 2H).

LCMS (m/z [M+H]⁺): 345.2

### Example 32: Synthesis of 5-bromo-N-(2,6-dioxopiperidin-3-yl)-2methoxythiophene-3-carboxamide (34)

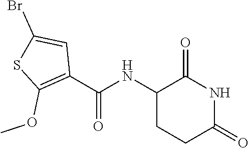 [SMILES: COc1sc(Br)cc1C(=O)NC1CCC(=O)NC1=O]

This compound was synthesized using the general procedure shown in Reaction Scheme 1 and Synthetic Conditions D, above, (21% yield) using 5-bromo-2-methoxythiophene-3-carboxylic acid (20 mg) as a starting material.

¹H NMR (500 MHz, DMSO) δ 10.85 (s, 1H), 7.82 (d, J=7.7 Hz, 1H), 7.19 (s, 1H), 4.77-4.61 (m, 1H), 4.04 (s, 3H), 2.76 (ddd, J=17.4, 13.5, 5.7 Hz, 1H), 2.56 (d, J=18.3 Hz, 1H), 2.14-2.04 (m, 1H), 2.01 (dtd, J=8.2, 5.6, 2.6 Hz, 1H).

LCMS (m/z [M+H]⁺): 346.8

### Example 33: Synthesis of 5-(tert-butyl)-N-(2,6-dioxopiperidin-3-O-2-methoxythiophene-3-carboxamide (35)

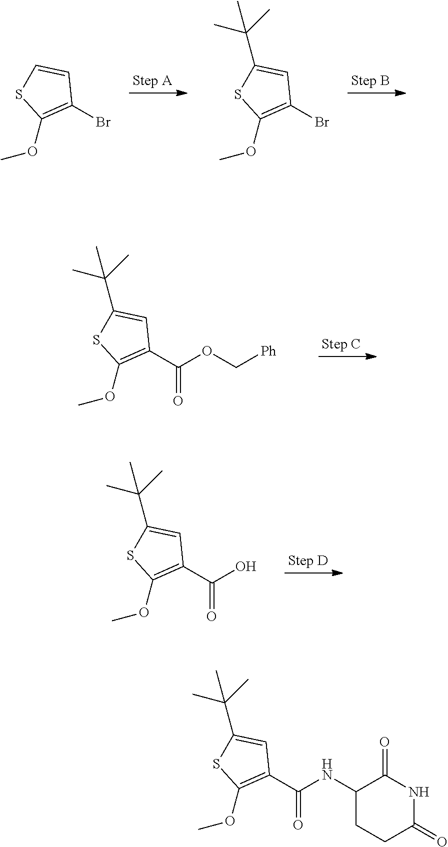

Step A: To stirred solution of AlCl₃ (2.1 g, 15.544 mmol) in DCM (20 mL) at −78° C. was added tert-butyl bromide (1.9 g, 13.472 mmol) in DCM (10 mL) dropwise at −78° C. and stirred for 20 min. 3-bromo-2-methoxythiophene (2 g, 10.363 mmol) in DCM (10 mL) was added dropwise stirred for 2 h. The reaction mixture was warmed to RT and stirred for another 16 h. The reaction mixture was quenched with water and extracted with DCM, concentrated under reduced pressure and purified by flash column chromatography to give 3-bromo-5-(tert-butyl)-2-methoxythiophene (31% yield).

Step B: To a stirred solution of 3-bromo-5-(tert-butyl)-2-methoxythiophene (900 mg, 3.614 mmol) in THF (22 mL) was added n-BuLi (1.8 M in THF) (4 ml, 7.229 mmol) dropwise at −78° C. Reaction mixture was stirred for 1 h at −78° C. and benzyl chloroformate (1.03 ml, 7.229 mmol) was added dropwise. The reaction was continued for 1 h, quenched with water, extracted with ethyl acetate and concentrated under reduced pressure. The product was purified by flash column chromatography to give benzyl 5-(tert-butyl)-2-methoxythiophene-3-carboxylate (220 mg, 20% yield) as light yellow oil.

Step C: To a stirred solution of benzyl 5-(tert-butyl)-2-methoxythiophene-3-carboxylate (450 mg, 1.47 mmol) in THF (8 mL) and methanol (8 mL) at 5° C. was added 50% aq. NaOH (16 mL). The reaction mixture was stirred at RT for 16 h and acidified with 6 M HCl. The solids were filtered, washed with pentane and dried to give 5-(tert-butyl)-2-methoxythiophene-3-carboxylic acid (69% yield).

Step D: 5-(tert-butyl)-N-(2,6-dioxopiperidin-3-yl)-2-methoxythiophene-3-carboxamide was synthesized using the general procedure shown in Reaction Scheme 1 and Synthetic Conditions C, above, (75% yield) using 5-(tert-butyl)-2-methoxythiophene-3-carboxylic acid (20 mg) as a starting material.

¹H NMR (500 MHz, DMSO) δ 10.85 (s, 1H), 7.75 (d, J=7.5 Hz, 1H), 6.83 (s, 1H), 4.68 (ddd, J=12.2, 7.5, 5.7 Hz, 1H), 4.02 (s, 3H), 2.77 (ddd, J=17.3, 13.3, 6.0 Hz, 1H), 2.57-2.52 (m, 1H), 2.17-1.99 (m, 2H), 1.31 (s, 9H).

LCMS (m/z [M+H]⁺): 325.2

### Example 34: Synthesis of 2-amino-N-(2,6-dioxopiperidin-3-yl)thiophene-3-carboxamide (36)

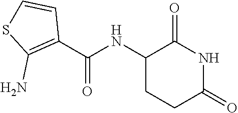 [SMILES: Nc1sccc1C(=O)NC1CCC(=O)NC1=O]

Tert-butyl (3-((2,6-dioxopiperidin-3-yl)carbamoyl)thiophen-2-yl)carbamate (1.0 g, 2.8 mmol) was dissolved in dichloromethane (10 mL) and a 10% solution of HCl in dioxane (3 mL) was added dropwise. The reaction mixture was stirred for 48 h at room temperature. The mixture was concentrated under reduced pressure and purified by preparative HPLC to give 2-amino-N-(2,6-dioxopiperidin-3-yl)thiophene-3-carboxamide (4% yield).

¹H NMR (500 MHz, DMSO) δ 10.78 (s, 1H), 7.96 (d, J=8.3 Hz, 1H), 7.22 (s, 2H), 7.07 (d, J=5.8 Hz, 1H), 6.28 (d, J=8.3 Hz, 1H), 4.69-4.61 (m, 1H), 2.82-2.68 (m, 1H), 2.57-2.52 (m, 1H), 2.15-2.04 (m, 1H), 1.96-1.84 (m, 1H).

LCMS (m/z [M+H]⁺): 254.0

### Example 35: Synthesis of 5-chloro-N-(2,6-dioxopiperidin-3-yl)-2-methoxythiophene-3-carboxamide (37)

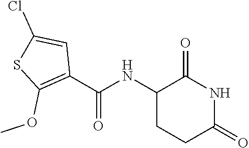 [SMILES: COc1sc(Cl)cc1C(=O)NC1CCC(=O)NC1=O]

This compound was synthesized using the general procedure shown in Reaction Scheme 1 and Synthetic Conditions A, above (24% yield), with 5-chloro-2-methoxythiophene-3-carboxylic acid as a starting material.

¹H NMR (400 MHz, DMSO) δ 10.87 (s, 1H), 7.83 (d, J=7.6 Hz, 1H), 7.08 (s, 1H), 4.74-4.60 (m, 1H), 4.04 (s, 3H), 2.85-2.68 (m, 1H), 2.58-2.51 (m, 1H), 2.18-1.93 (m, 2H)

LCMS (m/z [M+H]⁺): 303.0

### Example 36: Synthesis of N-{5-chloro-3-[(2,6-dioxopiperidin-3-yl)sulfamoyl]thiophen-2-yl}acetamide (38)

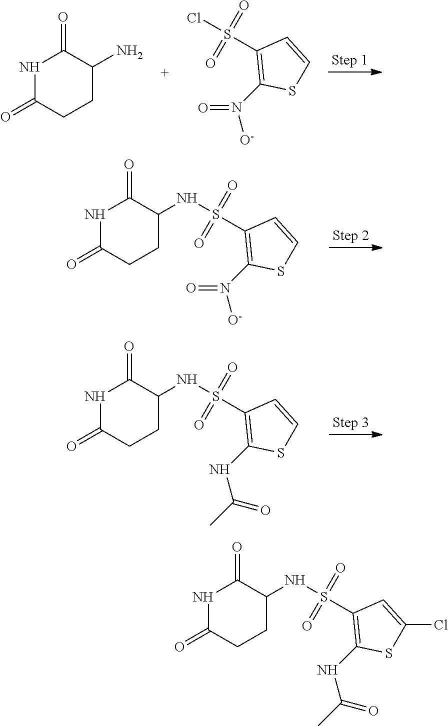

### Step 1: Synthesis of N-(2,6-dioxopiperidin-3-yl)-2-nitrothiophene-3-sulfonamide

2-Nitrothiophene-3-sulfonyl chloride (3.4 g, 15 mmol) was added to a solution of 3-aminopiperidine-2,6-dione (2.06 g, 12.5 mmol, hydrochloride salt) in pyridine (20 mL) cooled to −10° C. The resulting mixture was stirred for 16 h at room temperature. The mixture was concentrated under reduced pressure, diluted with water, acidified to pH=3, and extracted with EtOAc (3×20 mL). The combined organic layers were washed with water, dried over Na₂SO₄, and concentrated under reduced pressure. The crude product was purified by preparative HPLC to give 0.895 g of N-(2,6-dioxopiperidin-3-yl)-2-nitrothiophene-3-sulfonamide (22% yield).

### Step 2: Synthesis of N-{3-[(2,6-dioxopiperidin-3-yl)sulfamoyl]thiophen-2-yl}acetamide

Fe (0.47 g, 8.42 mmol) was added to a solution of N-(2,6-dioxopiperidin-3-yl)-2-nitrothiophene-3-sulfonamide (0.895 g, 2.80 mmol) in acetic acid (6 mL). The reaction mixture was stirred for 3 h at 50° C. Acetic anhydride (0.315 g, 3.09 mmol) was added under room temperature and the resulting mixture was stirred for 16 h at 50° C. The mixture was concentrated under reduced pressure and the residue was purified by preparative HPLC to give 0.362 g of N-{3-[(2,6-dioxopiperidin-3-yl)sulfamoyl]thiophen-2-yl}acetamide (45% yield).

### Step 3: Synthesis of N-{5-chloro-3-[(2,6-dioxopiperidin-3-yl)sulfamoyl]thiophen-2-yl}acetamide

NCS (0.161 g, 1.21 mmol) was added to a solution of N-{3-[(2,6-dioxopiperidin-3-yl)sulfamoyl]thiophen-2-yl}acetamide (0.362 g, 1.09 mmol) in DMF (2 mL). The reaction mixture was stirred for 16 h at room temperature, then it was diluted with water and extracted with EtOAc (3×10 mL). The organic extracts were dried over Na₂SO₄, concentrated, and purified by preparative HPLC to give 0.146 g of N-{5-chloro-3-[(2,6-dioxopiperidin-3-yl)sulfamoyl]thiophen-2-yl}acetamide (37% yields).

¹H NMR: (400 MHz, DMSO-d₅) δ 10.90 (s, 1H), 10.33 (s, 1H), 8.37 (d, J=8.0 Hz, 1H), 7.10 (s, 1H), 4.41-4.24 (m, 1H), 2.75-2.63 (m, 1H), 2.59-2.52 (m, 1H), 2.26 (s, 3H), 2.00-1.86 (m, 2H)

LCMS (m/z [M+H]⁻): 365.8

### Example 37: Synthesis of N-(2,6-dioxopiperidin-3-yl)-2-(1H-pyrazol-1-yl)thiophene-3-carboxamide (39)

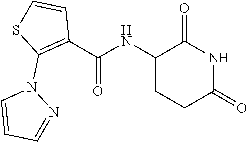 [SMILES: O=C1CCC(NC(=O)c2ccsc2-n2cccn2)C(=O)N1]

This compound was synthesized using the general procedure shown in Reaction Scheme 1 and Synthetic Conditions B, above (32% yield), and 2-(1H-pyrazol-1-yl)thiophene-3-carboxylic acid as a starting material.

¹H NMR (500 MHz, DMSO) δ 10.87 (s, 1H), 8.80 (d, J=8.3 Hz, 1H), 8.39-8.36 (m, 1H), 7.75-7.70 (m, 1H), 7.41 (d, J=5.5 Hz, 1H), 7.19 (d, J=5.5 Hz, 1H) 6.52-6.46 (m, 1H), 4.73-4.64 (m, 1H), 2.83-2.71 (m, 1H), 2.57-2.51 (m, 1H), 2.13-2.02 (m, 1H), 2.01-1.93 (m, 1H).

LCMS (m/z [M+H]⁺): 305.1

### Example 38: Synthesis of 2-amino-5-chloro-N-(2,6-dioxopiperidin-3-yl)thiophene-3-carboxamide (40)

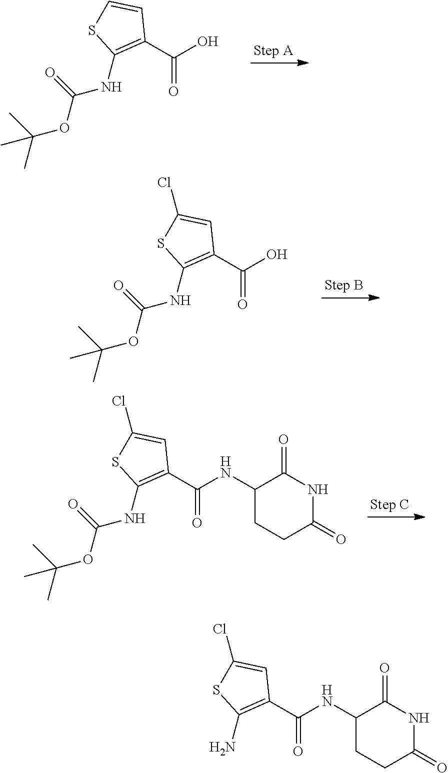

Step A: N-chlorosuccinimide (2.2 g, 16.5 mmol) was added to a solution of 2-((tert-butoxycarbonyl)amino)thiophene-3-carboxylic acid (3.3 g, 13.6 mmol) in DMF (20 mL) and the reaction mixture was stirred at RT for 2 h. The mixture was diluted with water and filtered. The solids were washed with water and dried to give 2-{[(tert-butoxy)carbonyl]amino}-5-chlorothiophene-3-carboxylic acid (84% yield).

Step B: Tert-butyl N-{5-chloro-3-[(2,6-dioxopiperidin-3-yl)carbamoyl]thiophen-2-yl}carbamate was synthesized using the general procedure shown in Reaction Scheme 1 and Synthetic Conditions A, above (81% yield), and 2-{[(tert-butoxy)carbonyl]amino}-5-chlorothiophene-3-carboxylic acid as a starting material.

Step C: 10% HCl in dioxane (2 mL) was added dropwise to a solution of the tert-butyl N-{5-chloro-3-[(2,6-dioxopiperidin-3-yl)carbamoyl]thiophen-2-yl}carbamate (2.0 g, 5.16 mmol) in dichloromethane (15 mL) and the mixture was stirred in ultrasonic bath for 8 h, concentrated under reduced pressure and purified by HPLC to give 2-amino-5-chloro-N-(2,6-dioxopiperidin-3-yl)thiophene-3-carboxamide (15% yield).

¹H NMR (400 MHz, DMSO) δ 10.81 (s, 1H), 7.97 (d, J=8.3 Hz, 1H), 7.41 (brs, 2H), 7.13 (s, 1H), 4.66-4.56 (m, 1H), 2.83-2.68 (m, 1H), 2.59-2.52 (m, 1H), 2.13-1.99 (m, 1H), 1.97-1.82 (m, 1H)

LCMS (m/z [M+H]⁺): 288.1

### Example 39: Synthesis of N-(2,6-dioxopiperidin-3-yl)-2-(1H-pyrrol-1-yl)thiophene-3-carboxamide (41)

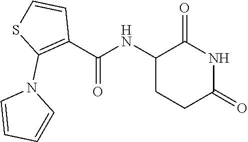 [SMILES: O=C1CCC(NC(=O)c2ccsc2-n2cccc2)C(=O)N1]

This compound was synthesized using the general procedure shown in Reaction Scheme 1 and Synthetic Conditions B, above (55% yield), and 2-(1H-pyrrol-1-yl)thiophene-3-carboxylic acid as a starting material.

¹H NMR (400 MHz, DMSO) δ 10.84 (s, 1H), 8.47 (d, J=8.3 Hz, 1H), 7.40 (d, J=5.7 Hz, 1H), 7.19 (d, J=5.7 Hz, 1H) 7.11-7.01 (m, 2H), 6.28-6.12 (m, 2H), 4.70-4.59 (m, 1H), 2.84-2.65 (m, 1H), 2.58-2.52 (m, 1H), 2.09-1.85 (m, 2H).

LCMS (m/z [M+H]⁺): 304.1

### Example 40: Synthesis of N-(2,6-dioxopiperidin-3-yl)-2-acetamido-5-(trifluoromethyl)thiophene-3-carboxamide (42)

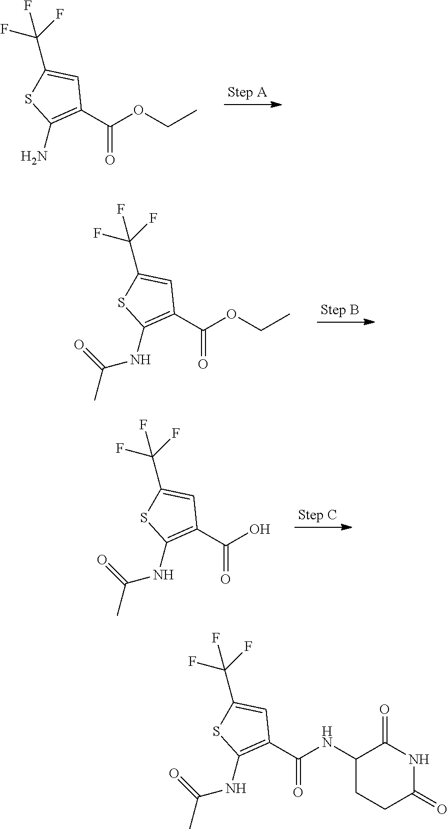 [SMILES: CCOC(=O)c1cc(C(F)(F)F)sc1N ; CCOC(=O)c1cc(C(F)(F)F)sc1NC(C)=O ; CC(=O)Nc1sc(C(F)(F)F)cc1C(=O)O ; CC(=O)Nc1sc(C(F)(F)F)cc1C(=O)NC1CCC(=O)NC1=O]

Step A: Triethylamine (0.397 g, 3.92 mmol) and acetic anhydride (0.400 g, 3.92 mmol) were added to a solution of ethyl 2-amino-5-(trifluoromethyl)thiophene-3-carboxylate (0.852 g, 3.56 mmol) in MeCN (15 mL). The reaction mixture was stirred overnight at 50° C., cooled to rt, concentrated under reduced pressure, and extracted with DCM, dried over Na₂SO₄, and concentrated to give ethyl 2-acetamido-5-(trifluoromethyl)thiophene-3-carboxylate (91% yield).

Step B: 10% solution of LiOH (0.081 g, 3.4 mmol) was added to a solution of ethyl 2-acetamido-5-(trifluoromethyl)thiophene-3-carboxylate (0.911 g, 3.24 mmol) in THF (15 mL) and the resulting mixture was stirred for 5 days at RT. The solvents were evaporated under reduced pressure, the residue was diluted with water and washed with MTBE. The aqueous layer acidified by citric acid and the precipitate was filtered, washed with water, and dried to give 2-acetamido-5-(trifluoromethyl)thiophene-3-carboxylic acid (28% yield).

Step C: N-(2,6-dioxopiperidin-3-yl)-2-acetamido-5-(trifluoromethyl)thiophene-3-carboxamide was synthesized using the general procedure shown in Reaction Scheme 1 and Synthetic Conditions A, above (26% yield), and 2-acetamido-5-(trifluoromethyl)thiophene-3-carboxylic acid as a starting material.

¹H NMR (400 MHz, DMSO) δ 11.98 (s, 1H), 10.93 (s, 1H), 9.00-8.81 (m, 1H), 8.13 (s, 1H), 4.87-4.62 (m, 1H), 2.98-2.65 (m, 2H), 2.28 (s, 3H), 2.20-2.08 (m, 1H), 2.07-1.88 (m, 1H).

LCMS (m/z [M+H]⁺): 364.2

### Example 41: Synthesis of 4-chloro-N-(2,6-dioxopiperidin-3-yl)-2-acetamidothiophene-3-carboxamide (43)

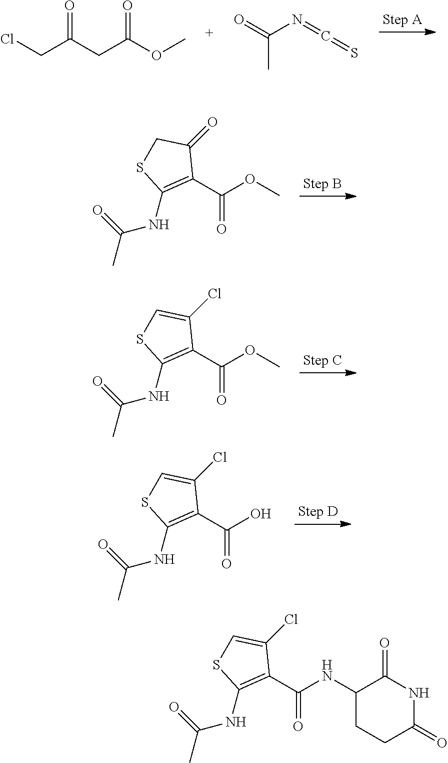

Step A: A solution of methyl 4-chloroacetoacetate (13.3 g, 88.3 mmol) in THF (30 mL) was added dropwise to a suspension of 60% NaH (4.45 g, 111 mmol) in THF (150 mL) at 0° C. After addition was completed, the reaction mixture was warmed to RT and stirred for 20 min. Then, the reaction mixture was cooled to 0° C. and a solution of acetyl isothiocyanate (8.92 g, 88.2 mmol) in THF (30 mL) was added dropwise. After the addition was completed, the reaction mixture was quenched at 0° C. by NH₄Cl solution, concentrated under reduced pressure and diluted with water and ethyl acetate. The mixture was filtered and the solids were dried to give methyl 2-acetamido-4-oxo-4,5-dihydrothiophene-3-carboxylate (4% yield).

Step B: POCl₃ (1.05 g, 6.85 mmol) was added to a suspension of methyl 2-acetamido-4-oxo-4,5-dihydrothiophene-3-carboxylate (0.74 g, 3.44 mmol) in dioxane (10 mL) and refluxed for 2 h. The reaction mixture was cooled and poured into iced water, the product was extracted with EtOAc, dried over Na₂SO₄ and concentrated under reduced pressure to give methyl 4-chloro-2-acetamidothiophene-3-carboxylate (26% yield).

Step C: 4-chloro-2-acetamidothiophene-3-carboxylic acid was synthesized using Example Method 2, above, with methyl 4-chloro-2-acetamidothiophene-3-carboxylate as a starting material.

Step D: 4-chloro-N-(2,6-dioxopiperidin-3-yl)-2-acetamidothiophene-3-carboxamide was synthesized using the general procedure shown in Reaction Scheme 1 and Synthetic Conditions B, above (17% yield), and 4-chloro-2-acetamidothiophene-3-carboxylic acid as a starting material.

¹H NMR (400 MHz, DMSO) δ 11.13 (s, 1H), 11.07 (s, 1H), 8.54 (d, J=8.2 Hz 1H), 7.09 (s, 1H), 4.93-4.79 (m, 1H), 2.89-2.74 (m, 1H), 2.62-2.52 (m, 1H), 2.19 (s, 3H), 2.15-2.01 (m, 2H).

LCMS (m/z [M−1-1]+): 330.0

### Example 42: Synthesis of 5-cyclopropyl-N-(2,6-dioxopiperidin-3-yl)-2-acetamidothiophene-3-carboxamide (44)

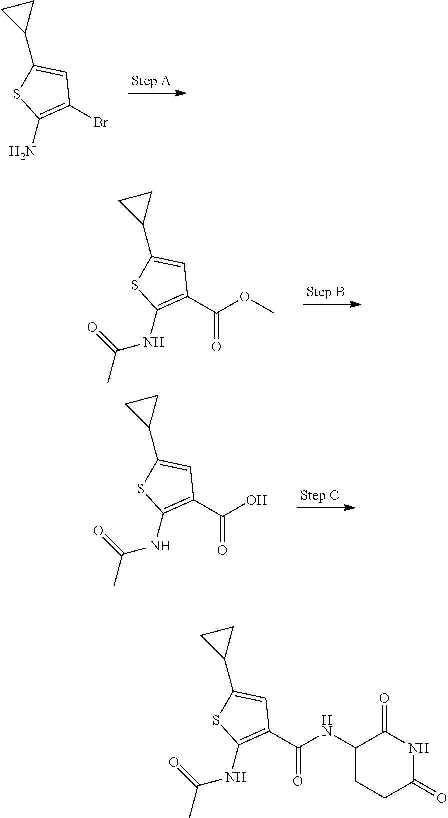

Step A: methyl 5-cyclopropyl-2-acetamidothiophene-3-carboxylate was synthesized in 69% yield using Example Method 3, above, using 2-amino-5-cyclopropylthiophene-3-carboxylate as a starting material.

Step B: 5-cyclopropyl-2-acetamidothiophene-3-carboxylic acid was synthesized in 57% yield using Example Method 2, above, and methyl 5-cyclopropyl-2-acetamidothiophene-3-carboxylate as a starting material.

Step C: 5-cyclopropyl-N-(2,6-dioxopiperidin-3-yl)-2-acetamidothiophene-3-carboxamide was synthesized using the general procedure shown in Reaction Scheme 1 and Synthetic Conditions B, above (56% yield), and 5-cyclopropyl-2-acetamidothiophene-3-carboxylic acid as a starting material.

¹H NMR (400 MHz, DMSO) δ 11.73 (s, 1H), 10.89 (s, 1H), 8.52 (d, J=8.5 Hz, 1H), 7.09 (s, 1H), 4.76-4.64 (m, 1H), 2.85-2.71 (m, 1H), 2.62-2.53 (m, 1H), 2.22-2.04 (m, 4H), 2.04-1.89 (m, 2H), 1.00-0.87 (m, 2H), 0.68-0.57 (m, 2H)

LCMS (m/z [M+H]⁺): 336.2

### Example 43: Synthesis of 2-benzamido-5-chloro-N-(2,6-dioxopiperidin-3-yl)thiophene-3-carboxamide (45)

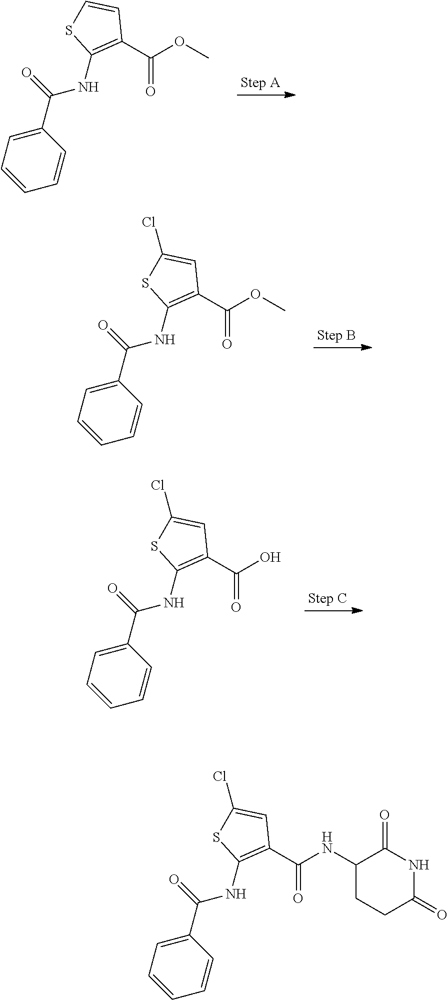 [SMILES: COC(=O)c1ccsc1NC(=O)c1ccccc1 ; COC(=O)c1cc(Cl)sc1NC(=O)c1ccccc1 ; O=C(Nc1sc(Cl)cc1C(=O)O)c1ccccc1 ; O=C1CCC(NC(=O)c2cc(Cl)sc2NC(=O)c2ccccc2)C(=O)N1]

Step A: methyl 2-benzamido-5-chlorothiophene-3-carboxylate was synthesized using Example

Method 1, above (69% yield), using methyl 2-benzamidothiophene-3-carboxylate as a starting material.

Step B: 2-benzamido-5-chlorothiophene-3-carboxylic acid was synthesized using Example Method 2, above (70% yield), using methyl 2-benzamido-5-chlorothiophene-3-carboxylate as a starting material.

Step C: 2-benzamido-5-chloro-N-(2,6-dioxopiperidin-3-yl)thiophene-3-carboxamide was synthesized using the general procedure shown in Reaction Scheme 1 and Synthetic Conditions A, above (50% yield), and 2-benzamido-5-chlorothiophene-3-carboxylic acid as a starting material.

¹H NMR (500 MHz, DMSO) δ 13.20 (s, 1H), 10.92 (s, 1H), 8.86-8.73 (m, 1H), 8.00-7.84 (m, 2H), 7.72-7.50 (m, 4H), 4.95-4.81 (m, 1H), 2.89-2.74 (m, 1H), 2.67-2.56 (m, 1H), 2.22-2.08 (m, 1H), 2.06-1.93 (m, 1H)

LCMS (m/z [M+H]⁺): 392.2

### Example 44: Synthesis of 5-chloro-N-(2,6-dioxopiperidin-3-yl)-2-(2-phenylacetamido)thiophene-3-carboxamide (46)

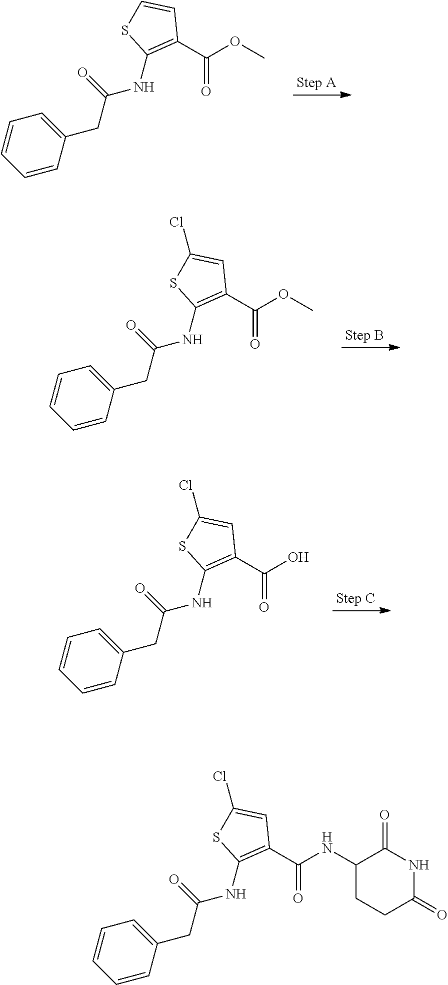 [SMILES: COC(=O)c1ccsc1NC(=O)Cc1ccccc1 ; COC(=O)c1cc(Cl)sc1NC(=O)Cc1ccccc1 ; O=C(Cc1ccccc1)Nc1sc(Cl)cc1C(=O)O ; O=C1CCC(NC(=O)c2cc(Cl)sc2NC(=O)Cc2ccccc2)C(=O)N1]

Step A: Methyl 5-chloro-2-(2-phenylacetamido)thiophene-3-carboxylate was synthesized using Example Method 1, above (75% yield), using methyl 2-(2-phenylacetamido)thiophene-3-carboxylate as a starting material.

Step B: 5-chloro-2-(2-phenylacetamido)thiophene-3-carboxylic acid was synthesized using Example Method 2, above (82% yield), using methyl 5-chloro-2-(2-phenylacetamido)thiophene-3-carboxylate as a starting material.

Step C: 5-chloro-N-(2,6-dioxopiperidin-3-yl)-2-(2-phenylacetamido)thiophene-3-carboxamide was synthesized using the general procedure shown in Reaction Scheme 1 and Synthetic Conditions A, above (15% yield), with 5-chloro-2-(2-phenylacetamido)thiophene-3-carboxylic acid as a starting material.

¹H NMR (400 MHz, DMSO) δ 11.94 (s, 1H), 10.91 (s, 1H), 8.72-8.54 (m, 1H), 7.66-7.45 (m, 2H), 7.43-7.21 (m, 4H), 4.82-4.64 (m, 1H), 3.90 (s, 2H), 2.89-2.71 (m, 1H), 2.61-2.53 (m, 1H), 2.17-2.02 (m, 1H), 2.01-1.88 (m, 1H)

LCMS (m/z [M+H]⁺): 406.2

### Example 45: Synthesis of 2-cyclopropyl-N-(2,6-dioxopiperidin-3-yl)furan-3-carboxamide (47)

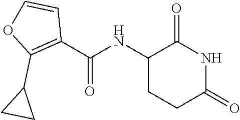 [SMILES: O=C1CCC(NC(=O)c2ccoc2C2CC2)C(=O)N1]

This compound was synthesized using the general procedure shown in Reaction Scheme 1 and Synthetic Conditions C, above (53% yield) using 2-cyclopropylfuran-3-carboxylic acid (20 mg) as a starting material.

¹H NMR (500 MHz, DMSO) δ 10.83 (s, 1H), 8.24 (d, J=8.5 Hz, 1H), 7.42 (d, J=2.0 Hz, 1H), 6.83 (d, J=2.1 Hz, 1H), 4.70 (ddd, J=12.4, 8.3, 5.3 Hz, 1H), 2.87 (tt, J=8.5, 5.2 Hz, 1H), 2.78 (ddd, J=17.4, 13.4, 5.6 Hz, 1H), 2.60-2.51 (m, 1H), 2.10 (qd, J=13.0, 4.4 Hz, 1H), 1.98-1.92 (m, 1H), 1.00-0.95 (m, 2H), 0.91-0.87 (m, 2H).

LCMS (m/z [M+H]⁺): 263.2

### Example 46: Synthesis of tert-butyl (3-((2,6-dioxopiperidin-3-yl)carbamoyl)furan-2-yl)(methyl)carbamate (48)

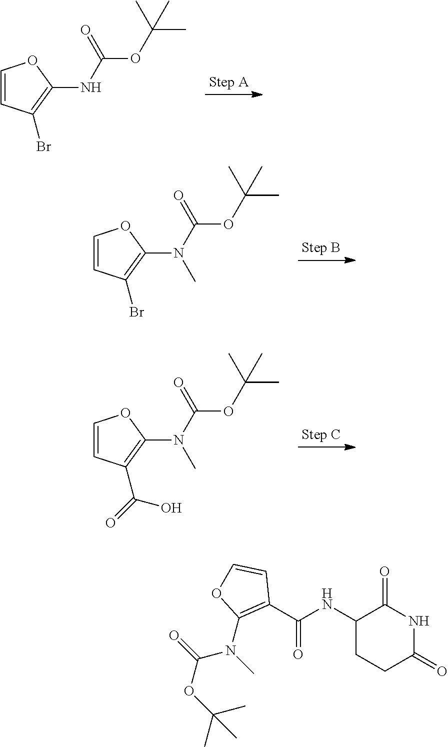 [SMILES: CC(C)(C)OC(=O)Nc1occc1Br ; CN(C(=O)OC(C)(C)C)c1occc1Br ; CN(C(=O)OC(C)(C)C)c1occc1C(=O)O ; CN(C(=O)OC(C)(C)C)c1occc1C(=O)NC1CCC(=O)NC1=O]

Step A: To a solution of tert-butyl (3-bromofuran-2-yl)carbamate (2 g, 7.6 mmol) in DMF (40 ml) was added sodium hydride (0.28 g, 11.5 mmol) at 0° C. under nitrogen and the reaction mixture was stirred at RT for 1 h. It was then re-cooled at 0° C., methyl iodide (1.42 ml, 23 mmol) was added and the reaction mixture was stirred for an additional 1 h at RT. The reaction mixture was diluted with ethyl acetate, washed with water and brine, dried over Na₂SO₄, concentrated under reduced pressure and purified by flash column chromatography to give tert-butyl (3-bromofuran-2-yl)(methyl)carbamate 1.27 g (60% yield).

Step B: n-butyllithium (3.37 ml, 5.43 mmol, 1.6 M in hexane) was added slowly to a THF (30 ml) solution of tert-butyl (3-bromofuran-2-yl)(methyl)carbamate (1.5 g, 5.43 mmol) at −78° C. under nitrogen. After 15 min of stirring, a stream of dry CO₂ was bubbled into the solution for 30 min. The reaction was quenched with 1M HCl (10 ml), extracted with DCM, fried over Na₂SO₄, concentrated under reduced pressure to give 2-((tert-butoxycarbonyl)(methyl)amino)furan-3-carboxylic acid 500 mg (38% yield).

Step C: tert-butyl (3-((2,6-dioxopiperidin-3-yl)carbamoyl)furan-2-yl)(methyl)carbamate was synthesized using the general procedure shown in Reaction Scheme 1 and Synthetic Conditions B, above, (59% yield) using 2-((tert-butoxycarbonyl)(methyl)amino)furan-3-carboxylic acid (20 mg) as a starting material.

¹H NMR (500 MHz, DMSO) δ 10.84 (s, 1H), 8.28 (d, J=8.3 Hz, 1H), 7.52 (d, J=2.3 Hz, 1H), 6.89 (d, J=2.3 Hz, 1H), 4.70 (ddd, J=12.2, 8.2, 5.3 Hz, 1H), 3.03 (s, 3H), 2.78 (ddd, J=17.3, 13.3, 5.5 Hz, 1H), 2.54 (dd, J=4.5, 2.9 Hz, 1H), 2.13-2.00 (m, 1H), 1.94 (dtd, J=10.2, 5.5, 2.9 Hz, 1H), 1.45-1.19 (m, 9H).

LCMS (m/z [M−H]⁻): 350.3

### Example 47: Synthesis of tert-butyl ((5-((2,6-dioxopiperidin-3-yl)carbamoyl)thiophen-2-yl)methyl)carbamate (49)

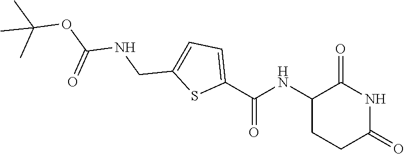 [SMILES: CC(C)(C)OC(=O)NCc1ccc(C(=O)NC2CCC(=O)NC2=O)s1]

This compound was synthesized using the general procedure shown in Reaction Scheme 1 and Synthetic Conditions C, above, (24% yield) using 5-[(tert-butoxycarbonylamino)methyl]thiophene-2-carboxylic acid (23 mg) as a starting material.

¹H NMR (500 MHz, DMSO) δ 10.85 (s, 1H), 8.68 (d, J=8.4 Hz, 1H), 7.59 (d, J=3.7 Hz, 1H), 6.94 (d, J=3.7 Hz, 1H), 4.70 (ddd, J=12.7, 8.4, 5.3 Hz, 1H), 4.27 (d, J=6.0 Hz, 2H), 2.78 (ddd, J=17.4, 13.3, 5.5 Hz, 1H), 2.52-2.51 (m, 2H), 2.15-2.04 (m, 1H), 2.00-1.92 (m, 1H), 1.40 (s, 9H).

LCMS (m/z [M−H]⁻): 366.1

### Example 48: Synthesis of 5-(aminomethyl)-N-(2,6-dioxopiperidin-3-yl)thiophene-2-carboxamide (50)

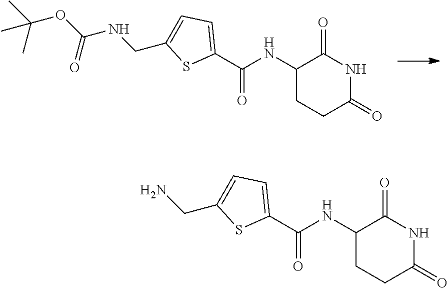 [SMILES: CC(C)(C)OC(=O)NCc1ccc(C(=O)NC2CCC(=O)NC2=O)s1 ; NCc1ccc(C(=O)NC2CCC(=O)NC2=O)s1]

To the suspension of tert-butyl ((5-((2,6-dioxopiperidin-3-yl)carbamoyl)thiophen-2-yl)methyl)carbamate (5 mg, 0.014 mmol, 1 eq) in DCM (0.1 mL) was added TFA (20 μL) and mixture was stirred for 2 days at RT. The reaction mixture was evaporated under reduced pressure and suspended in ACN, then 4N HCl in 1,4-dioxane was added (50 μL). Concentration under reduced pressure gave 5-(aminomethyl)-N-(2,6-dioxopiperidin-3-yl)thiophene-2-carboxamide hydrochloride (92% yield).

¹H NMR (500 MHz, DMSO) δ 10.88 (s, 1H), 8.86 (d, J=8.4 Hz, 1H), 8.29 (s, 3H), 7.71 (d, J=3.8 Hz, 1H), 7.25 (d, J=3.8 Hz, 1H), 4.77-4.65 (m, 1H), 4.27 (s, 2H), 2.79 (ddd, J=17.4, 13.4, 5.5 Hz, 1H), 2.52-2.51 (m, 1H), 2.12 (qd, J=13.2, 4.7 Hz, 1H), 1.97 (dddd, J=10.9, 8.2, 5.4, 2.8 Hz, 1H).

LCMS (m/z [M−1-1]+): 268.0

### Example 49: tert-butyl ((5-((2,6-dioxopiperidin-3-yl)carbamoyl)thiophen-3-yl)methyl)carbamate (51)

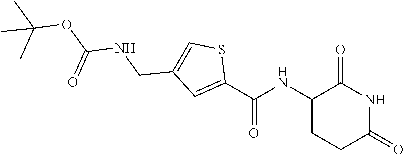 [SMILES: CC(C)(C)OC(=O)NCc1csc(C(=O)NC2CCC(=O)NC2=O)c1]

This compound was synthesized using the general procedure shown in Reaction Scheme 1 and Synthetic Conditions C, above, (31% yield) using 4-((tert-butoxycarbonyl)aminomethyl)thiophene-2-carboxylic acid (37.8 mg) as a starting material.

1H NMR (500 MHz, DMSO) δ 10.85 (s, 1H), 8.77 (d, J=8.4 Hz, 1H), 7.65 (d, J=1.3 Hz, 1H), 7.47 (d, J=1.2 Hz, 1H), 4.71 (ddd, J=12.6, 8.4, 5.3 Hz, 1H), 4.09 (d, J=5.9 Hz, 2H), 2.78 (ddd, J=17.4, 13.3, 5.5 Hz, 1H), 2.52-2.51 (m, 2H), 2.16-2.04 (m, 1H), 1.96 (dddd, J=10.9, 8.2, 5.4, 2.9 Hz, 1H), 1.39 (s, 9H).

LCMS (m/z [M−H]⁻): 366.2

### Example 50: 4-(aminomethyl)-N-(2,6-dioxopiperidin-3-yl)thiophene-2-carboxamide (52)

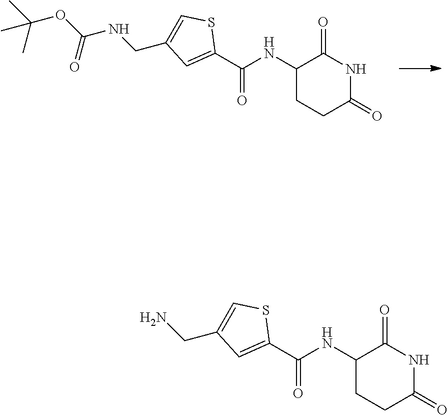 [SMILES: CC(C)(C)OC(=O)NCc1csc(C(=O)NC2CCC(=O)NC2=O)c1 ; NCc1csc(C(=O)NC2CCC(=O)NC2=O)c1]

To the suspension of tert-butyl ((5-((2,6-dioxopiperidin-3-yl)carbamoyl)thiophen-3-yl)methyl)carbamate (15.8 mg, 0.043 mmol) in DCM (0.5 mL) was added TFA (0.1 mL) and mixture was stirred for 2 days at RT. Mixture was evaporated and purified by HPLC to give 4-(aminomethyl)-N-(2,6-dioxopiperidin-3-yl)thiophene-2-carboxamide formate (63% yield).

¹H NMR (500 MHz, DMSO) δ 10.85 (s, 1H), 8.78 (d, J=8.4 Hz, 1H), 7.78 (d, J=1.2 Hz, 1H), 7.71 (s, 1H), 4.73 (ddd, J=12.6, 8.4, 5.4 Hz, 1H), 3.93 (s, 2H), 2.79 (ddd, J=17.4, 13.3, 5.5 Hz, 1H), 2.56 (dd, J=4.4, 3.0 Hz, 1H), 2.52 (dd, J=4.3, 2.5 Hz, 1H), 2.11 (qd, J=12.9, 4.5 Hz, 1H), 1.96 (dddd, J=10.9, 8.2, 5.4, 2.9 Hz, 1H).

LCMS (m/z [M+H]⁺): 268.1

### Example 51: Synthesis of N-(2,6-dioxopiperidin-3-yl)-4-hydroxythiophene-2-carboxamide (53)

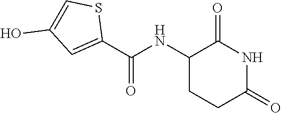 [SMILES: O=C1CCC(NC(=O)c2cc(O)cs2)C(=O)N1]

This compound was synthesized using the general procedure shown in Reaction Scheme 1 and Synthetic Conditions D, above, (20% yield) using 4-hydroxythiophene-2-carboxylic acid (20 mg) as a starting material.

¹H NMR (500 MHz, DMSO) δ 10.86 (s, 1H), 9.75 (s, 1H), 8.67 (d, J=8.4 Hz, 1H), 7.35 (d, J=1.7 Hz, 1H), 6.55 (d, J=1.7 Hz, 1H), 4.71 (ddd, J=12.5, 8.4, 5.4 Hz, 1H), 2.85-2.73 (m, 1H), 2.58-2.52 (m, 1H), 2.10 (qd, J=13.0, 4.5 Hz, 1H), 2.02-1.92 (m, 1H).

LCMS (m/z [M−H]⁻): 253.1

### Example 52: Synthesis of N-(2,6-dioxopiperidin-3-yl)-4-methoxythiophene-3-carboxamide (54)

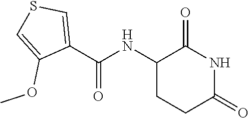 [SMILES: COc1cscc1C(=O)NC1CCC(=O)NC1=O]

This compound was synthesized using the general procedure shown in Reaction Scheme 1 and Synthetic Conditions D, above, (32% yield) using 4-methoxythiophene-3-carboxylic acid (20 mg) as a starting material.

¹H NMR (500 MHz, DMSO) δ 10.89 (s, 1H), 8.12 (d, J=7.4 Hz, 1H), 8.09 (d, J=3.6 Hz, 1H), 6.81 (d, J=3.6 Hz, 1H), 4.72 (dt, J=10.3, 7.5 Hz, 1H), 3.89 (s, 3H), 2.85-2.72 (m, 1H), 2.54 (t, J=3.5 Hz, 1H), 2.15-2.05 (m, 2H).

LCMS (m/z [M+H]⁺): 269.0

### Example 53: Synthesis of 5-(aminomethyl)-N-(2,6-dioxopiperidin-3-yl)-4-methoxythiophene-3-carboxamide (55)

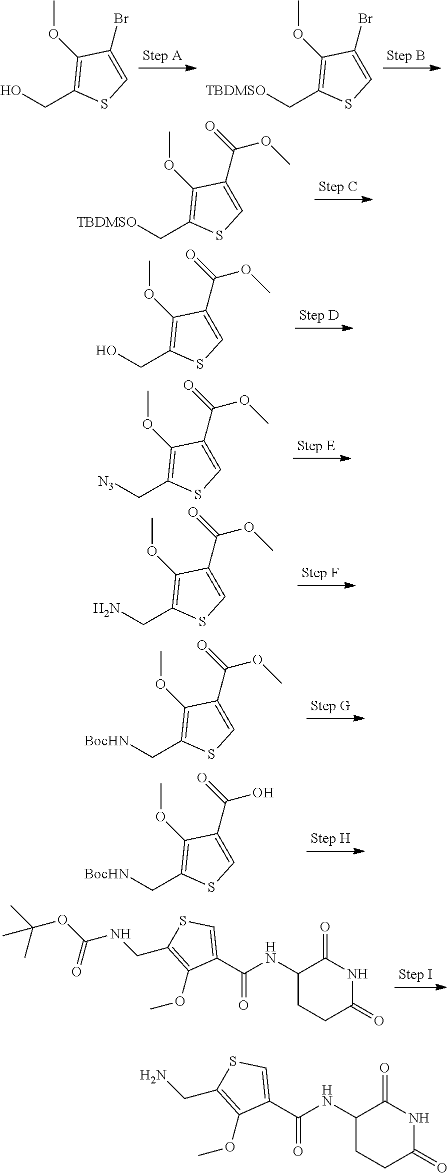

Step A: (4-Bromo-3-methoxythiophen-2-yl)methanol (0.8 g, 3.58 mmol) was dissolved in DCM (10 mL). TBDMSCl (1.08 g, 7.17 mmol) and imidazole (0.6 g, 8.96 mmol) were added and the reaction mixture was stirred at RT for 48 h, diluted, washed with water, dried over Na₂SO₄, concentrated under reduced pressure and purified by flash column chromatography to give ((4-bromo-3-methoxythiophen-2-yl)methoxy)(tert-butyl)dimethylsilane (82% yield).

Step B: ((4-bromo-3-methoxythiophen-2-yl)methoxy)(tert-butyl)dimethylsilane (1.5 g, 4.45 mmol) was dissolved in THF (20 mL) and cooled to −78° C. n-BuLi (3.7 mL, 6.67 mmol) was added dropwise and the reaction mixture was stirred for 30 min. Methyl chloroformate (0.62 mL, 8.0 mmol) was added and stirring was continued for 2 h at −78° C. Ammonium chloride solution was added, the product was extracted with ethyl acetate, dried over Na₂SO₄, concentrated under reduced pressure and purified by flash column chromatography to give methyl 5-(((tert-butyldimethylsilyl)oxy)methyl)-4-methoxythiophene-3-carboxylate (51% yield).

Step C: methyl 5-(((tert-butyldimethylsilyl)oxy)methyl)-4-methoxythiophene-3-carboxylate (0.8 g, 2.52 mmol) was dissolved in THF (10 mL) and TBAF (1M solution in THF) (5.06 mL, 5.0 mmol) was added at 0° C. Reaction mixture was stirred at rt for 4 h, diluted with ethyl acetate and washed with water. Organic phase was dried over Na₂SO₄ and concentrated under reduced pressure to give methyl 5-(hydroxymethyl)-4-methoxythiophene-3-carboxylate (88% yield).

Step D: methyl 5-(hydroxymethyl)-4-methoxythiophene-3-carboxylate (0.2 g, 1.0 mmol) was dissolved in toluene (3 mL) and cooled to 0° C. DBU (0.19 mL, 1.3 mmol), and DPPA (0.26 mL, 1.2 mmol) were added and reaction mixture was stirred at RT for 16 h. The mixture was diluted with ethyl acetate, washed with water, dried over Na₂SO₄, concentrated under reduced pressure and purified by flash column chromatography to give methyl 5-(azidomethyl)-4-methoxythiophene-3-carboxylate (78% yield).

Step E: methyl 5-(azidomethyl)-4-methoxythiophene-3-carboxylate (40 mg, 0.176 mmol) was dissolved in MeOH (5 mL) and 10% Pd/C (20 mg) was added. Reaction mixture was stirred under H₂ atmosphere at RT for 3 h, filtered through celite bed and concentrated under reduced pressure to give methyl 5-(aminomethyl)-4-methoxythiophene-3-carboxylate that was used directly in the next step.

Step F: methyl 5-(aminomethyl)-4-methoxythiophene-3-carboxylate (215 mg, 1.06 mmol) was dissolved in a dioxane-water (1:1; 6 mL). triethylamine (0.22 mL, 1.6 mmol) and Boc₂O (0.29 mL, 1.28 mmol) were added and the reaction mixture was stirred at RT for 18 h, diluted with ethyl acetate, washed with water, dried over Na₂SO₄, concentrated under reduced pressure and purified by flash column chromatography to give methyl 5-(((tert-butoxycarbonyl)amino)methyl)-4-methoxythiophene-3-carboxylate (38% yield, 2 steps).

Step G: To 5-(((tert-butoxycarbonyl)amino)methyl)-4-methoxythiophene-3-carboxylate (200.0 mg, 0.66 mmol) in THF (1.0 mL) was added methanol (1.0 mL) and 50% aqueous NaOH (2 mL), the reaction mixture was stirred at RT for 16 h, diluted with water and acidified with citric acid. The product was extracted with ethyl acetate, concentrated and triturated with diethyl ether to give 5-(((tert-butoxycarbonyl)amino)methyl)-4-methoxythiophene-3-carboxylic acid (83% yield).

Step H: tert-butyl ((4-((2,6-dioxopiperidin-3-yl)carbamoyl)-3-methoxythiophen-2-yl)methyl)carbamate was synthesized using the general procedure shown in Reaction Scheme 1 and Synthetic Conditions B, above (23% yield), and 5-(((tert-butoxycarbonyl)amino)methyl)-4-methoxythiophene-3-carboxylic acid (30 mg) as a starting material.

¹H NMR (500 MHz, DMSO) δ 10.86 (s, 1H), 8.67 (d, J=8.4 Hz, 1H), 7.64 (s, 1H), 7.42 (t, J=5.8 Hz, 1H), 4.79-4.63 (m, 1H), 4.15 (d, J=6.0 Hz, 2H), 3.80 (s, 3H), 2.79 (ddd, J=17.4, 13.2, 5.5 Hz, 1H), 2.60-2.52 (m, 1H), 2.08 (qd, J=12.9, 4.4 Hz, 1H), 2.03-1.90 (m, 1H), 1.39 (s, 9H).

LCMS (m/z [M−H]⁻): 396.0

Step I: tert-butyl ((4-((2,6-dioxopiperidin-3-yl)carbamoyl)-3-methoxythiophen-2-yl)methyl) carbamate (5 mg) was dissolved in 2 mL of TFA and the solution was stirred at RT for 2 h. The volatiles were removed under reduced pressure to give 5-(aminomethyl)-N-(2,6-dioxopiperidin-3-yl)-4-methoxythiophene-3-carboxamide trifluoroacetate (87% yield).

¹H NMR (500 MHz, DMSO) δ 10.88 (s, 1H), 8.88 (d, J=8.3 Hz, 1H), 8.18 (s, 3H), 7.77 (s, 1H), 4.79-4.65 (m, 1H), 4.10 (q, J=5.4 Hz, 2H), 2.79 (ddd, J=17.5, 13.3, 5.5 Hz, 1H), 2.54 (ddd, J=9.0, 6.1, 2.2 Hz, 1H), 2.17-2.07 (m, 1H), 2.03-1.94 (m, 1H).

### Example 54: Synthesis of tert-butyl (3-((2,6-dioxopiperidin-3-yl)carbamoyl)thiophen-2-yl)(methyl)carbamate (56)

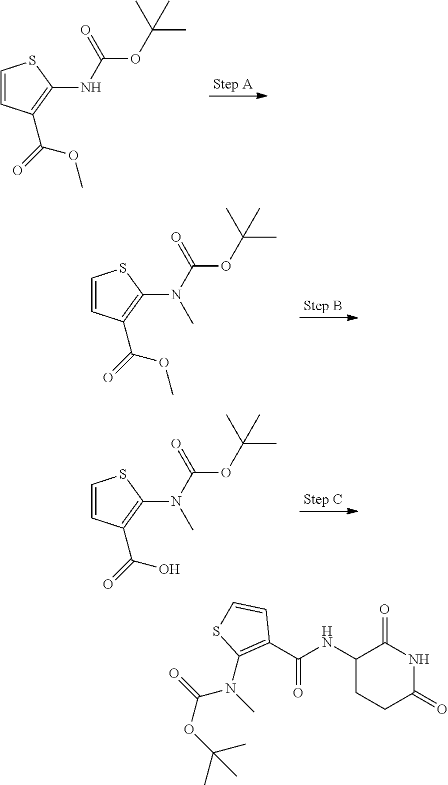 [SMILES: COC(=O)c1ccsc1NC(=O)OC(C)(C)C ; COC(=O)c1ccsc1N(C)C(=O)OC(C)(C)C ; CN(C(=O)OC(C)(C)C)c1sccc1C(=O)O ; CN(C(=O)OC(C)(C)C)c1sccc1C(=O)NC1CCC(=O)NC1=O]

Step A: methyl 2-((tert-butoxycarbonyl)amino)thiophene-3-carboxylate (100 mg, 1 eq.) was added to a stirred mixture of 60% NaH (1.2 eq.) suspended in mineral oil in dry DMF in an inert atmosphere, next was added Mel (1.2 eq.). The resulting mixture was stirred at room temperature for 18 hours, solvent was removed under reduced pressure and the residue was purified by flash column chromatography to give methyl 2-((tert-butoxycarbonyl)(methyl)amino)thiophene-3-carboxylate (49% yield).

Step B: 1M NaOH in H₂O (10 eq.) was added to a solution of methyl 2-((tert-butoxycarbonyl)(methyl)amino)thiophene-3-carboxylate (52.0 mg, 1. eq.) in methanol and was stirring at room temperature for 18 hours. When the reaction was completed, to the acidified with 1M HCl, concentrated under reduced pressure and partitioned between ethyl acetate and water. Organic layer was washed with brine, dried over Na₂SO₄ and evaporated. 2-((tert-butoxycarbonyl)(methyl)amino)thiophene-3-carboxylic acid (100%) was used without further purification in the next step.

Step C: tert-butyl (3-((2,6-dioxopiperidin-3-yl)carbamoyl)thiophen-2-yl)(methyl)carbamate was synthesized using the general procedure shown in Reaction Scheme 1 and Synthetic Conditions C, above, (57% yield) using 2-((tert-butoxycarbonyl)(methyl)amino)thiophene-3-carboxylic acid (49.4 mg) as a starting material.

¹H NMR (500 MHz, DMSO) δ 10.84 (s, 1H), 8.30 (d, J=8.2 Hz, 1H), 7.38 (d, I=5.8 Hz, 1H), 7.25 (d, J=5.9 Hz, 1H), 4.70 (ddd, J=13.0, 8.1, 5.3 Hz, 1H), 3.10 (s, 3H), 2.78 (ddd, J=17.3, 13.3, 5.6 Hz, 1H), 2.59-2.53 (m, 1H), 2.11-2.01 (m, 1H), 1.95 (dtd, J=12.8, 5.4, 2.9 Hz, 1H), 1.32 (s, 9H).

LCMS (m/z [M-Boc+H]+): 268.0

### Example 55: Synthesis of N-(2,6-dioxopiperidin-3-yl)-5-phenylthiophene-3-carboxamide (57)

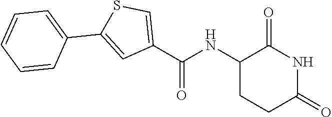 [SMILES: O=C1CCC(NC(=O)c2csc(-c3ccccc3)c2)C(=O)N1]

This compound was synthesized using the general procedure shown in Reaction Scheme 1 and Synthetic Conditions D, above, (50% yield) using 5-phenylthiophene-3-carboxylic acid (16.7 mg) as a starting material.

¹H NMR (500 MHz, DMSO) δ 10.87 (s, 1H), 8.64 (d, J=8.4 Hz, 1H), 8.15 (d, J=1.4 Hz, 1H), 7.89 (d, J=1.4 Hz, 1H), 7.71-7.65 (m, 2H), 7.51-7.42 (m, 2H), 7.40-7.33 (m, 1H), 4.83-4.73 (m, 1H), 2.81 (ddd, J=18.6, 13.2, 5.6 Hz, 1H), 2.60-2.53 (m, 1H), 2.18-2.04 (m, 1H), 2.00 (ddt, J=11.0, 8.4, 4.2 Hz, 1H).

LCMS (m/z [M]⁺): 315.0

### Example 56: Synthesis of 2-acetamido-N-(2,6-dioxopiperidin-3-yl)-5-phenylthiophene-3-carboxamide (58)

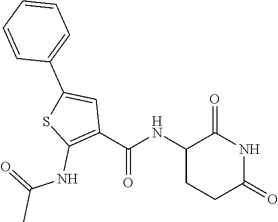 [SMILES: CC(=O)Nc1sc(-c2ccccc2)cc1C(=O)NC1CCC(=O)NC1=O]

This compound was synthesized using the general procedure shown in Reaction Scheme 1 and Synthetic Conditions C, above, (88% yield) using 2-acetamido-5-phenylthiophene-3-carboxylic acid (20 mg) as a starting material.

¹H NMR (500 MHz, DMSO) δ 11.84 (s, 1H), 10.92 (s, 1H), 8.72 (d, J=8.2 Hz, 1H), 7.87 (s, 1H), 7.62-7.55 (m, 2H), 7.48-7.39 (m, 2H), 7.35-7.26 (m, 1H), 4.83-4.70 (m, 1H), 2.81 (ddd, J=17.2, 13.4, 5.5 Hz, 1H), 2.58 (dt, J=16.8, 3.8 Hz, 1H), 2.24 (s, 3H), 2.17 (qd, J=12.9, 4.5 Hz, 1H), 2.01 (dtd, J=12.9, 5.4, 2.8 Hz, 1H).

LCMS (m/z [M+H]⁺): 372.3

### Example 57: Synthesis of 2-benzamido-N-(2,6-dioxopiperidin-3-yl)-5-phenylthiophene-3-carboxamide (59)

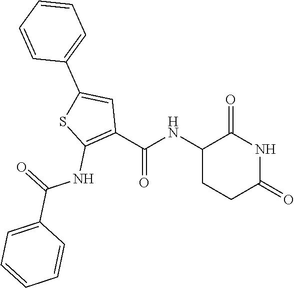 [SMILES: O=C1CCC(NC(=O)c2cc(-c3ccccc3)sc2NC(=O)c2ccccc2)C(=O)N1]

This compound was synthesized using the general procedure shown in Reaction Scheme 1 and Synthetic Conditions C, above, (83% yield) using 2-benzamido-5-phenylthiophene-3-carboxylic acid (20 mg) as a starting material.

¹H NMR (500 MHz, DMSO) δ 13.21 (s, 1H), 10.96 (s, 1H), 8.87 (d, J=8.4 Hz, 1H), 7.99 (s, 1H), 7.97-7.91 (m, 2H), 7.74-7.68 (m, 1H), 7.68-7.61 (m, 4H), 7.51-7.43 (m, 2H), 7.38-7.29 (m, 1H), 4.94 (ddd, J=13.0, 8.3, 5.3 Hz, 1H), 2.90-2.79 (m, 1H), 2.60 (dt, J=17.7, 3.8 Hz, 1H), 2.23-2.12 (m, 1H), 2.07-2.02 (m, 1H).

LCMS (m/z [M+H]⁺): 434.2

### Example 58: Synthesis of N-(2,6-dioxopiperidin-3-yl)-5-phenyl-2-(2-(pyrrolidin-1-yl)acetamido)thiophene-3-carboxamide (60)

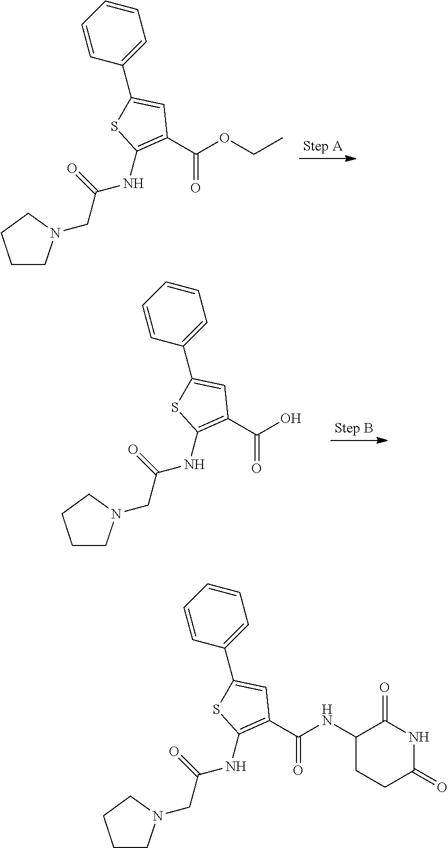 [SMILES: CCOC(=O)c1cc(-c2ccccc2)sc1NC(=O)CN1CCCC1 ; O=C(CN1CCCC1)Nc1sc(-c2ccccc2)cc1C(=O)O ; O=C1CCC(NC(=O)c2cc(-c3ccccc3)sc2NC(=O)CN2CCCC2)C(=O)N1]

Step A: To the solution of ethyl 5-phenyl-2-(2-(pyrrolidin-1-yl)acetamido)thiophene-3-carboxylate (20 mg, 0.056 mmol) in EtOH (0.6 mL) was added H₂O (0.1 mL) followed by NaOH (4 eq). Reaction was stirred at 50° C. for 3 h. The reaction mixture was acidified with 1M HCl, concentrated under reduced pressure and the crude 5-phenyl-2-(2-(pyrrolidin-1-yl)acetamido)thiophene-3-carboxylic acid was used directly in the next step (98% yield).

Step B: N-(2,6-dioxopiperidin-3-yl)-5-phenyl-2-(2-(pyrrolidin-1-yl)acetamido)thiophene-3-carboxamide was synthesized using the general procedure shown in Reaction Scheme 1 and Synthetic Conditions C, above, (15% yield) using 5-phenyl-2-(2-(pyrrolidin-1-yl)acetamido)thiophene-3-carboxylic acid (18 mg) as a starting material.

¹H NMR (500 MHz, DMSO) δ 12.51 (s, 1H), 10.91 (s, 1H), 8.61 (d, J=8.5 Hz, 1H), 8.14 (s, 1H), 7.88 (s, 1H), 7.62-7.57 (m, 2H), 7.47-7.41 (m, 2H), 7.33-7.28 (m, 1H), 4.87 (ddd, J=12.4, 8.5, 5.5 Hz, 1H), 3.41 (s, 2H), 2.91-2.75 (m, 1H), 2.63 (td, J=4.9, 4.4, 2.3 Hz, 4H), 2.57 (ddd, J=17.3, 4.4, 2.9 Hz, 1H), 2.16-2.06 (m, 1H), 2.06-1.98 (m, 1H), 1.82-1.74 (m, 4H).

LCMS (m/z [M−1-1]1: 441.1

### Example 59: Synthesis of tert-butyl ((3-((2,6-dioxopiperidin-3-yl)carbamoyl)thiophen-2-yl)methyl)carbamate (63)

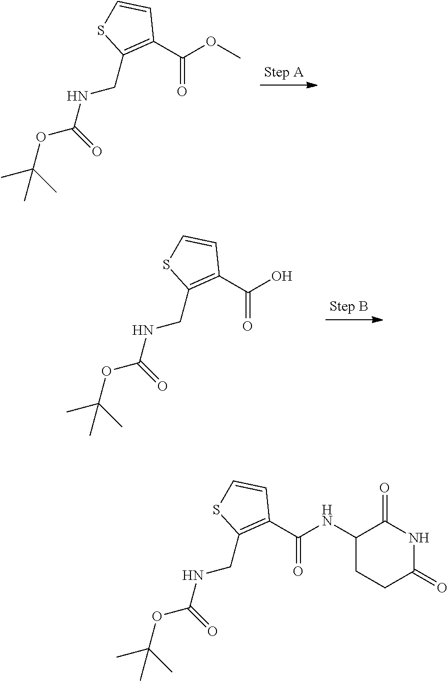 [SMILES: COC(=O)c1ccsc1CNC(=O)OC(C)(C)C ; CC(C)(C)OC(=O)NCc1sccc1C(=O)O ; CC(C)(C)OC(=O)NCc1sccc1C(=O)NC1CCC(=O)NC1=O]

Step A: To the solution of methyl 2-(((tert-butoxycarbonyl)amino)methyl)thiophene-3-carboxylate (35 mg, 0.129 mmol, 1. eq.) in THF (1.0 mL) was added H₂O (0.3 mL) followed by NaOH (6 eq). Reaction was stirred at RT for 18 h and at 50° C. for 4 h. The reaction mixture was acidified with 1M HCl, extracted with EtOAc, dried over Na₂SO₄ and concentrated to give 2-(((tert-butoxycarbonyl)amino)methyl)thiophene-3-carboxylic acid (86% yield).

Step B: Tert-butyl ((3-((2,6-dioxopiperidin-3-yl)carbamoyl)thiophen-2-yl)methyl)carbamate was synthesized using the general procedure shown in Reaction Scheme 1 and Synthetic Conditions C, above, (37% yield) using 2-(((tert-butoxycarbonyl)amino)methyl)thiophene-3-carboxylic acid (30 mg) as a starting material.

¹H NMR (500 MHz, DMSO) δ 10.84 (s, 1H), 8.47 (d, J=8.3 Hz, 1H), 7.39 (s, 2H), 4.78-4.67 (m, 1H), 4.57 (d, J=6.7 Hz, 2H), 2.78 (ddd, J=17.4, 13.3, 5.5 Hz, 1H), 2.57-2.51 (m, 2H), 2.10 (qd, J=13.0, 4.5 Hz, 1H), 2.03-1.91 (m, 1H), 1.40 (s, 9H).

LCMS (m/z [M−H]⁻): 366.0

### Example 60: Synthesis of 2-(aminomethyl)-N-(2,6-dioxopiperidin-3-yl)thiophene-3-carboxamide (64)

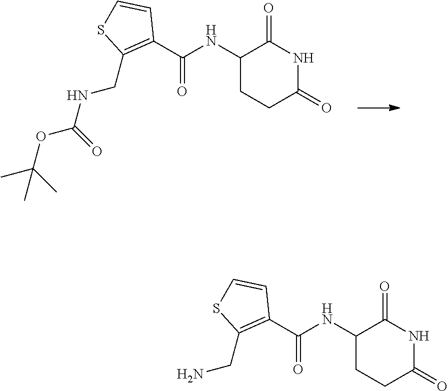 [SMILES: CC(C)(C)OC(=O)NCc1sccc1C(=O)NC1CCC(=O)NC1=O ; NCc1sccc1C(=O)NC1CCC(=O)NC1=O]

To the suspension of tert-butyl ((3-((2,6-dioxopiperidin-3-yl)carbamoyl)thiophen-2-yl)methyl)carbamate (12.7 mg, 0.035 mmol, 1 eq) in DCM (0.2 mL) was added TFA (50 μl) and mixture was stirred for 2 days at RT. The reaction mixture was evaporated under reduced pressure and suspended in ACN, then 4N HCl in 1,4-dioxane was added (50 μL). Concentration under reduced pressure gave 2-(aminomethyl)-N-(2,6-dioxopiperidin-3-yl)thiophene-3-carboxamide hydrochloride (98% yield).

¹H NMR (500 MHz, DMSO) δ 10.91 (s, 1H), 8.90 (d, J=8.3 Hz, 1H), 8.25 (s, 3H), 7.68 (d, J=5.4 Hz, 1H), 7.59 (d, J=5.4 Hz, 1H), 4.82-4.70 (m, 1H), 2.80 (ddd, J=17.4, 13.4, 5.5 Hz, 1H), 2.60-2.53 (m, 1H), 2.15 (qd, J=13.0, 4.5 Hz, 1H), 1.98 (dddd, J=10.8, 8.2, 5.3, 2.9 Hz, 1H).

LCMS (m/z [M+H]⁺): 268.0

### Example 61: Synthesis of N-(2,5-dioxopyrrolidin-3-yl)-2-methoxythiophene-3-carboxamide (65)

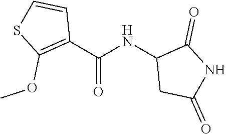 [SMILES: COc1sccc1C(=O)NC1CC(=O)NC1=O]

To the mixture of 2-methoxythiophene-3-carboxylic acid (20.0 mg, 0.126 mmol, 1.000 eq) and CDI (30.8 mg, 0.189 mmol, 1.500 eq) was added DMF (1.0 mL) and the reaction mixture was stirred for 1 h at 50° C. After cooling to RT 3-aminopyrrolidine-2,5-dione hydrochloride (22.8 mg, 0.151 mmol, 1.200 eq) was added and the reaction mixture was stirred overnight. The solvent was removed under reduced pressure and the residue was purified by preparative TLC (78% yield).

1H NMR (500 MHz, DMSO) δ 11.19 (s, 1H), 8.14 (d, J=7.7 Hz, 1H), 7.08 (d, J=5.9 Hz, 1H), 6.83 (d, J=6.0 Hz, 1H), 4.65 (ddd, J=9.3, 7.8, 5.8 Hz, 1H), 4.05 (s, 3H), 2.89 (dd, J=17.4, 9.4 Hz, 1H), 2.60 (dd, J=17.4, 5.8 Hz, 1H).

LCMS (m/z [M+H]⁺): 254.8

### Example 62: Synthesis of 2-cyclopropyl-N-(2,6-dioxopiperidin-3-yl)thiophene-3-carboxamide (66)

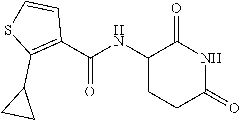 [SMILES: O=C1CCC(NC(=O)c2ccsc2C2CC2)C(=O)N1]

This compound was synthesized using the general procedure shown in Reaction Scheme 1 and Synthetic Conditions D, above, (72% yield) using 2-(cyclopropyl)thiophene-3-carboxylic acid (20 mg) as a starting material.

¹H NMR (500 MHz, DMSO) δ 10.83 (s, 1H), 8.32 (d, J=8.3 Hz, 1H), 7.27 (d, J=5.4 Hz, 1H), 7.24 (d, J=5.4 Hz, 1H), 4.76-4.68 (m, 1H), 2.96 (tt, I=8.4, 5.2 Hz, 1H), 2.78 (ddd, J=17.4, 13.3, 5.5 Hz, 1H), 2.57-2.51 (m, 1H), 2.11 (qd, J=13.0, 4.5 Hz, 1H), 1.98 (dddd, J=11.0, 8.3, 5.4, 2.9 Hz, 1H), 1.10 (ddd, J=8.4, 6.0, 4.0 Hz, 2H), 0.68-0.63 (m, 2H).

LCMS (m/z [M+H]+): 279.1

### Example 63: Synthesis of N-(2,6-dioxopiperidin-3-yl)-5-methoxyoxazole-4-carboxamide (67)

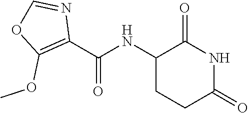 [SMILES: COc1ocnc1C(=O)NC1CCC(=O)NC1=O]

This compound was synthesized using the general procedure shown in Reaction Scheme 1 and Synthetic Conditions C, above, (45% yield) using 5-methoxy-1,3-oxazole-4-carboxylic acid (7 mg) as a starting material.

¹H NMR (500 MHz, DMSO) δ 10.80 (s, 1H), 8.11 (d, J=8.4 Hz, 1H), 8.01 (s, 1H), 4.67 (ddd, J=12.5, 8.4, 5.3 Hz, 1H), 4.12 (s, 3H), 2.81-2.73 (m, 1H), 2.58 (s, 1H), 2.20-2.09 (m, 1H), 1.97-1.89 (m, 1H).

LCMS (m/z [M+H]⁺): 254.0

### Example 64: Synthesis of N-(2,6-dioxopiperidin-3-yl)-5-ethoxy-2-methyloxazole-4-carboxamide (68)

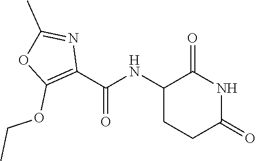 [SMILES: CCOc1oc(C)nc1C(=O)NC1CCC(=O)NC1=O]

This compound was synthesized using the general procedure shown in Reaction Scheme 1 and Synthetic Conditions D, above, (86% yield) using 5-ethoxy-2-methyloxazole-4-carboxylic acid (30 mg) as a starting material.

1H NMR (500 MHz, DMSO) δ 10.79 (s, 1H), 8.04 (d, J=8.3 Hz, 1H), 4.69-4.60 (m, 1H), 4.44 (q, J=7.1 Hz, 2H), 2.75 (ddd, J=17.4, 13.7, 5.5 Hz, 1H), 2.48 (d, J=2.8 Hz, 1H), 2.33 (s, 3H), 2.13 (qd, J=12.9, 4.5 Hz, 1H), 1.93 (dddd, J=10.8, 7.9, 5.4, 2.5 Hz, 1H), 1.32 (t, J=7.1 Hz, 3H).

LCMS (m/z [M+H]⁺): 282.1

### Example 65: Synthesis of N-(2,6-dioxopiperidin-3-yl)-5-methoxy-2-phenyloxazole-4-carboxamide (69)

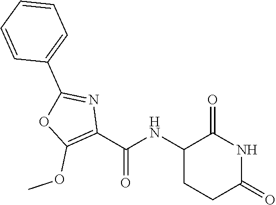 [SMILES: COc1oc(-c2ccccc2)nc1C(=O)NC1CCC(=O)NC1=O]

This compound was synthesized using the general procedure shown in Reaction Scheme 1 and Synthetic Conditions C, above (39% yield), and 5-methoxy-2-phenyloxazole-4-carboxylic acid (9.4 mg) as a starting material.

¹H NMR (500 MHz, DMSO) δ 10.83 (s, 1H), 8.18 (d, J=8.4 Hz, 1H), 7.97-7.92 (m, 2H), 7.58-7.50 (m, 3H), 4.73 (ddd, J=12.5, 8.4, 5.3 Hz, 1H), 4.23 (s, 3H), 2.84-2.73 (m, 1H), 2.59-2.52 (m, 1H), 2.18 (qd, J=12.9, 4.4 Hz, 1H), 1.97 (dtd, J=12.9, 5.4, 2.6 Hz, 1H).

LCMS (m/z [M+H]⁺): 330.0

### Example 66: Synthesis of N-(2,6-dioxopiperidin-3-yl)-5-morpholino-2-phenyloxazole-4-carboxamide (70)

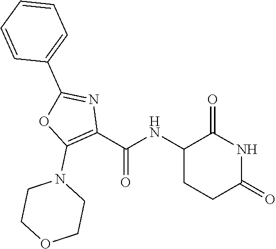 [SMILES: O=C1CCC(NC(=O)c2nc(-c3ccccc3)oc2N2CCOCC2)C(=O)N1]

This compound was synthesized using the general procedure shown in Reaction Scheme 1 and Synthetic Conditions D, above, (27% yield) using 5-(morpholin-4-yl)-2-phenyl-1,3-oxazole-4-carboxylic acid (8 mg) as a starting material.

1H NMR (500 MHz, DMSO) δ 10.82 (s, 1H), 8.15 (d, J=8.2 Hz, 1H), 7.94-7.88 (m, 2H), 7.56-7.45 (m, 3H), 4.67 (ddd, J=12.8, 8.1, 5.3 Hz, 1H), 3.73 (s, 8H), 2.76 (ddd, J=17.2, 13.6, 5.4 Hz, 1H), 2.58 (d, J=4.9 Hz, 1H), 2.18 (qd, J=12.9, 4.4 Hz, 1H), 2.03-1.93 (m, 1H).

LCMS (m/z [M+H]⁺): 385.1

### Example 67: Synthesis of tert-butyl (5-((2,6-dioxopiperidin-3-yl)carbamoyl)thiazol-2-yl)carbamate (71)

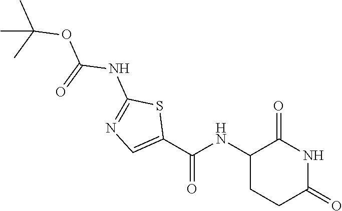 [SMILES: CC(C)(C)OC(=O)Nc1ncc(C(=O)NC2CCC(=O)NC2=O)s1]

This compound was synthesized using the general procedure shown in Reaction Scheme 1 and Synthetic Conditions C, above, (31% yield) using 2-((tert-butoxycarbonyl)amino)thiazole-5-carboxylic acid (20 mg) as a starting material.

1H NMR (500 MHz, DMSO) δ 11.73 (s, 1H), 10.86 (s, 1H), 8.70 (d, J=8.4 Hz, 1H), 8.00 (s, 1H), 4.78-4.66 (m, 1H), 2.78 (ddd, J=17.5, 13.2, 5.6 Hz, 1H), 2.52-2.51 (m, 1H), 2.07 (qd, J=12.9, 4.5 Hz, 1H), 1.97 (dddd, J=11.3, 8.4, 5.5, 2.9 Hz, 1H), 1.50 (s, 9H).

### Example 68: Synthesis of N-(2,6-dioxopiperidin-3-yl)-2-(trifluoromethyl)thiazole-5-carboxamide (72)

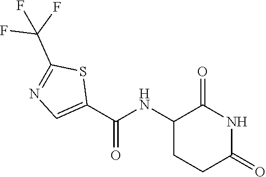 [SMILES: O=C1CCC(NC(=O)c2cnc(C(F)(F)F)s2)C(=O)N1]

This compound was synthesized using the general procedure shown in Reaction Scheme 1 and Synthetic Conditions C, above (29% yield) using 2-(trifluoromethyl)thiazole-5-carboxylic acid (20 mg) as a starting material.

¹H NMR (500 MHz, DMSO) δ 10.87 (s, 1H), 7.89 (d, J=7.4 Hz, 1H), 7.75 (d, J=5.5 Hz, 1H), 7.14 (d, J=5.5 Hz, 1H), 4.78-4.63 (m, 1H), 3.98 (s, 3H), 3.31 (s, 3H), 2.77 (ddd, J=17.4, 13.1, 6.3 Hz, 1H), 2.54-2.51 (m, 1H), 2.17-2.04 (m, 2H).

LCMS (m/z [M−H]⁻): 305.8

### Example 69: Synthesis of 2-(tert-butyl)-N-(2,6-dioxopiperidin-3-yl)thiazole-5-carboxamide (73)

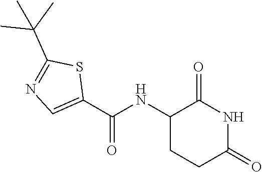 [SMILES: CC(C)(C)c1ncc(C(=O)NC2CCC(=O)NC2=O)s1]

This compound was synthesized using the general procedure shown in Reaction Scheme 1 and Synthetic Conditions C, above (83% yield) using 2-(tert-butyl)thiazole-5-carboxylic acid (20 mg) as a starting material.

¹H NMR (500 MHz, DMSO) δ 10.88 (s, 1H), 8.88 (d, J=8.4 Hz, 1H), 8.26 (s, 1H), 4.73 (ddd, J=13.4, 8.3, 5.4 Hz, 1H), 2.79 (ddd, J=17.5, 13.3, 5.6 Hz, 1H), 2.58-2.51 (m, 1H), 2.15-2.03 (m, 1H), 1.98 (ddd, J=12.9, 5.4, 2.8 Hz, 1H), 1.39 (s, 9H).

LCMS (m/z [M+H]⁺): 295.9

### Example 70: Synthesis of benzyl ((5-((2,6-dioxopiperidin-3-yl)carbamoyl)thiazol-2-yl)methyl)carbamate (74)

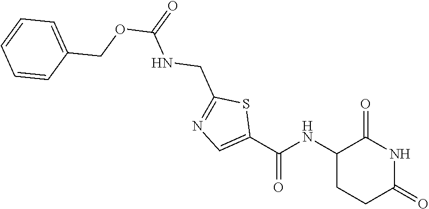 [SMILES: O=C1CCC(NC(=O)c2cnc(CNC(=O)OCc3ccccc3)s2)C(=O)N1]

This compound was synthesized using the general procedure shown in Reaction Scheme 1 and Synthetic Conditions C, above (68% yield) using 2-((((benzyloxy)carbonyl)amino)methyl)thiazole-5-carboxylic acid (20 mg) as a starting material.

¹H NMR (500 MHz, DMSO) δ 10.88 (s, 1H), 8.91 (d, J=8.3 Hz, 1H), 8.28 (s, 1H), 8.25 (t, J=5.9 Hz, 1H), 7.38 (d, J=4.1 Hz, 3H), 7.33 (dd, J=8.8, 4.5 Hz, 1H), 5.09 (s, 2H), 4.78-4.69 (m, 1H), 4.48 (d, J=6.1 Hz, 2H), 2.79 (ddd, J=18.6, 13.3, 5.6 Hz, 1H), 2.52 (d, J=1.9 Hz, 1H), 2.09 (qd, J=12.9, 4.4 Hz, 1H), 1.99 (tdd, J=8.2, 5.4, 2.8 Hz, 1H).

LCMS (m/z [M+H]⁺): 403.1

### Example 71: Synthesis of N-(2,6-dioxopiperidin-3-yl)-2-methoxythiazole-5-carboxamide (75)

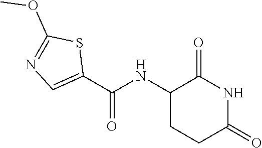 [SMILES: COc1ncc(C(=O)NC2CCC(=O)NC2=O)s1]

This compound was synthesized using the general procedure shown in Reaction Scheme 1 and Synthetic Conditions C, above (16% yield) using 2-methoxythiazole-5-carboxylic acid (20 mg) as a starting material.

¹H NMR (500 MHz, DMSO) δ 10.87 (s, 1H), 8.79 (d, J=8.3 Hz, 1H), 7.87 (s, 1H), 4.71 (ddd, J=12.6, 8.3, 5.4 Hz, 1H), 4.06 (s, 3H), 2.78 (ddd, J=17.5, 13.2, 5.6 Hz, 1H), 2.53-2.51 (m, 1H), 2.07 (qd, J=12.9, 4.5 Hz, 1H), 1.97 (dddd, J=11.0, 8.3, 5.5, 2.8 Hz, 1H).

LCMS (m/z [M+H]⁺): 269.9

### Example 72: Synthesis of N-(2,6-dioxopiperidin-3-yl)-2-(methylthio)thiazole-5-carboxamide (76)

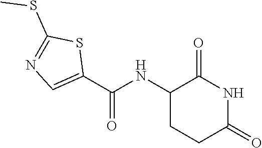 [SMILES: CSc1ncc(C(=O)NC2CCC(=O)NC2=O)s1]

This compound was synthesized using the general procedure shown in Reaction Scheme 1 and Synthetic Conditions C, above (43% yield) using 2-(methylthio)thiazole-5-carboxylic acid (20 mg) as a starting material.

¹H NMR (500 MHz, DMSO) δ 10.88 (s, 1H), 8.90 (d, J=8.3 Hz, 1H), 8.26 (s, 1H), 4.73 (ddd, J=12.6, 8.3, 5.4 Hz, 1H), 2.79 (ddd, J=17.5, 13.2, 5.6 Hz, 1H), 2.72 (s, 3H), 2.52 (dd, J=4.0, 2.2 Hz, 1H), 2.13-2.03 (m, 1H), 1.99 (dddd, J=11.1, 8.3, 5.5, 2.8 Hz, 1H).

LCMS (m/z [M+H]⁺): 285.9

### Example 73: Synthesis of tert-butyl ((4-((2,6-dioxopiperidin-3-yl)carbamoyl)thiazol-2-yl)methyl)carbamate (77)

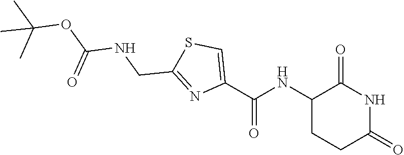 [SMILES: CC(C)(C)OC(=O)NCc1nc(C(=O)NC2CCC(=O)NC2=O)cs1]

This compound was synthesized using the general procedure shown in Reaction Scheme 1 and Synthetic Conditions C, above (39% yield) using 2-(((tert-butoxycarbonyl)amino)methyl)thiazole-4-carboxylic acid (50 mg) as a starting material.

¹H NMR (500 MHz, DMSO) δ 10.83 (s, 1H), 8.59 (d, J=8.5 Hz, 1H), 8.21 (s, 1H), 7.86 (t, J=6.1 Hz, 1H), 4.76 (ddd, J=12.6, 8.5, 5.3 Hz, 1H), 4.43 (d, J=6.1 Hz, 2H), 2.79 (ddd, J=17.3, 13.7, 5.5 Hz, 1H), 2.59-2.53 (m, 1H), 2.19 (qd, J=13.0, 4.5 Hz, 1H), 1.97 (dtd, J=12.9, 5.4, 2.6 Hz, 1H), 1.42 (s, 9H).

LCMS (m/z [M-Boc+H]+): 268.9

### Example 74: Synthesis of N-(2,6-dioxopiperidin-3-yl)-5-(methylamino)thiazole-4-carboxamide (78)

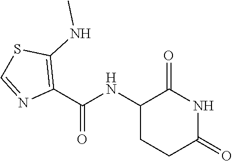 [SMILES: CNc1scnc1C(=O)NC1CCC(=O)NC1=O]

To a solution of 5-(methylamino)thiazole-4-carboxylic acid (1 eq.) in isopropanol and water were added 3-aminopiperidine-2,6-dione (hydrochloride salt, 3.0 eq.), N-methylmorpholine (3 eq.) and DMTMM (1.5 eq.). The reaction mixture was stirred overnight at RT, concentrated under reduced pressure and purified by HPLC to give N-(2,6-dioxopiperidin-3-yl)-5-(methylamino)thiazole-4-carboxamide (13% yield).

¹H NMR (500 MHz, DMSO) δ 10.78 (s, 1H), 8.11 (d, J=0.9 Hz, 1H), 8.04 (d, J=8.3 Hz, 1H), 7.60 (q, J=5.0 Hz, 1H), 4.64 (ddd, J=12.4, 8.3, 5.3 Hz, 1H), 2.92 (d, J=5.0 Hz, 3H), 2.76 (ddd, J=17.2, 13.7, 5.5 Hz, 1H), 2.59-2.51 (m, 1H), 2.23-2.12 (m, 1H), 1.96 (dtd, J=13.0, 5.4, 2.6 Hz, 1H).

LCMS (m/z [M+H]⁺): 268.7

### Example 75: Synthesis of N-(2,6-dioxopiperidin-3-yl)-5-methoxythiazole-4-carboxamide (79)

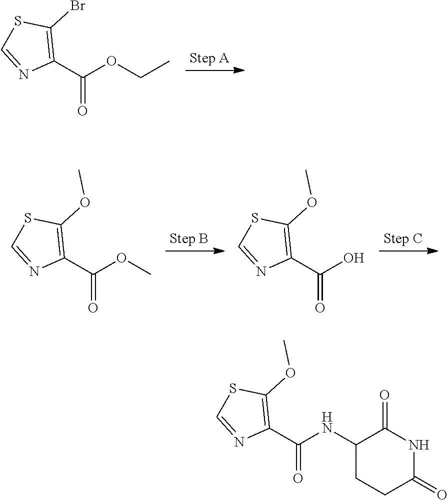

Step A: To a stirred solution of ethyl 5-bromothiazole-4-carboxylate (2.0 g, 8.475 mmol, 1 eq) in methanol (24 mL) was added NaOMe (25% in MeOH) (3.8 ml, 16.95 mmol, 2 eq). The reaction mixture was refluxed for 2 h, cooled to RT and quenched by saturated ammonium chloride solution (10 mL). The mixture was concentrated under reduced pressure and purified by flash column chromatography to give methyl 5-methoxythiazole-4-carboxylate (27% yield).

Step B: To a stirring solution of methyl 5-methoxythiazole-4-carboxylate (100 mg, 0.578 mmol, 1 eq) in a solution of THF, MeOH, H₂O (4:2:1) (7 mL) was added LiOH, H₂O (73 mg, 1.734 mmol, 3 eq). The reaction mixture was stirred at RT for 16 h, evaporated, redissolved in water and washed with ethyl acetate. The aqueous layer was acidified by 0.5 M HCl, extracted with 10% MeOH in DCM, dried over Na₂SO₄, concentrated under reduced pressure and purified by flash column chromatography to give 5-methoxythiazole-4-carboxylic acid (32% yield).

Step C: N-(2,6-dioxopiperidin-3-yl)-5-methoxythiazole-4-carboxamide was synthesized using the general procedure shown in Reaction Scheme 1 and Synthetic Conditions C, above, (9% yield) using 5-methoxythiazole-4-carboxylic acid (20 mg) as a starting material.

¹H NMR (500 MHz, DMSO) δ 10.81 (s, 1H), 8.51 (s, 1H), 8.23 (d, J=8.2 Hz, 1H), 4.67 (ddd, J=12.5, 8.2, 5.3 Hz, 1H), 4.04 (s, 3H), 2.83-2.73 (m, 1H), 2.52 (dt, J=3.9, 2.5 Hz, 1H), 2.19-2.09 (m, 1H), 1.97 (dtd, J=12.7, 5.5, 2.6 Hz, 1H).

LCMS (m/z [M+H]⁺): 269.8

### Example 76: Synthesis of 2-amino-N-(2,6-dioxopiperidin-3-yl)-5-methoxythiazole-4-carboxamide (80)

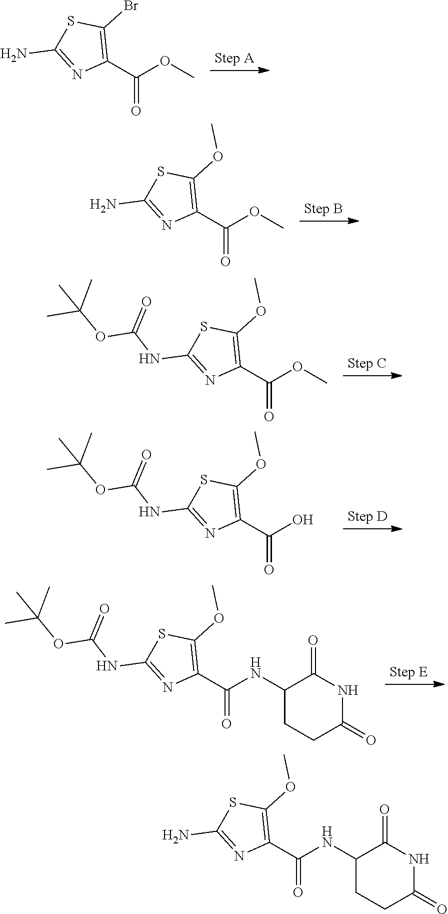

Step A: To a stirring solution of methyl 2-amino-5-bromothiazole-4-carboxylate (1 g, 4.255 mmol, 1 eq) in methanol (30 mL) was added NaOMe (25% in MeOH) (2.3 ml, 10.638 mmol, 2.5 eq). The reaction mixture was refluxed for 1.5 h, cooled to RT and quenched by saturated ammonium chloride solution (10 mL). The mixture was concentrated under reduced pressure and purified by flash column chromatography to give methyl 2-amino-5-methoxythiazole-4-carboxylate (50% yield).

Step B: Methyl 2-amino-5-methoxythiazole-4-carboxylate (400 mg, 2.128 mmol. 1 eq.) was dissolved in DCM then were added triethylamine (0.532 mmol, 2 eq.) and Boc₂O (0.532 mmol, 2 eq). The reaction mixture was stirred at RT for 18 h, diluted with DCM and washed successively with water and brine, dried over Na₂SO₄, concentrated under reduced pressure and purified by flash column chromatography to give methyl 2-((tert-butoxycarbonyl)amino)-5-methoxythiazole-4-carboxylate (49% yield).

Step C: To a stirring solution of methyl 2-((tert-butoxycarbonyl)amino)-5-methoxythiazole-4-carboxylate (300 mg, 1.042 mmol, 1 eq) in THF:MeOH:H₂O 3:2:1 (12 mL) was added LiOH.H₂O (131 mg, 3.125 mmol, 3 eq). The reaction mixture was stirred at RT for 16 h, evaporated, redissolved in water and washed with ethyl acetate. The aqueous layer was acidified by 0.5 M HCl, extracted with 10% MeOH in DCM, dried over Na₂SO₄, concentrated under reduced pressure and triturated with ether and pentane to give 2-((tert-butoxycarbonyl)amino)-5-methoxythiazole-4-carboxylic acid (49% yield).

Step D: Tert-butyl (4-((2,6-dioxopiperidin-3-yl)carbamoyl)-5-methoxythiazol-2-yl)carbamate was synthesized using the general procedure shown in Reaction Scheme 1 and Synthetic Conditions C, above (50% yield), and 2-((tert-butoxycarbonyl)amino)-5-methoxythiazole-4-carboxylic acid (20 mg) as a starting material.

Step E: To a solution of tert-butyl (4-((2,6-dioxopiperidin-3-yl)carbamoyl)-5-methoxythiazol-2-yl)carbamate (19.6 mg, 0.051 mmol, 1 eq.) in water (3 mL) and dioxane (3 mL) was added 36% HCl (1.5 mL). The reaction was stirred at RT for 3 h and concentrated under reduced pressure to give 2-amino-N-(2,6-dioxopiperidin-3-yl)-5-methoxythiazole-4-carboxamide hydrochloride (100% yield).

¹H NMR (500 MHz, DMSO) δ 10.84 (s, 1H), 7.75 (d, J=7.8 Hz, 1H), 7.41-6.65 (m, 2H), 4.63 (ddd, J=12.0, 7.8, 5.8 Hz, 1H), 3.89 (s, 3H), 2.75 (ddd, J=17.3, 13.1, 6.2 Hz, 1H), 2.60-2.52 (m, 1H), 2.11-1.98 (m, 2H).

LCMS (m/z [M+H]⁺): 285.0

### Example 77: Synthesis of 4-((2,6-dioxopiperidin-3-yl)carbamoyl)-5-methoxythiazole-2-carboxylic acid (81)

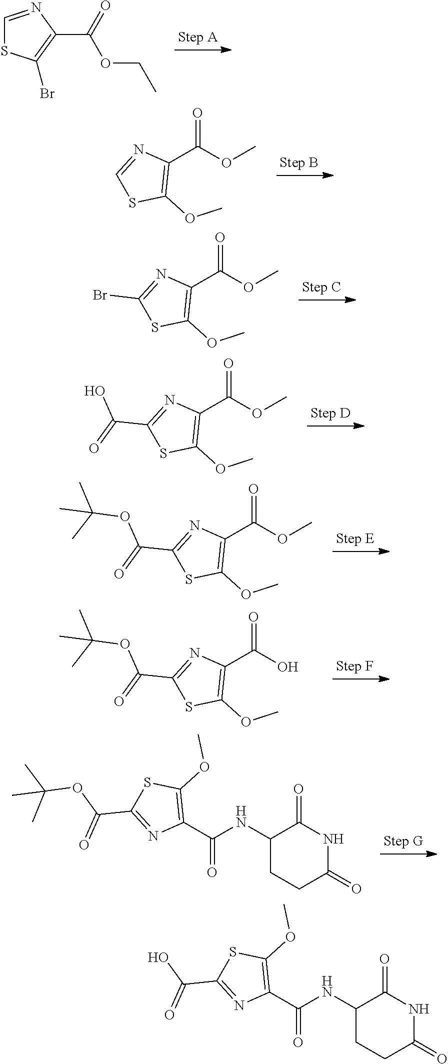

Step A: To a stirred solution of ethyl 5-bromothiazole-4-carboxylate (2.0 g, 8.475 mmol) in methanol (24 mL) was added NaOMe (25% in MeOH) (3.8 ml, 16.95 mmol, 2 eq). Reaction mixture was then refluxed for 2 h, cooled to RT and quenched by ammonium chloride solution. The product was extracted with ethyl acetate, dried over Na₂SO₄, concentrated under reduced pressure and purified by flash column chromatography to give methyl 5-methoxythiazole-4-carboxylate (27% yield).

Step B: To a solution of methyl 5-methoxythiazole-4-carboxylate (70 mg, 0.405 mmol, 1 eq) in THF (5 mL) was added N-bromosuccinimide (288 mg, 1.618 mmol, 4 eq) and the reaction mixture was stirred at RT for 24 h. The reaction mixture was diluted with ethyl acetate, washed with water and brine, dried over Na₂SO₄, concentrated under reduced pressure and purified by flash column chromatography to give methyl 2-bromo-5-methoxythiazole-4-carboxylate (73% yield).

Step C: To a solution of methyl 2-bromo-5-methoxythiazole-4-carboxylate (1.0 g, 3.968 mmol, 1 eq) in THF (30 mL) and water (15 mL) was added triethylamine (2.701 ml, 19.841 mmol, 5 eq) and the solution was purged with argon for 10 min. Xantphos (0.115 g, 0.198 mmol, 0.05 eq) and Pd(OAc)₂ (44 mg, 0.198 mmol, 0.05 eq) were added and the reaction mixture was stirred at 60° C. under CO (50 psi) for 16 h. The reaction mixture was cooled to RT, diluted with water and washed with ethyl acetate. The aqueous layer was acidified by 2M HCl solution, extracted with 15% MeOH in DCM, dried over Na₂SO₄ and concentrated under reduced pressure to give 5-methoxy-4-(methoxycarbonyl)thiazole-2-carboxylic acid (30% yield).

Step D: To a solution of 5-methoxy-4-(methoxycarbonyl)thiazole-2-carboxylic acid (300 mg, 1.382 mmol, 1 eq) in tert-butanol (15 mL) was added 2-tert-butyl-1,3-diisopropylisourea (829 mg, 4.147 mmol, 3 eq) and the reaction mixture was stirred at RT for 16 h, diluted with ethyl acetate and washed by water, dried over Na₂SO₄, concentrated under reduced pressure and purified by flash column chromatography to give 2-tert-butyl 4-methyl 5-methoxythiazole-2,4-dicarboxylate (31% yield).

Step E: To a solution of 2-tert-butyl 4-methyl 5-methoxythiazole-2,4-dicarboxylate (220 mg, 0.806 mmol, 1 eq) in DCE (5 mL) was added trimethyltin hydroxide (728 mg, 4.029 mmol, 5 eq). Reaction mixture was stirred at 90° C. for 6 h, filtered, the filtrate was concentrated under reduced pressure and purified by HPLC to give 2-(tert-butoxycarbonyl)-5-methoxythiazole-4-carboxylic acid (11% yield).

Step F: tert-butyl 4-((2,6-dioxopiperidin-3-yl)carbamoyl)-5-methoxythiazole-2-carboxylate was synthesized using the general procedure shown in Reaction Scheme 1 and Synthetic Conditions C, above, (39% yield) using 2-(tert-butoxycarbonyl)-5-methoxythiazole-4-carboxylic acid (16.5 mg) as a starting material.

¹H NMR (500 MHz, DMSO) δ 10.83 (s, 1H), 8.24 (d, J=8.2 Hz, 1H), 4.77-4.65 (m, 1H), 4.14 (s, 3H), 2.77 (ddd, J=17.2, 13.8, 5.5 Hz, 1H), 2.60-2.53 (m, OH), 2.17 (qd, J=12.9, 4.5 Hz, 1H), 1.96 (dtd, J=12.9, 5.5, 2.6 Hz, 1H), 1.56 (s, 9H).

Step G: tert-butyl 4-((2,6-dioxopiperidin-3-yl)carbamoyl)-5-methoxythiazole-2-carboxylate (6 mg, 0.016 mmol) was dissolved in DCM (0.5 mL) and trifluoroacetic acid (0.092 mL) was added. The Reaction was stirred at RT for 2 h and concentrated under reduced pressure to give 4-((2,6-dioxopiperidin-3-yl)carbamoyl)-5-methoxythiazole-2-carboxylic acid (71% yield).

¹H NMR (500 MHz, DMSO) δ 14.03 (s, 1H), 10.82 (s, 1H), 8.27 (d, J=8.3 Hz, 1H), 4.71 (ddd, J=12.5, 8.3, 5.4 Hz, 1H), 4.14 (s, 3H), 2.77 (ddd, J=17.1, 13.7, 5.5 Hz, 1H), 2.58 (s, 1H), 2.16 (qd, J=12.9, 4.5 Hz, 1H), 1.96 (dtd, J=12.9, 5.4, 2.6 Hz, 1H).

LCMS (m/z [M+H]⁺): 314.0

### Example 78: Synthesis of 5-Cyclopropyl-N-(2,6-dioxopiperidin-3-yl)thiazole-4-carboxamide (82)

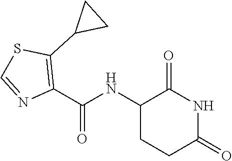 [SMILES: O=C1CCC(NC(=O)c2ncsc2C2CC2)C(=O)N1]

This compound was synthesized using the general procedure shown in Reaction Scheme 1 and Synthetic Conditions C, above, (67% yield) using 5-cyclopropyl-1,3-thiazole-4-carboxylic (20 mg) as a starting material.

¹H NMR (500 MHz, DMSO) δ 10.83 (s, 1H), 8.86 (d, J=0.7 Hz, 1H), 8.59 (d, J=8.3 Hz, 1H), 4.73 (ddd, J=12.6, 8.3, 5.3 Hz, 1H), 3.35-3.31 (m, 1H), 2.78 (ddd, J=17.4, 13.7, 5.6 Hz, 1H), 2.59-2.51 (m, 1H), 2.25-2.15 (m, 1H), 2.03-1.97 (m, 1H), 1.28-1.20 (m, 2H), 0.68 (pd, J=4.4, 1.9 Hz, 2H).

LCMS (m/z [M+H]⁺): 280.1

### Example 79: Synthesis of N-(2,6-dioxopiperidin-3-yl)-4,5-dimethyl-2-pivalamidothiophene-3-carboxamide (83)

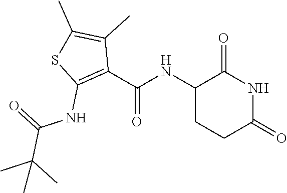 [SMILES: Cc1sc(NC(=O)C(C)(C)C)c(C(=O)NC2CCC(=O)NC2=O)c1C]

This compound was synthesized using the general procedure shown in Reaction Scheme 1 and Synthetic Conditions C, above (4% yield), and 4,5-dimethyl-2-pivalamidothiophene-3-carboxylic acid (22.5 mg) as a starting material.

¹H NMR (500 MHz, DMSO) δ 11.17 (s, 1H), 10.91 (s, 1H), 7.93 (d, J=8.1 Hz, 1H), 4.80 (ddd, J=13.0, 8.1, 5.4 Hz, 1H), 2.81 (ddd, J=18.4, 13.5, 5.6 Hz, 1H), 2.59-2.51 (m, 1H), 2.24 (s, 3H), 2.22 (s, 3H), 2.14 (qd, J=13.0, 4.5 Hz, 1H), 2.07-2.01 (m, 1H), 1.21 (s, 9H).

LCMS (m/z [M+H]⁺): 366.15

### Example 80: Synthesis of 2-benzamido-N-(2,6-dioxopiperidin-3-yl)-4,5-dimethylthiophene-3-carboxamide (87)

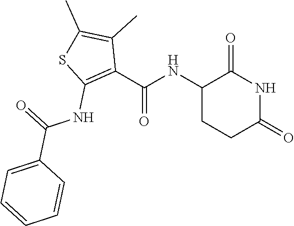 [SMILES: Cc1sc(NC(=O)c2ccccc2)c(C(=O)NC2CCC(=O)NC2=O)c1C]

This compound was synthesized using the general procedure shown in Reaction Scheme 1 and Synthetic Conditions C, above, (27% yield) using 2-benzamido-4,5-dimethylthiophene-3-carboxylic acid (20 mg) as a starting material.

¹H NMR (500 MHz, DMSO) δ 11.77 (s, 1H), 10.94 (s, 1H), 8.11 (d, J=8.2 Hz, 1H), 7.90 (dt, J=7.1, 1.4 Hz, 2H), 7.68-7.61 (m, 1H), 7.58 (dd, J=8.4, 6.9 Hz, 2H), 4.90-4.80 (m, 1H), 2.82 (ddd, J=18.5, 13.3, 5.7 Hz, 1H), 2.58 (d, J=3.5 Hz, 1H), 2.29 (s, 3H), 2.24 (s, 3H), 2.21-2.04 (m, 2H).

LCMS (m/z [M−1-1]1: 386.0

### Example 81: Synthesis of N-(2,6-dioxopiperidin-3-yl)-2-(4-methoxybenzamido)-4,5,6,7-tetrahydrobenzo[b]thiophene-3-carboxamide (89)

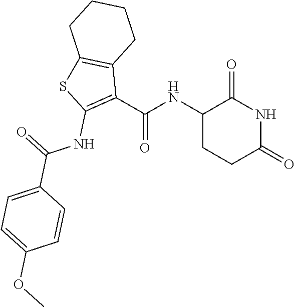 [SMILES: COc1ccc(C(=O)Nc2sc3c(c2C(=O)NC2CCC(=O)NC2=O)CCCC3)cc1]

This compound was synthesized using the general procedure shown in Reaction Scheme 1 and Synthetic Conditions C, above, (3.9% yield) 2-(4-methoxybenzamido)-4,5,6,7-tetrahydrobenzo[b]thiophene-3-carboxylic acid (30 mg) as a starting material.

¹H NMR (500 MHz, DMSO, 300 K): δ 12.28 (s, 1H), 10.92 (s, 1H), 7.85 (d, J=8.8 Hz, 2H), 7.72 (d, J=8.0 Hz, 1H), 7.14 (d, J=8.6 Hz, 2H), 4.91-4.80 (m, 1H), 3.85 (s, 3H), 2.87-2.79 (m, 1H), 2.77 (d, J=5.5 Hz, 2H), 2.67 (s, 2H), 2.59-2.53 (m, 1H), 2.24-2.02 (m, 2H), 1.77 (s, 4H).

LCMS (m/z [M+H]⁺): 441.9

### Example 82: Synthesis of N-(2,6-dioxopiperidin-3-yl)-2-(2-morpholinoacetamido)-4,5,6,7-tetrahydrobenzo[b]thiophene-3-carboxamide (90)

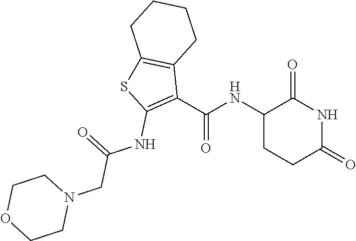 [SMILES: O=C1CCC(NC(=O)c2c(NC(=O)CN3CCOCC3)sc3c2CCCC3)C(=O)N1]

This compound was synthesized using the general procedure shown in Reaction Scheme 1 and Synthetic Conditions C, above, (25% yield) using triethylamine instead of DIPEA and 2-(2-morpholinoacetamido)-4,5,6,7-tetrahydrobenzo[b]thiophene-3-carboxylic acid (18 mg) as a starting material.

¹H NMR (500 MHz, DMSO) δ 11.84 (s, 1H), 10.86 (s, 1H), 7.69 (d, J=8.2 Hz, 1H), 4.77 (ddd, 0.1=13.0, 8.1, 5.4 Hz, 1H), 3.71-3.59 (m, 4H), 3.19 (s, 2H), 2.83 (ddd, J=17.4, 13.6, 5.7 Hz, 1H), 2.78-2.67 (m, 2H), 2.67-2.61 (m, 2H), 2.58-2.53 (m, 1H), 2.48 (d, J=4.2 Hz, 2H), 2.14 (qd, J=12.9, 4.4 Hz, 1H), 2.03 (ddt, J=13.6, 6.8, 3.3 Hz, 1H), 1.82-1.67 (m, 4H).

LCMS (m/z [M+H]⁺): 435.1

### Example 83: Synthesis of N-(2,6-dioxopiperidin-3-yl)-2,4-dimethylthieno[3,4-b]pyridine-7-carboxamide (92)

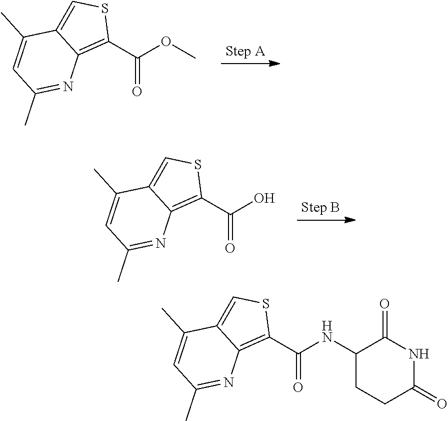

Step A: To a solution of methyl 2,4-dimethylthieno[3,4-b]pyridine-7-carboxylate (25.0 mg, 0.113 mmol, 1.000 eq) in a mixture of H₂O (1.0 mL), THF (1.0 mL) and MeOH (1.0 mL) was added 1M LiOH (2.0 mL, 2.000 mmol, 17.7 eq) and the reaction was stirred at RT for 24 h and neutralized with 1M HCl. After concentration under reduced pressure 2,4-dimethylthieno[3,4-b]pyridine-7-carboxylic acid was used in the next step without further purification.

Step B: N-(2,6-dioxopiperidin-3-yl)-2,4-dimethylthieno[3,4-b]pyridine-7-carboxamide was synthesized using the general procedure shown in Reaction Scheme 1 and Synthetic Conditions C, above (53% yield), and 2,4-dimethylthieno[3,4-b]pyridine-7-carboxylic acid (23.4 mg) as a starting material.

¹H NMR (500 MHz, DMSO) δ 10.95 (s, 1H), 10.08 (d, J=6.8 Hz, 1H), 8.52 (s, 1H), 7.02 (t, J=1.1 Hz, 1H), 4.84 (ddd, J=12.3, 6.8, 5.3 Hz, 1H), 2.82 (ddd, J=17.4, 13.6, 5.5 Hz, 1H), 2.61 (s, 3H), 2.59-2.53 (m, 4H), 2.32 (dtd, J=12.9, 5.4, 2.4 Hz, 1H), 2.12 (qd, J=12.9, 4.4 Hz, 1H).

LCMS (m/z [M+H]⁺): 317.9

### Example 84: Synthesis of 4-chloro-N-(2,6-dioxopiperidin-3-yl)-2-(trifluoromethyl)thieno[3,4-b]pyridine-7-carboxamide (93)

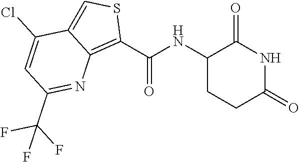 [SMILES: O=C1CCC(NC(=O)c2scc3c(Cl)cc(C(F)(F)F)nc23)C(=O)N1]

4-chloro-2-(trifluoromethyl)thieno[3,4-b]pyridine-7-carboxylic acid (1 eq.) and DMF (1 μL) were dissolved in DCM (3 mL) and then oxalyl chloride (0.018 mL, 0.213 mmol, 3.000 eq) was added. The reaction mixture was stirred at RT for 2 h and concentrated under reduced pressure. The material was dissolved in DMF (2 mL), 3-aminopiperidine-2,6-dione hydrochloride (2 eq.) and DIPEA (2 eq.) were added and the reaction mixture was stirred at RT for 72 h and purified by preparative HPLC to give 4-chloro-N-(2,6-dioxopiperidin-3-yl)-2-(trifluoromethyl)thieno[3,4-b]pyridine-7-carboxamide (6% yield).

¹H NMR (500 MHz, DMSO): δ 11.03 (s, 1H), 9.30 (d, J=6.4 Hz, 1H), 8.90 (s, 1H), 8.03 (s, 1H), 4.93-4.84 (m, 1H), 2.82 (ddd, J=18.8, 13.6, 5.5 Hz, 1H), 2.61-2.54 (m, 1H), 2.40 (ddd, J=7.2, 5.3, 2.7 Hz, 1H), 2.11-2.02 (m, 1H).

LCMS (m/z [M+H]⁺): 391.8

### Example 85: Synthesis of N-(2,6-dioxopiperidin-3-yl)-4-hydroxy-2-(trifluoromethyl)thieno[3,4-b]pyridine-7-carboxamide (94)

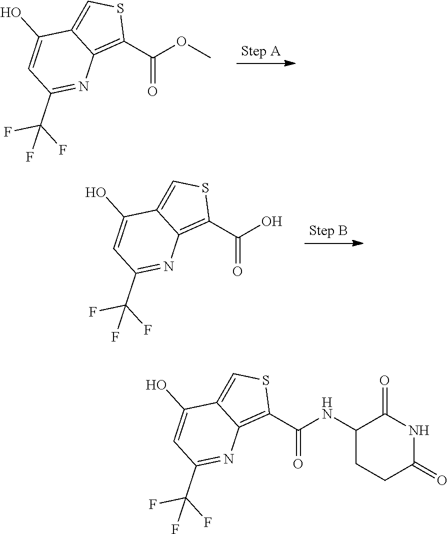 [SMILES: COC(=O)c1scc2c(O)cc(C(F)(F)F)nc12 ; O=C(O)c1scc2c(O)cc(C(F)(F)F)nc12 ; O=C1CCC(NC(=O)c2scc3c(O)cc(C(F)(F)F)nc23)C(=O)N1]

Step A: To a solution of methyl 4-hydroxy-2-(trifluoromethyl)thieno[3,4-b]pyridine-7-carboxylate (30.0 mg, 0.108 mmol, 1.000 eq) in MeOH (2.0 mL) was added NaOH (216 mg, 5.411 mmol, 50 eq). The reaction was stirred at RT for 24 h and neutralized with 1M HCl. After concentration under reduced pressure 4-hydroxy-2-(trifluoromethyl)thieno[3,4-b]pyridine-7-carboxylic acid was used in the next step without further purification.

Step B: N-(2,6-dioxopiperidin-3-yl)-4-hydroxy-2-(trifluoromethyl)thieno[3,4-b]pyridine-7-carboxamide was synthesized using the general procedure shown in Reaction Scheme 1 and Synthetic Conditions C, above (24% yield), and 4-hydroxy-2-(trifluoromethyl)thieno[3,4-b]pyridine-7-carboxylic acid (28 mg) as a starting material.

¹H NMR (500 MHz, DMSO): δ 12.98 (s, 1H), 10.98 (s, 1H), 9.53 (d, J=6.6 Hz, 1H), 8.76 (s, 1H), 6.76 (s, 1H), 4.91-4.81 (m, 1H), 2.81 (ddd, J=18.6, 13.4, 5.4 Hz, 1H), 2.60-2.54 (m, 1H), 2.38-2.33 (m, 1H), 2.08-1.99 (m, 1H).

LCMS (m/z [M+H]⁺): 374.0

### Example 86: Synthesis of 7-bromo-N-(2,6-dioxopiperidin-3-yl)thieno[3,4-b]pyridine-5-carboxamide (96)

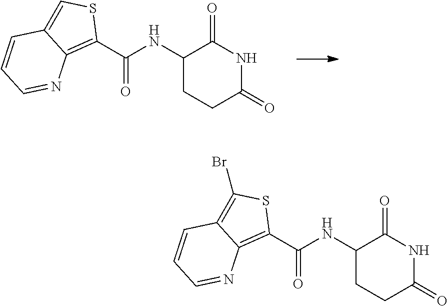 [SMILES: O=C1CCC(NC(=O)c2scc3cccnc23)C(=O)N1 ; O=C1CCC(NC(=O)c2sc(Br)c3cccnc23)C(=O)N1]

N-bromosuccinimide (96.8 mg, 0.544 mmol, 1.1 eq) was added to a suspension of N-(2,6-dioxopiperidin-3-yl)thieno[3,4-b]pyridine-7-carboxamide (143.0 mg, 0.494 mmol, 1.000 eq,) in DMF (4.9 mL) at ambient temperature. The reaction mixture was heated to 60° C. and stirred for 3 h. The obtained crude compound was purified by HPLC to give 5-bromo-N-(2,6-dioxopiperidin-3-yl)thieno[3,4-b]pyridine-7-carboxamide (15% yield).

¹H NMR (500 MHz, DMSO) δ 10.97 (s, 1H), 9.53 (d, J=7.4 Hz, 1H), 8.87 (dd, J=4.0, 1.5 Hz, 1H), 8.11 (dd, J=8.9, 1.5 Hz, 1H), 7.41 (dd, J=8.9, 4.0 Hz, 1H), 4.91 (ddd, J=12.8, 7.3, 5.6 Hz, 1H), 2.84 (ddd, J=17.5, 13.4, 5.7 Hz, 1H), 2.57 (ddd, J=17.4, 4.3, 2.3 Hz, 1H), 2.30-2.13 (m, 2H)

LCMS (m/z [M+H]⁺): 368.37

### Example 87: Synthesis of 4-chloro-N-(2,6-dioxopiperidin-3-yl)-1H-pyrrolo[2,3-b]pyridine-3-carboxamide (97)

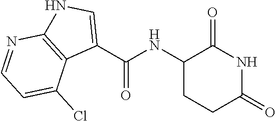 [SMILES: O=C1CCC(NC(=O)c2c[nH]c3nccc(Cl)c23)C(=O)N1]

This compound was synthesized using the general procedure shown in Reaction Scheme 1 and Synthetic Conditions C, above, (15% yield) using 4-chloro-1H-pyrrolo[2,3-b]pyridine-3-carboxylic acid (20 mg) as a starting material.

¹H NMR (500 MHz, DMSO) δ 12.40 (s, 1H), 10.82 (s, 1H), 8.38 (d, J=8.3 Hz, 1H), 8.22 (d, J=5.1 Hz, 1H), 7.96 (s, 1H), 7.25 (d, J=5.1 Hz, 1H), 4.79-4.72 (m, 1H), 2.79 (ddd, J=17.9, 9.7, 7.0 Hz, 1H), 2.59-2.52 (m, 1H), 2.12-2.01 (m, 2H).

LCMS (m/z [M+H]⁺): 306.9

### Example 88: Synthesis of N-(2,6-dioxopiperidin-3-yl)-1-methyl-1H-pyrrolo[2,3-b]pyridine-3-carboxamide (98)

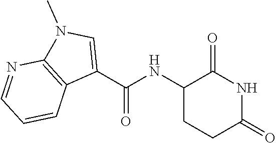 [SMILES: Cn1cc(C(=O)NC2CCC(=O)NC2=O)c2cccnc21]

This compound was synthesized using the general procedure shown in Reaction Scheme 1 and Synthetic Conditions C, above, (61% yield) using 1-methyl-1H-pyrrolo[2,3-b]pyridine-3-carboxylic acid (20 mg) as a starting material.

1H NMR (500 MHz, DMSO) δ 10.84 (s, 1H), 8.44 (dd, J=7.9, 1.6 Hz, 1H), 8.34 (dd, J=4.7, 1.6 Hz, 1H), 8.30 (d, J=8.3 Hz, 1H), 8.21 (s, 1H), 7.23 (dd, J=7.9, 4.7 Hz, 1H), 4.78 (ddd, J=12.2, 8.3, 5.3 Hz, 1H), 3.88 (s, 3H), 2.80 (ddd, I=17.3, 13.1, 5.5 Hz, 1H), 2.59-2.51 (m, 1H), 2.12 (qd, J=12.8, 4.5 Hz, 1H), 2.00 (dtd, J=12.8, 5.5, 3.0 Hz, 1H).

LCMS (m/z [M+H]⁺): 286.9

### Example 89: Synthesis of 5-chloro-N-(2,6-dioxopiperidin-3-yl)-1H-pyrrolo[2,3-b]pyridine-3-carboxamide (99)

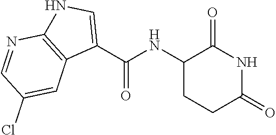 [SMILES: O=C1CCC(NC(=O)c2c[nH]c3ncc(Cl)cc23)C(=O)N1]

This compound was synthesized using the general procedure shown in Reaction Scheme 1 and Synthetic Conditions C, above, (31% yield) using 5-chloro-1H-pyrrolo[2,3-b]pyridine-3-carboxylic acid (20 mg) as a starting material.

¹H NMR (500 MHz, DMSO) δ 12.40 (s, 1H), 10.85 (s, 1H), 8.44 (d, J=2.5 Hz, 1H), 8.39 (d, J=8.4 Hz, 1H), 8.30 (d, J=2.4 Hz, 1H), 8.27 (d, I=2.9 Hz, 1H), 4.79 (ddd, J=12.2, 8.3, 5.3 Hz, 1H), 2.81 (ddd, J=17.3, 13.2, 5.5 Hz, 1H), 2.59-2.52 (m, 1H), 2.11 (qd, J=12.8, 4.4 Hz, 1H), 2.01 (dtd, J=13.0, 5.4, 2.9 Hz, 1H).

LCMS (m/z [M+H]⁺): 307.2

### Example 90. Synthesis of 4-chloro-N-(2,6-dioxopiperidin-3-yl)-7H-pyrrolo[2,3-d]pyrimidine-5-carboxamide (100)

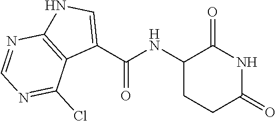 [SMILES: O=C1CCC(NC(=O)c2c[nH]c3ncnc(Cl)c23)C(=O)N1]

This compound was synthesized using the general procedure shown in Reaction Scheme 1 and Synthetic Conditions C, above (8% yield), and 4-chloro-7H-pyrrolo[2,3-d]pyrimidine-5-carboxylic acid (20.0 mg) as a starting material.

¹H NMR (500 MHz, DMSO) δ 10.82 (s, 1H), 8.55 (s, 1H), 8.49 (s, 1H), 8.36 (s, 1H), 8.06 (s, 1H), 4.76 (q, J=8.5 Hz, 1H), 2.78 (ddd, J=18.0, 10.3, 8.4 Hz, 1H), 2.56 (dt, 1=17.2, 3.8 Hz, 1H), 2.06 (h, J=5.0, 4.4 Hz, 2H).

LCMS (m/z [M+H]⁺): 308.0

### Example 91: Synthesis of N-(2,6-dioxopiperidin-3-yl)-1H-pyrazolo[4,3-b]pyridine-3-carboxamide (101)

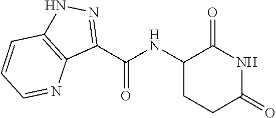 [SMILES: O=C1CCC(NC(=O)c2n[nH]c3cccnc23)C(=O)N1]

This compound was synthesized using the general procedure shown in Reaction Scheme 1 and Synthetic Conditions C, above, (40% yield) using 1H-pyrazolo[4,3-b]pyridine-3-carboxylic acid (20 mg) as a starting material.

¹H NMR (500 MHz, DMSO): δ 14.01 (s, 1H), 10.93 (s, 1H), 9.07 (d, J=7.5 Hz, 1H), 8.69 (dd, J=4.4, 1.3 Hz, 1H), 8.19 (dd, J=8.5, 1.0 Hz, 1H), 7.51 (dd, J=8.5, 4.4 Hz, 1H), 4.92 (ddd, J=7.5, 6.7, 4.2 Hz, 1H), 2.90-2.78 (m, 1H), 2.60-2.54 (m, 1H), 2.25-2.11 (m, 2H).

LCMS (m/z [M+H]⁺): 273.8

### Example 92: Synthesis of N-(2,6-dioxopiperidin-3-yl)-2-acetamido-4,5,6,7-tetrahydro-1-benzothiophene-3-carboxamide (105)

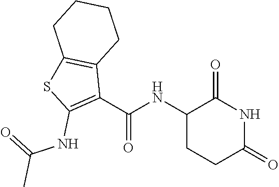 [SMILES: CC(=O)Nc1sc2c(c1C(=O)NC1CCC(=O)NC1=O)CCCC2]

This compound was synthesized using the general procedure shown in Reaction Scheme 1 and Synthetic Conditions B, above (19% yield), with 2-acetamido-4,5,6,7-tetrahydro-1-benzothiophene-3-carboxylic acid as a starting material.

¹H NMR (500 MHz, DMSO) δ 10.99 (s, 1H), 10.82 (s, 1H), 7.99 (d, J=8.4 Hz, 1H), 4.91-4.76 (m, 1H), 2.82 (ddd, J=17.5, 13.1, 6.1 Hz, 1H), 2.73-2.54 (m, 5H), 2.16 (s, 3H), 2.15-2.00 (m, 2H), 1.84-1.67 (m, 4H).

LCMS (m/z [M−H]⁻): 347.8

### Example 93: Synthesis of 5-chloro-N-(2,6-dioxopiperidin-3-yl)thieno[3,4-b]pyridine-7-carboxamide (106)

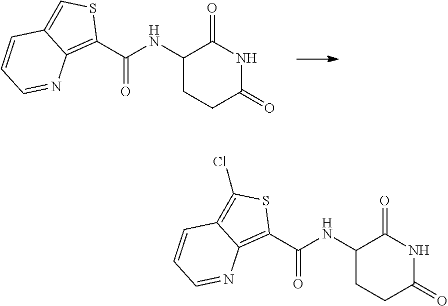 [SMILES: O=C1CCC(NC(=O)c2scc3cccnc23)C(=O)N1 ; O=C1CCC(NC(=O)c2sc(Cl)c3cccnc23)C(=O)N1]

N-chlorosuccinimide (0.059 g, 0.442 mmol, 1.1 eq) was added to a suspension of N-(2,6-dioxopiperidin-3-yl)thieno[3,4-b]pyridine-7-carboxamide (0.116 g, 0.401 mmol) in DMF (5 mL) at RT. The reaction mixture was heated to 60′C and stirred for 3 h. The obtained crude compound was purified by HPLC to give 5-chloro-N-(2,6-dioxopiperidin-3-yl)thieno[3,4-b]pyridine-7-carboxamide (43% yield).

¹H NMR (400 MHz, DMSO) δ 10.97 (s, 1H), 9.51 (d, J=7.4 Hz, 1H), 8.91-8.83 (m, 1H), 8.19 (d, J=8.8 Hz, 1H), 7.41-7.33 (m, 1H), 4.95-4.84 (m, 1H), 2.89-2.74 (m, 1H), 2.62-2.55 (m, 1H), 2.28-2.11 (m, 2H)

LCMS (m/z [M+H]⁺): 323.8

### Example 94: Synthesis of N-(2,6-dioxopiperidin-3-yl)thieno[3,4-b]pyridine-7-carboxamide (107)

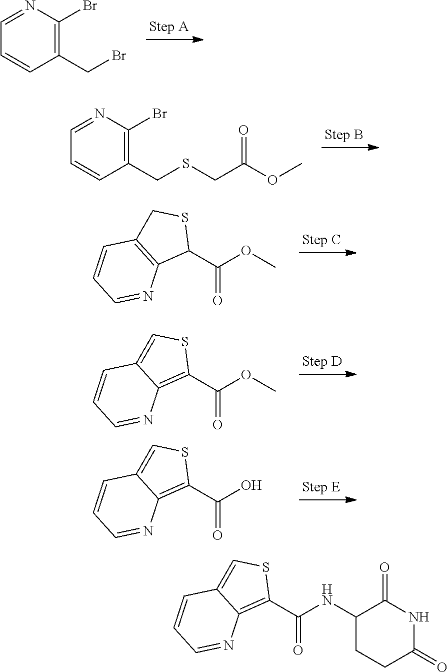

Step A: To an ice-cold solution of 2-bromo-3-(bromomethyl)pyridine 2 (10.5 g, 42.0 mmol) in THF (100 mL) was added methyl thioglycolate (4.089 g, 18.124 mmol) followed by Et₃N under stirring. The mixture was warmed to RT and stirred for further 30 min. The reaction mixture was diluted with water and extracted with DCM, dried over Na₂SO₄, concentrated under reduced pressure and purified by flash column chromatography to give methyl 2-{[(2-bromopyridin-3-yl)methyl]sulfanyl}acetate (53% yield).

Step B: A solution of methyl 2-{[(2-bromopyridin-3-yl)methyl]sulfanyl}acetate (4.5 g, 16.295 mmol) in THF (25 mL) was added slowly to a suspension of KH (1.307 g, 32.591 mmol) and stirred for 20 min at room temperature. The reaction mixture was then cooled to −78° C. and treated with saturated aqueous NH₄Cl solution, warmed to RT, extracted with DCM, dried over Na₂SO₄, concentrated under reduced pressure and purified by flash column chromatography to give methyl 5H,7H-thieno[3,4-b]pyridine-7-carboxylate (56% yield).

Step C: To the stirred solution of methyl 5H,7H-thieno[3,4-b]pyridine-7-carboxylate (3 g, 15.385 mmol) in CHCl₃ (25 mL) was added activated MnO₂ (13.375 g, 153.846 mmol) and the reaction mixture was stirred at RT for 16 h, filtered through celite bed, concentrated under reduced pressure and purified by flash column chromatography to give methyl thieno[3,4-b]pyridine-7-carboxylate (46% yield).

Step D: To a stirred solution of methyl thieno[3,4-b]pyridine-7-carboxylate (1.5 g, 7.772 mmol) in THF:MeOH:H₂O, 4:2:1 (14 mL) was added LiOH.H₂O (1.304 g, 31.088 mmol) at 0° C. and then ice-bath was removed and the mixture was stirred at RT for 2.5 h. Saturated aqueous citric acid solution was added and the product was extracted with 10% MeOH in DCM, dried over Na₂SO₄, concentrated under reduced pressure and purified by HPLC to give thieno[3,4-b]pyridine-7-carboxylic acid (72 mg, 5%).

Step E: Synthesis of N-(2,6-dioxopiperidin-3-yl)thieno[3,4-b]pyridine-7-carboxamide was synthesized using the general procedure shown in Reaction Scheme 1 and Synthetic Conditions C, above (69% yield), and thieno[3,4-b]pyridine-7-carboxylic acid (25.0 mg) as a starting material.

¹H NMR (500 MHz, DMSO) δ 10.95 (s, 1H), 9.71 (d, J=7.4 Hz, 1H), 8.83 (dd, J=4.0, 1.6 Hz, 1H), 8.59 (s, 1H), 8.32 (dd, J=8.8, 1.6 Hz, 1H), 7.29 (dd, J=8.7, 4.0 Hz, 1H), 4.92 (ddd, J=12.7, 7.4, 5.4 Hz, 1H), 2.89-2.79 (m, 1H), 2.60-2.52 (m, 1H), 2.30-2.11 (m, 2H).

LCMS (m/z [M+H]⁺): 290.0

### Example 95: N-(2,6-dioxopiperidin-3-yl)-3-methoxythiophene-2-carboxamide (108)

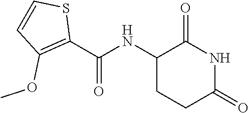 [SMILES: COc1ccsc1C(=O)NC1CCC(=O)NC1=O]

This compound was synthesized using the general procedure shown in Reaction Scheme 1 and Synthetic Conditions C, above, (50% yield) using 3-methoxythiophene-2-carboxylic acid (20 mg) as a starting material.

¹H NMR (500 MHz, DMSO) δ 10.87 (s, 1H), 7.89 (d, J=7.4 Hz, 1H), 7.75 (d, J=5.5 Hz, 1H), 7.14 (d, J=5.5 Hz, 1H), 4.78-4.63 (m, 1H), 3.98 (s, 3H), 3.31 (s, 3H), 2.77 (ddd, J=17.4, 13.1, 6.3 Hz, 1H), 2.54-2.51 (m, 1H), 2.17-2.04 (m, 2H).

LCMS (m/z [M+H]⁺): 268.9

### Example 96: Synthesis of (S)—N-(2,6-dioxopiperidin-3-yl)-4-methoxythiophene-3-carboxamide (109)

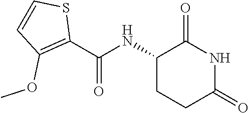 [SMILES: COc1ccsc1C(=O)N[C@H]1CCC(=O)NC1=O]

This compound was synthesized using the general procedure shown in Reaction Scheme 1 and Synthetic Conditions D, above, (42% yield) using 4-methoxythiophene-3-carboxylic acid (20 mg) and (S)-3-aminopiperidine-2,6-dione as a starting material.

¹H NMR (500 MHz, DMSO) δ 10.89 (s, 1H), 8.12 (d, J=7.4 Hz, 1H), 8.09 (d, J=3.6 Hz, 1H), 6.81 (d, J=3.6 Hz, 1H), 4.78-4.65 (m, 1H), 3.89 (s, 3H), 2.84-2.72 (m, 1H), 2.56-2.52 (m, 1H), 2.15-2.06 (m, 2H).

LCMS (m/z [M+H]⁺): 268.9

### Example 97: Synthesis of (R)—N-(2,6-dioxopiperidin-3-yl)-4-methoxythiophene-3-carboxamide (110)

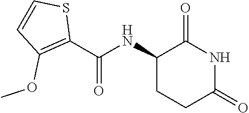 [SMILES: COc1ccsc1C(=O)N[C@@H]1CCC(=O)NC1=O]

This compound was synthesized using the general procedure shown in Reaction Scheme 1 and Synthetic Conditions D, above, (40% yield) using 4-methoxythiophene-3-carboxylic acid (20 mg) and (R)-3-aminopiperidine-2,6-dione as a starting material.

¹H NMR (500 MHz, DMSO) δ 10.89 (s, 1H), 8.12 (d, J=7.4 Hz, 1H), 8.09 (d, J=3.6 Hz, 1H), 6.81 (d, J=3.6 Hz, 1H), 4.76-4.68 (m, 1H), 3.89 (s, 3H), 2.84-2.72 (m, 1H), 2.55-2.52 (m, 1H), 2.14-2.06 (m, 2H).

LCMS (m/z [M+H]⁺): 268.8

### Example 98: Synthesis of N-(2,6-dioxopiperidin-3-yl)-2-(methoxymethyl)thiazole-5-carboxamide (111)

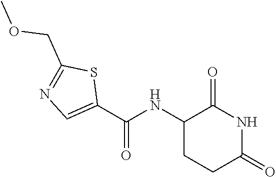 [SMILES: COCc1ncc(C(=O)NC2CCC(=O)NC2=O)s1]

This compound was synthesized using the general procedure shown in Reaction Scheme 1 and Synthetic Conditions C, above (79% yield) using 2-(methoxymethyl)thiazole-5-carboxylic acid (20 mg) as a starting material.

¹H NMR (500 MHz, DMSO) δ 10.89 (s, 1H), 8.95 (d, J=8.3 Hz, 1H), 8.34 (s, 1H), 4.77-4.70 (m, 3H), 4.72 (s, 2H), 3.40 (d, J=17.0 Hz, 3H), 2.79 (ddd, J=17.5, 13.3, 5.6 Hz, 1H), 2.52 (dd, J=5.7, 3.8 Hz, 1H), 2.10 (qd, J=13.0, 4.5 Hz, 1H), 1.99 (dddd, J=11.0, 8.3, 5.5, 2.8 Hz, 1H).

LCMS (m/z [M+H]⁺): 283.9

### Example 99: Synthesis of 2-amino-N-(2,6-dioxopiperidin-3-yl)thiazole-5-carboxamide (112)

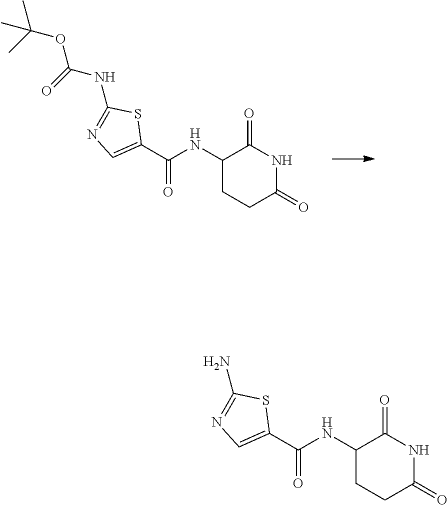 [SMILES: CC(C)(C)OC(=O)Nc1ncc(C(=O)NC2CCC(=O)NC2=O)s1 ; Nc1ncc(C(=O)NC2CCC(=O)NC2=O)s1]

To the suspension of tert-butyl (5-((2,6-dioxopiperidin-3-yl)carbamoyl)thiazol-2-yl)carbamate (71, 30 mg, 0.085 mmol, 1 eq) in DCM (1.5 mL) was added TFA (0.2 mL) and mixture was stirred for 18 h at RT, concentrated under reduced pressure and purified by HPLC to give 2-amino-N-(2,6-dioxopiperidin-3-yl)thiazole-5-carboxamide (yield 37%).

¹H NMR (500 MHz, DMSO) δ 10.81 (s, 1H), 8.35 (d, J=8.4 Hz, 1H), 7.64 (s, 1H), 7.49 (s, 2H), 4.64 (ddd, J=12.5, 8.4, 5.4 Hz, 1H), 2.76 (ddd, J=17.4, 13.3, 5.6 Hz, 1H), 2.53-2.51 (m, 2H), 2.05 (qd, J=12.8, 4.3 Hz, 1H), 1.93 (dddd, J=10.7, 8.1, 5.3, 2.9 Hz, 1H).

LCMS (m/z [M+H]⁺): 255.2

### Example 100: Fluorescence Polarization (FP) Assays

CRBN-DDB1 protein complex was mixed with Cy5-labelled thalidomide and a compound to be tested (the “test compound”). The test solution contained 50 mM Tris pH=7.0, 200 mM NaCl, 0.02% v/v Tween-20, 2 mM DTT, 5 nM Cy5-labelled thalidomide (the tracer), 25 nM CRBN-DDB1 protein, 2% v/v DMSO. The test solution was added to a 384-well assay plate.

The plate was spun-down (1 min, 1000 rpm, 22° C.) and then shaken using a VibroTurbulator for 10 min at room temperature (20-25° C.), with the frequency set to level 3. The assay plate with protein and the tracer was incubated for 60 min at room temperature (20-25° C.) prior to read-out with a plate reader. Read-out (fluorescence polarization) was performed by a Pherastar plate reader, using a Cy5 FP Filterset (590 nm/675 nm).

The FP experiment was carried out with various concentrations of the test compounds in order to measure Kᵢ values.

The Kᵢ values of competitive inhibitors were calculated using the equation based on the IC₅₀ values of relationship between compound concentration and measured fluorescence polarization, the K_{d} value of the Cy5-T and CRBN/DDB1 complex, and the concentrations of the protein and the tracer in the displacement assay (as described by Z. Nikolovska-Coleska et al., Analytical Biochemistry 332 (2004) 261-273).

Fluorescence Polarization (FP) Assay—Results

Compounds are categorized based on their affinity to CRBN defined as Ki. As reported in Table 2 below, the compounds of the present invention interact with CRBN-DDB1 protein within similar affinity range as reported for reference compounds.

TABLE 2
FP assay results for compounds of the present invention, and control
compounds CC-122, lenalidomide and pomalidomide

| | | CRBN binding |
|---|---|---|
| Compound ID | Structure | Ki [μM] |
| CC-122 | 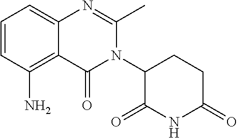 [SMILES: Cc1nc2cccc(N)c2c(=O)n1C1CCC(=O)NC1=O] | A |
| Pomalidomide | 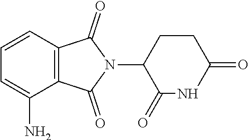 [SMILES: Nc1cccc2c1C(=O)N(C1CCC(=O)NC1=O)C2=O] | A |
| Lenalidomide | 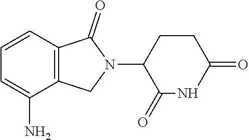 [SMILES: Nc1cccc2c1CN(C1CCC(=O)NC1=O)C2=O] | A |
| 1 | 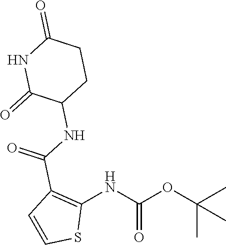 [SMILES: CC(C)(C)OC(=O)Nc1sccc1C(=O)NC1CCC(=O)NC1=O] | C |
| 3 | 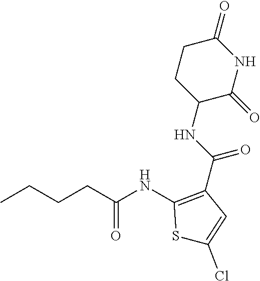 [SMILES: CCCCC(=O)Nc1sc(Cl)cc1C(=O)NC1CCC(=O)NC1=O] | A |
| 4 | 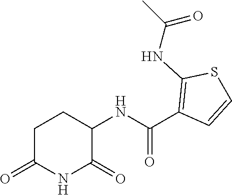 [SMILES: CC(=O)Nc1sccc1C(=O)NC1CCC(=O)NC1=O] | A |
| 5 | 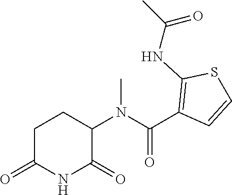 [SMILES: CC(=O)Nc1sccc1C(=O)N(C)C1CCC(=O)NC1=O] | C |
| 6 | 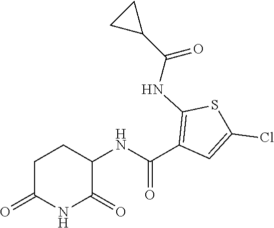 [SMILES: O=C1CCC(NC(=O)c2cc(Cl)sc2NC(=O)C2CC2)C(=O)N1] | A |
| 7 | 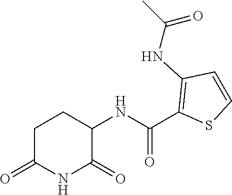 [SMILES: CC(=O)Nc1ccsc1C(=O)NC1CCC(=O)NC1=O] | B |
| 8 | 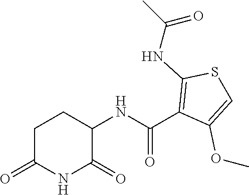 [SMILES: COc1csc(NC(C)=O)c1C(=O)NC1CCC(=O)NC1=O] | B |
| 9 | 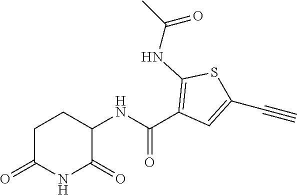 [SMILES: C#Cc1cc(C(=O)NC2CCC(=O)NC2=O)c(NC(C)=O)s1] | A |
| 12 | 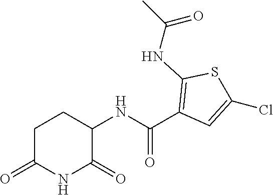 [SMILES: CC(=O)Nc1sc(Cl)cc1C(=O)NC1CCC(=O)NC1=O] | B |
| 13 | 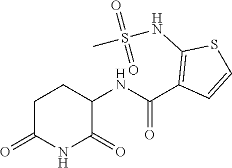 [SMILES: CS(=O)(=O)Nc1sccc1C(=O)NC1CCC(=O)NC1=O] | B |
| 14 | 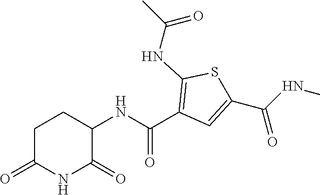 [SMILES: CNC(=O)c1cc(C(=O)NC2CCC(=O)NC2=O)c(NC(C)=O)s1] | B |
| 15 | 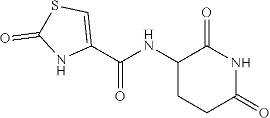 [SMILES: O=C1CCC(NC(=O)c2csc(=O)[nH]2)C(=O)N1] | B |
| 16 | 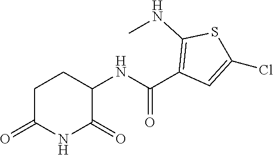 [SMILES: CNc1sc(Cl)cc1C(=O)NC1CCC(=O)NC1=O] | A |
| 17 | 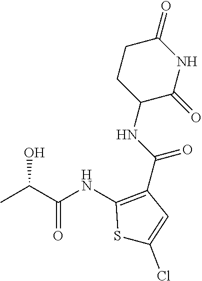 [SMILES: C[C@H](O)C(=O)Nc1sc(Cl)cc1C(=O)NC1CCC(=O)NC1=O] | C |
| 18 | 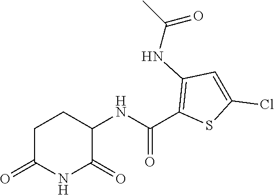 [SMILES: CC(=O)Nc1cc(Cl)sc1C(=O)NC1CCC(=O)NC1=O] | C |
| 19 | 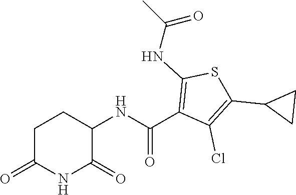 [SMILES: CC(=O)Nc1sc(C2CC2)c(Cl)c1C(=O)NC1CCC(=O)NC1=O] | B |
| 20 | 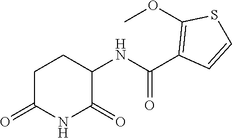 [SMILES: COc1sccc1C(=O)NC1CCC(=O)NC1=O] | A |
| 21 | 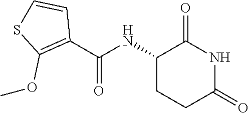 [SMILES: COc1sccc1C(=O)N[C@H]1CCC(=O)NC1=O] | A |
| 22 | 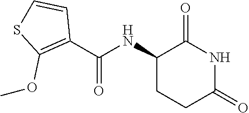 [SMILES: COc1sccc1C(=O)N[C@@H]1CCC(=O)NC1=O] | C |
| 28 | 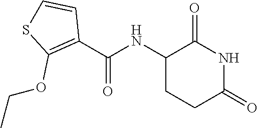 [SMILES: CCOc1sccc1C(=O)NC1CCC(=O)NC1=O] | A |
| 29 | 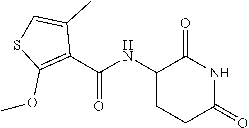 [SMILES: COc1scc(C)c1C(=O)NC1CCC(=O)NC1=O] | B |
| 30 | 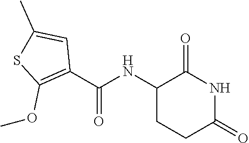 [SMILES: COc1sc(C)cc1C(=O)NC1CCC(=O)NC1=O] | A |
| 32 | 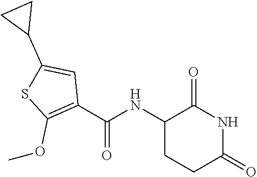 [SMILES: COc1sc(C2CC2)cc1C(=O)NC1CCC(=O)NC1=O] | A |
| 33 | 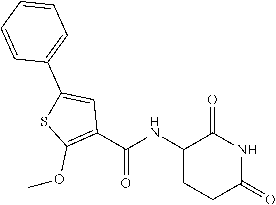 [SMILES: COc1sc(-c2ccccc2)cc1C(=O)NC1CCC(=O)NC1=O] | A |
| 34 | 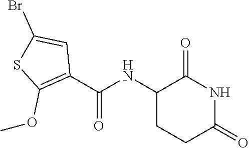 [SMILES: COc1sc(Br)cc1C(=O)NC1CCC(=O)NC1=O] | A |
| 35 | 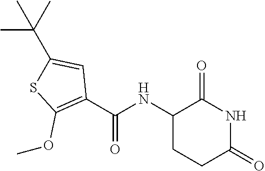 [SMILES: COc1sc(C(C)(C)C)cc1C(=O)NC1CCC(=O)NC1=O] | A |
| 36 | 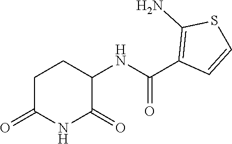 [SMILES: Nc1sccc1C(=O)NC1CCC(=O)NC1=O] | A |
| 37 | 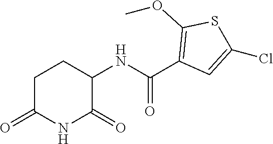 [SMILES: COc1sc(Cl)cc1C(=O)NC1CCC(=O)NC1=O] | A |
| 39 | 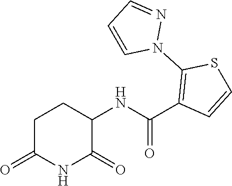 [SMILES: O=C1CCC(NC(=O)c2ccsc2-n2cccn2)C(=O)N1] | C |
| 40 | 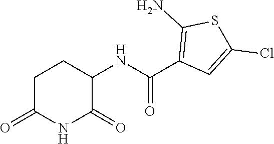 [SMILES: Nc1sc(Cl)cc1C(=O)NC1CCC(=O)NC1=O] | A |
| 42 | 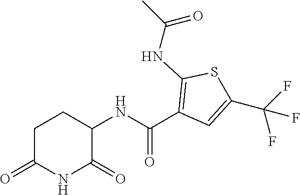 [SMILES: CC(=O)Nc1sc(C(F)(F)F)cc1C(=O)NC1CCC(=O)NC1=O] | B |
| 43 | 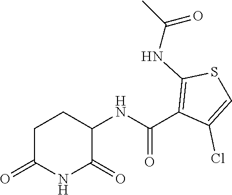 [SMILES: CC(=O)Nc1scc(Cl)c1C(=O)NC1CCC(=O)NC1=O] | C |
| 44 | 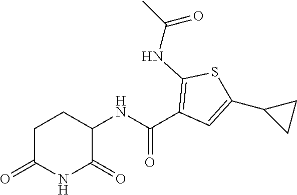 [SMILES: CC(=O)Nc1sc(C2CC2)cc1C(=O)NC1CCC(=O)NC1=O] | A |
| 46 | 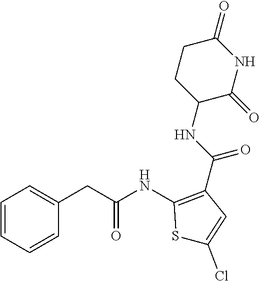 [SMILES: O=C1CCC(NC(=O)c2cc(Cl)sc2NC(=O)Cc2ccccc2)C(=O)N1] | B |
| 47 | 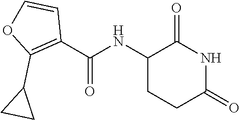 [SMILES: O=C1CCC(NC(=O)c2ccoc2C2CC2)C(=O)N1] | C |
| 49 | 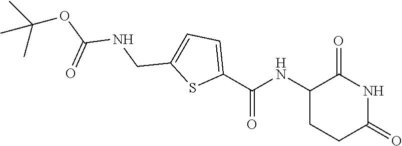 [SMILES: CC(C)(C)OC(=O)NCc1ccc(C(=O)NC2CCC(=O)NC2=O)s1] | A |
| 50 | 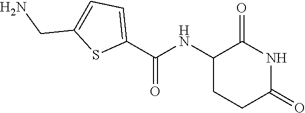 [SMILES: NCc1ccc(C(=O)NC2CCC(=O)NC2=O)s1] | C |
| 51 | 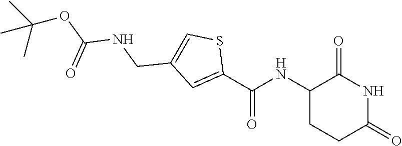 [SMILES: CC(C)(C)OC(=O)NCc1csc(C(=O)NC2CCC(=O)NC2=O)c1] | A |
| 52 | 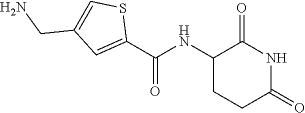 [SMILES: NCc1csc(C(=O)NC2CCC(=O)NC2=O)c1] | C |
| 53 | 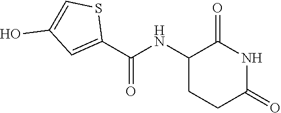 [SMILES: O=C1CCC(NC(=O)c2cc(O)cs2)C(=O)N1] | A |
| 54 | 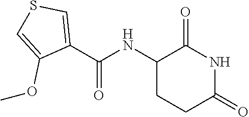 [SMILES: COc1cscc1C(=O)NC1CCC(=O)NC1=O] | A |
| 57 | 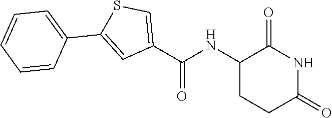 [SMILES: O=C1CCC(NC(=O)c2csc(-c3ccccc3)c2)C(=O)N1] | A |
| 63 | 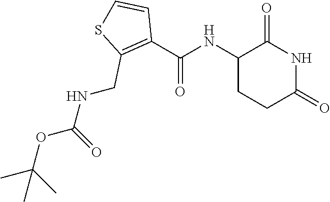 [SMILES: CC(C)(C)OC(=O)NCc1sccc1C(=O)NC1CCC(=O)NC1=O] | C |
| 66 | 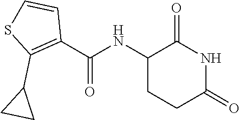 [SMILES: O=C1CCC(NC(=O)c2ccsc2C2CC2)C(=O)N1] | C |
| 68 | 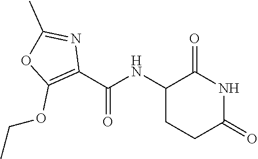 [SMILES: CCOc1oc(C)nc1C(=O)NC1CCC(=O)NC1=O] | C |
| 71 | 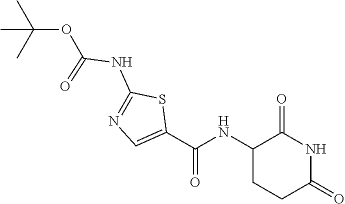 [SMILES: CC(C)(C)OC(=O)Nc1ncc(C(=O)NC2CCC(=O)NC2=O)s1] | A |
| 73 | 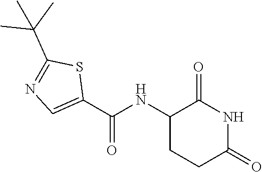 [SMILES: CC(C)(C)c1ncc(C(=O)NC2CCC(=O)NC2=O)s1] | C |
| 74 | 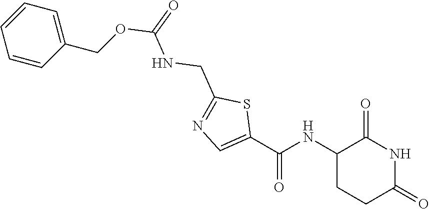 [SMILES: O=C1CCC(NC(=O)c2cnc(CNC(=O)OCc3ccccc3)s2)C(=O)N1] | C |
| 75 | 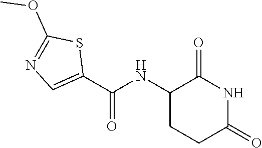 [SMILES: COc1ncc(C(=O)NC2CCC(=O)NC2=O)s1] | C |
| 76 | 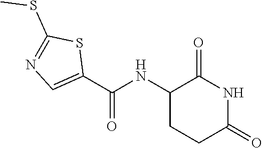 [SMILES: CSc1ncc(C(=O)NC2CCC(=O)NC2=O)s1] | C |
| 77 | 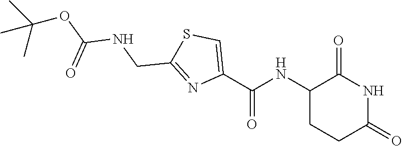 [SMILES: CC(C)(C)OC(=O)NCc1nc(C(=O)NC2CCC(=O)NC2=O)cs1] | B |
| 78 | 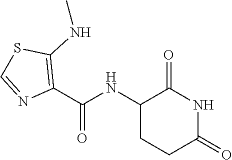 [SMILES: CNc1scnc1C(=O)NC1CCC(=O)NC1=O] | C |
| 79 | 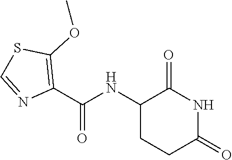 [SMILES: COc1scnc1C(=O)NC1CCC(=O)NC1=O] | B |
| 80 | 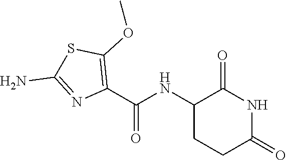 [SMILES: COc1sc(N)nc1C(=O)NC1CCC(=O)NC1=O] | A |
| 82 | 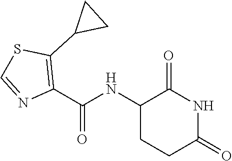 [SMILES: O=C1CCC(NC(=O)c2ncsc2C2CC2)C(=O)N1] | C |
| 96 | 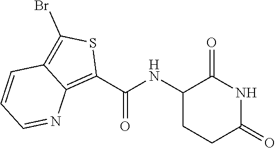 [SMILES: O=C1CCC(NC(=O)c2sc(Br)c3cccnc23)C(=O)N1] | A |
| 97 | 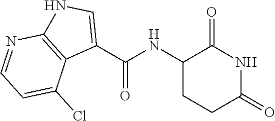 [SMILES: O=C1CCC(NC(=O)c2c[nH]c3nccc(Cl)c23)C(=O)N1] | B |
| 98 | 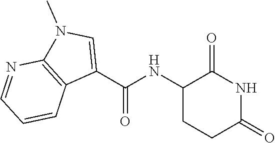 [SMILES: Cn1cc(C(=O)NC2CCC(=O)NC2=O)c2cccnc21] | C |
| 99 | 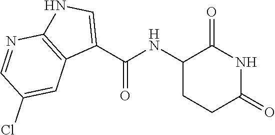 [SMILES: O=C1CCC(NC(=O)c2c[nH]c3ncc(Cl)cc23)C(=O)N1] | B |
| 106 | 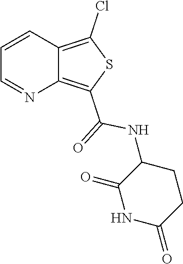 [SMILES: O=C1CCC(NC(=O)c2sc(Cl)c3cccnc23)C(=O)N1] | A |
| 107 | 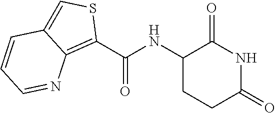 [SMILES: O=C1CCC(NC(=O)c2scc3cccnc23)C(=O)N1] | A |
| 108 | 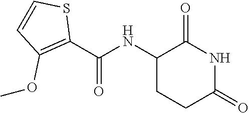 [SMILES: COc1ccsc1C(=O)NC1CCC(=O)NC1=O] | A |
| 109 | 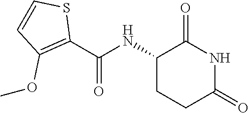 [SMILES: COc1ccsc1C(=O)N[C@H]1CCC(=O)NC1=O] | A |
| 110 | 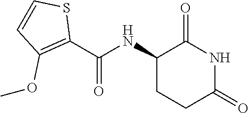 [SMILES: COc1ccsc1C(=O)N[C@@H]1CCC(=O)NC1=O] | B |
| 111 | 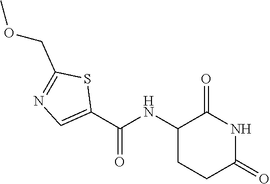 [SMILES: COCc1ncc(C(=O)NC2CCC(=O)NC2=O)s1] | C |
| 112 | 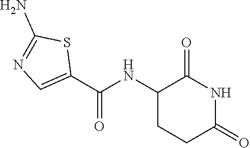 [SMILES: Nc1ncc(C(=O)NC2CCC(=O)NC2=O)s1] | B |

CRBN binding Ki [μm] is indicated as follows:
A < 5 μm
5 μm ≤ B ≤ 10 μm
10 μm < C ≤ 50 μm

### Example 101: CK1α Degradation Assay—Kelly Cell Line

The effect of various compounds of the invention and various reference compounds on CK1α degradation in the Kelly cell line was investigated, using the degradation assay protocol below.

Kelly cells were maintained in RPMI-1640 medium, supplemented with penicillin/streptomycin and 10% Fetal Bovine Serum (FBS). Cells were seeded on 6-well plates, and the compounds to be tested were added at the desired concentration range. Final DMSO concentration was 0.25%. After 24 h incubation (37° C., 5% CO2), cells were washed and cell lysates were prepared using RIPA lysis buffer. The amount of protein was determined via BCA assay, and the appropriate quantity was then loaded on the precast gel for the protein separation. After primary and secondary Ab staining, the membranes were washed and signals developed. The densitometry analysis was implemented to obtain the numeric values used later in the protein level evaluation process.

The compounds tested in this assay were: 54, 109, POMALIDOMIDE, CC-122, and LENALIDOMIDE at the concentrations 1, 10 and 20 μM. In addition, compounds 22, 21, 108 and 110 were tested at 20 μM. The treatment with all compounds was carried out for 24 h.

Densitometry values are normalized to the loading control ((3-ACTIN) and presented as % of DMSO control, using the following labels:

≤25% for 0-25% of CK1α protein reduction,
>25% for 26-74% of CK1α protein reduction,
≥75% for 75-100% of CK1α protein reduction.

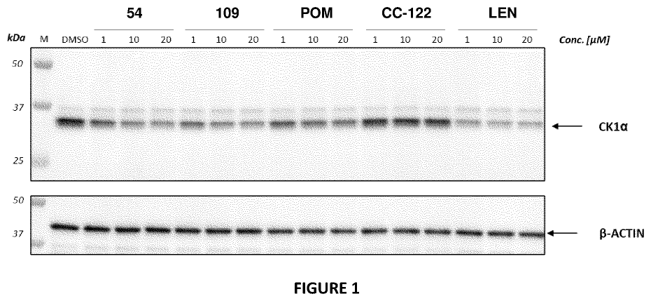

The representative results for compounds: 54, 109, POMALIDOMIDE, CC-122, and LENALIDOMIDE are shown in FIG. 1 and Table 3. The remaining compounds are presented in Table 4. As illustrated by the results, compounds of the present invention induce CK1α degradation in the Kelly cell line. CK1α is also degraded upon treatment with the known compounds LENALIDOMIDE and, to a lesser degree, by POMALIDOMIDE. However, the lack of degradation by CC-122, despite its high affinity to the CRBN protein illustrates, that CK1α-directed activity is not obvious for all chemically-modified thalidomide-based derivatives.

TABLE 3
CK1α degradation in Kelly cell line. Cells were treated with
the compounds: 54, 109, POMALIDOMIDE, CC-122, and
LENALIDOMIDE at the concentrations 1, 10 and 20 μM for 24 h. % of CK1α
protein reduction is provided based on normalized densitometry values.
% of CK1α protein reduction, based on densitometry values

| | DMSO | 54 | | | 109 | | | POMALIDOMIDE | | | CC-122 | | | LENALIDOMIDE | | |
|---|---|---|---|---|---|---|---|---|---|---|---|---|---|---|---|---|
| [μM] | 0.25% | 1 | 10 | 20 | 1 | 10 | 20 | 1 | 10 | 20 | 1 | 10 | 20 | 1 | 10 | 20 |
| CK1α | 0% | >25% | >25% | >25% | >25% | >25% | >25% | >25% | >25% | >25% | ≤25% | ≤25% | ≤25% | >25% | >25% | >25% |

TABLE 4
CK1α degradation in Kelly cell line. Cells were
treated with the compounds: 22, 21, 108, 110 at 20
μM concentration for 24 h. % of CK1α protein
reduction is provided based on normalized densitometry values.

| | CK1α |
|---|---|
| | (24 H, 20 μM) |
| 21 | >25% |
| 22 | >25% |
| 108 | >25% |
| 110 | >25% |

### Example 102: IKZF1 Degradation Assay—H929 Cell Line

The effect of various compounds of the invention and various reference compounds on IKZF1 degradation in the H929 cell line was investigated, using the degradation assay protocol below.

H929 cells were maintained in RPMI-1640 medium, supplemented with penicillin/streptomycin, 10% Fetal Bovine Serum (FBS) and 0.05 mM 2-Mercaptoethanol. Cells were seeded on 6- or 12-well plates, and the compounds to be tested were added at the desired concentration range. Final DMSO concentration was 0.25%. After 6 or 24 h incubation (3TC, 5% CO₂), cells were harvested, washed and cell lysates were prepared using RIPA lysis buffer. The amount of protein was determined via BCA assay, and the appropriate quantity was then loaded on the precast gel for the protein separation. After primary and secondary Ab staining, the membranes were washed and signals developed. The densitometry analysis was implemented to obtain the numeric values used later in the protein level evaluation process.

The compounds tested in this assay were compounds 109, 20, 108, 111, POM, CC-122, LEN, 106, 107 at the concentrations 1, 10 and 20 μM. The remaining compounds, listed in Table 6, were tested at 20 μM. The treatment with all compounds was carried out for 24 h. Densitometry values are normalized to the loading control (β-ACTIN) and presented as % of DMSO control, using the following labels:

≤25% for 0-25% of IKZF1 protein reduction,
>25% for 26-74% of IKZF1 protein reduction,
≥75% for 75-100% of IKZF1 protein reduction.

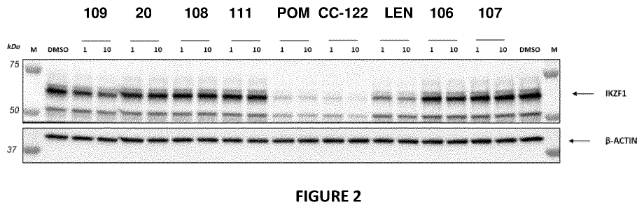

The representative results for compounds 109, 20, 108, 111, POM, CC-122, LEN, 106, 107 are shown in FIG. 2 and Tables 5a and 5b. The remaining compounds are presented in Table 6. As illustrated with these results, the compounds of the present invention present no or low IKZF1 degradation capabilities, in contrast to the LENALIDOMIDE and even more effective POMALIDOMIDE and CC-122.

TABLE 5
IKZF1 degradation in H929 cell line. Cells were treated with the compounds: 109, 20, 108,
111, POM, CC-122, LEN, 106, 107 at the various concentrations (1 and 10 μM) for 24
h. % of IKZF1 α protein reduction is provided based on normalized densitometry values.
% of IKZF1 protein reduction, based on densitometry values

| | DMSO | 109 | | 20 | | 108 | | 111 | | POM | |
|---|---|---|---|---|---|---|---|---|---|---|---|
| [μM] | 0.25% | 1 | 10 | 1 | 10 | 1 | 10 | 1 | 10 | 1 | 10 |
| IKZF1 | 0% | ≤25% | ≤25% | ≤25% | ≤25% | ≤25% | ≤25% | ≤25% | ≤25% | ≥75% | ≥75% |

% of IKZF1 protein reduction, based on densitometry values

| | CC-122 | | LEN | | 106 | | 107 | | DMSO |
|---|---|---|---|---|---|---|---|---|---|
| [μM] | 1 | 10 | 1 | 10 | 1 | 0.25% | 1 | 10 | 0.25% |
| IKZF1 | ≥75% | ≥75% | >25% | >25% | ≤25% | ≤25% | ≤25% | ≤25% | 0% |

TABLE 6
IKZF1 degradation in H929 cell line. Cells were treated with the
compounds at 20 μM concentration for 24 h. % of IKZF1 protein
reduction is provided based on normalized densitometry values.

| | IKZF 1 |
|---|---|
| Compound No | (24 H, 20 μM) |
| 1 | ≤25% |
| 2 | ≤25% |
| 3 | ≤25% |
| 4 | ≤25% |
| 5 | ≤25% |
| 6 | ≤25% |
| 7 | ≤25% |
| 8 | ≤25% |
| 9 | ≤25% |
| 12 | ≤25% |
| 13 | ≤25% |
| 14 | ≤25% |
| 16 | ≤25% |
| 17 | ≤25% |
| 18 | ≤25% |
| 19 | ≤25% |
| 20 | ≤25% |
| 21 | ≤25% |
| 22 | ≤25% |
| 36 | ≤25% |
| 37 | ≤25% |
| 39 | ≤25% |
| 40 | ≤25% |
| 41 | ≤25% |
| 42 | ≤25% |
| 43 | ≤25% |
| 44 | ≤25% |
| 45 | >25% |
| 46 | ≤25% |
| 57 | ≤25% |
| 71 | ≤25% |
| 72 | ≤25% |
| 73 | ≤25% |
| 74 | ≤25% |
| 75 | ≤25% |
| 76 | ≤25% |
| 105 | ≤25% |
| 106 | ≤25% |
| 108 | ≤25% |
| 109 | ≤25% |
| 110 | ≤25% |
| 111 | ≤25% |
| LENALIDOMIDE | >25% |
| POMALIDOMIDE | ≥75% |

### Example 103: IKZF3 Degradation Assay—H929 Cell Line

The effect of various compounds of the invention and various reference compounds on IKZF3 degradation in the H929 cell line was investigated, using the degradation assay protocol below.

H929 cells were maintained in RPMI-1640 medium, supplemented with penicillin/streptomycin, 10% Fetal Bovine Serum (FBS) and 0.05 mM 2-Mercaptoethanol. Cells were seeded on 6- or 12-well plates, and the compounds to be tested were added at the desired concentration range. Final DMSO concentration was 0.25%. After 24 h incubation (3TC, 5% CO₂), cells were harvested, washed and cell lysates were prepared using RIPA lysis buffer. The amount of protein was determined via BCA assay, and the appropriate quantity was then loaded on the precast gel for the protein separation. After primary and secondary Ab staining, the membranes were washed and signals developed. The densitometry analysis was implemented to obtain the numeric values used later in the protein level evaluation process.

The compounds tested in this assay were: compounds 109, 20, 108, 111, POM, CC-122, LEN, 106, 107 at the concentrations 1, 10 and 20 μM. The treatment with all compounds was carried out for 24 h.

Densitometry values are normalized to the loading control ((3-ACTIN) and presented as % of DMSO control, using the following labels:

≤25% for 0-25% of IKZF3 protein reduction,
>25% for 26-74% of IKZF3 protein reduction,
≥75% for 75-100% of IKZF3 protein reduction.

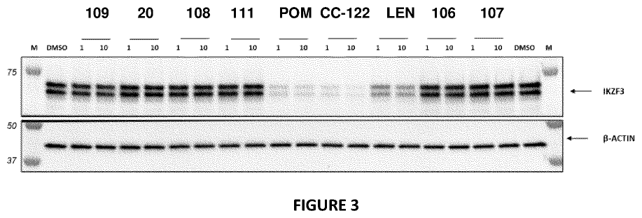

The representative results for compounds 109, 20, 108, 111, POM, CC-122, LEN, 106, 107 are shown in FIG. 3 and Tables 7a and 7b. As illustrated in this Figure, the compounds of the present invention present no IKZF3 degradation efficiency, in contrast to the LENALIDOMIDE and more potent POMALIDOMIDE and CC-122.

TABLE 7
KZF3 degradation in H929 cell line. Cells were treated with the compounds: 109, 20,
108, 111, POM, CC-122, LEN, 106, 107 at the concentrations 1 and 10 μM for 24
h. % of IKZF3 protein reduction is provided based on normalized densitometry values.
% of IKZF3 protein reduction, based on densitometry values

| | DMSO | 109 | | 20 | | 108 | | 111 | | POM | |
|---|---|---|---|---|---|---|---|---|---|---|---|
| [μM] | 0.25% | 1 | 10 | 1 | 10 | 1 | 10 | 1 | 10 | 1 | 10 |
| IKZF3 | 0% | ≤25% | ≤25% | ≤25% | ≤25% | ≤25% | ≤25% | ≤25% | ≤25% | ≥75% | ≥75% |

% of IKZF3 protein reduction, based on densitometry values

| | CC-122 | | LEN | | 106 | | 107 | | DMSO |
|---|---|---|---|---|---|---|---|---|---|
| [μM] | 1 | 10 | 1 | 10 | 1 | 0.25% | 1 | 10 | 0.25% |
| IKZF3 | ≥75% | ≥75% | ≥25% | ≥25% | ≤25% | ≤25% | ≤25% | ≤25% | 0% |

In summary, the compounds of invention are capable of potent degradation of CK1α, a disease relevant protein kinase. By contrast, presented neosubstrates IKZF1, IKZF3 degradation tests results for the compounds of the present invention show no to low degradation of the proteins by the compounds, as opposed to the known CK1α degraders, Lenalidomide and Pomalidomide. This innovative profile renders the compounds of the present invention useful as more selective CK1α degraders.

### ABBREVIATIONS AND DEFINITIONS

A list of the abbreviations used in the present application is shown in Table 8, below:

TABLE 8
Abbreviations

| Abbreviation | Meaning |
|---|---|
| CRBN | Cereblon |
| CRL | Cullin RING Ligase |
| CMA | Cereblon Modulating Agent |
| Cy5-T | Cy5-labelled thalidomide |
| DDB1 | damaged DNA binding protein 1 |
| CUL4 | Cullin-4 |
| RBX1 | RING-Box Protein 1 |
| Bn | benzyl |
| Tris | Tris(hydroxymethyl)aminomethane |
| DMSO | Dimethylsulfoxide |
| THF | tetrahydrofuran |
| DMF | dimethylformamide |
| DMA | dimethylacetamide |
| DMAP | 4-dimethylaminopyridine |
| EDC | 1-ethyl-3-(3-dimethylaminopropyl)carbodiimide |
| HPLC | High performance liquid chromatography |
| NCS | N-chlorosuccinimide |
| DIPEA | N,N-diisopropylethylamine |
| MTBE | Methyl tert-butyl ether |
| HATU | Hexafluorophosphate Azabenzotriazole Tetramethyl |
| | Uronium (1-[Bis(dimethylamino)methylene]-1H- |
| | 1,2,3-triazol[4,5-b]pyridinium-2-oxide |
| | hexafluorophosphate) |
| m-CPBA | meta-chloroperbenzoic acid |
| MTBE | methyl tert butyl ether |
| Pd(dppf)Cl₂ CH₂Cl₂ | [1,1′-bis(diphenylphosphino)ferrocene]dichloro- |
| | palladium(II), complex with dichloromethane |
| DTT | dithiothreitol |
| NK cells | Natural killer cells |
| ADCC | antibody-mediated cellular cytotoxicity |
| GVHD | Graft versus host disease |

As used herein, the term “room temperature” means a temperature of between 20° C. and 25° C.

As used herein, the term “small molecule” means an organic compound with a molecular weight of less than 900 Daltons.

### Embodiments of the Invention

1. A compound of Formula (Ia) or (Ib):

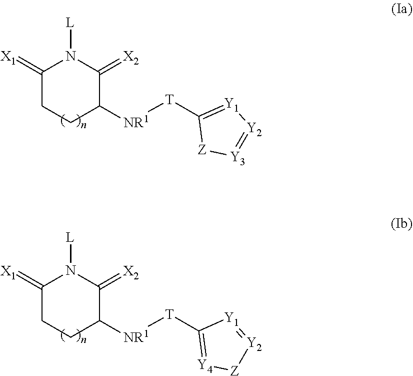

wherein
each of X₁ and X₂ is independently O or S;
Z is S or NR²;
T is C═O or SO₂;
each of Y₁, Y₂, Y₃, and Y₄ is independently N or CR, wherein at least one of Y₁, Y₂ and Y₃ in Formula (Ia) is CR, and at least one of Y₁, Y₂ and Y₄ in Formula (Ib) is CR;
n is 0, 1 or 2;
L is hydrogen, alkyl, alkenyl, aryl, heteroaryl, benzyl, haloalkyl, haloalkenyl, —C(O)R″, —C(O)OR″, —C(O)NH₂, —C(O)NHR″, —C(O)NR″₂, —OR″, —NR″₂, or —S(O)₂R″;
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NR″C(O)R″, —NR″C(O)CH(OH)R″, —NR″C(O)OR″, —NR″SO₂R″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, or —S(O)₂R″, —S(O)₂OR″, —S(O)₂NH₂, —S(O)₂NHR″, or —S(O)₂NR″₂;
each R″ is independently hydrogen, alkyl, cycloalkyl, alkenyl, cycloalkenyl, aryl, heteroaryl, or benzyl;
R² is hydrogen, alkyl, cycloalkyl, alkenyl, cycloalkenyl, alkynyl, aryl, heteroaryl, benzyl, haloalkyl, haloalkenyl, —NH₂, —NHR″, —NR″₂, —NR″C(O)R″, —N[C(O)R″]₂, —NR″C(O)OR″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, or —S(O)₂R″, —S(O)₂OR″, —S(O)₂NH₂, —S(O)₂NHR″, or —S(O)₂NR″₂;
R¹ is hydrogen, alkyl, cycloalkyl, alkenyl, cycloalkenyl, aryl, heteroaryl, or benzyl;
wherein
(i) when each of Y₁, Y₂ and Y₃ in Formula (Ia) is CR, or each of Y₁, Y₂ and Y₄ in Formula (Ib) is CR, then at least one of R is not H;
(ii) when Z is S in Formula (Ib), Y₁ is not C—COOH;
(iii) when Z is NR₂ in Formula (Ib), Y₁, Y₂ and Y₄ are CR; and
(iv) when Z is NR₂ in Formula (Ia), Y₂ and Y₃ are CR.
   2. The compound of embodiment 1, having the structure:

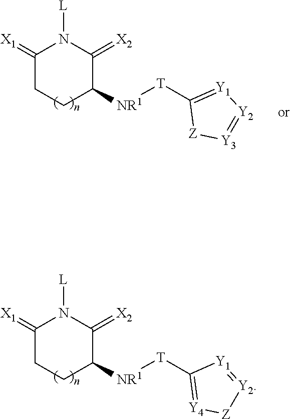

3. The compound of embodiment 1, having the structure:

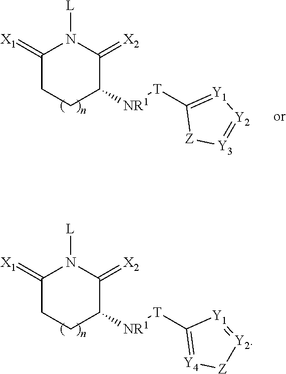

4. The compound of any preceding embodiment, wherein T is C═O.
5. The compound of any one of embodiments 1-3, wherein T is SO₂.
6. The compound of any preceding embodiment, wherein Z is NR².
7. The compound of any preceding embodiment, wherein R² is alkyl, benzyl, or —N[C(O)R″]₂.
8. The compound of any one of embodiments 1-5, wherein Z is S.
9. The compound of any one of embodiments 1-8, wherein L is hydrogen, alkyl, alkenyl, aryl, heteroaryl, benzyl, —OR″, —NR″₂, or —S(O)₂R″; optionally wherein L is hydrogen, alkyl, alkenyl, aryl, heteroaryl, or benzyl.
10. The compound of embodiment 9, wherein L is hydrogen.
11. The compound of any preceding embodiment, wherein the compound is of Formula (Ia), wherein one of Y₁, Y₂ and Y₃ is N, and the remaining two of Y₁, Y₂ and Y₃ are each CR.
12. The compound of embodiment 11, wherein Y₁ is N, and Y₂ and Y₃ are CR.
13. The compound of embodiment 11, wherein Y₂ is N, and Y₁ and Y₃ are CR; and Z is S.
14. The compound of embodiment 11, wherein Y₃ is N; Y₁ and Y₂ are CR; and Z is S.
15. The compound of any one of embodiments 1-10, wherein the compound is of Formula (Ia), wherein one of Y₁, Y₂ and Y₃ is CR, the remaining two of Y₁, Y₂ and Y₃ are each N; and Z is S.
16. The compound of embodiment 15, wherein Y₁ is CR; Y₂ and Y₃ are N.
17. The compound of embodiment 15, wherein Y₂ is CR; Y₁ and Y₃ are N.
18. The compound of embodiment 15, wherein Y₃ is CR, and Y₁ and Y₂ are N.
19. The compound of any one of embodiments 1-10, wherein the compound is of Formula (Ia) and Y₂ and Y₃ are each CR.
20. The compound of embodiment 19, wherein
Y₁ is —C—NHC(O)R″,
Y₂ is CH, and
Y₃ is CH or CCl.
   21. The compound of embodiment 20, wherein:
L is hydrogen;
Z is 5;
R¹ is H;
T is C═O;
Y₁ is —C—NHC(O)R″;
Y₂ is CH; and
Y₃ is CH.
   22. The compound of any one of embodiments 1-10, wherein the compound is of Formula (Ib), wherein one of Y₁, Y₂ and Y₄ is N and the remaining two of Y₁, Y₂ and Y₄ are each CR, and wherein Z is S.
   23. The compound of embodiment 22, wherein Y₁ is N, and Y₂ and Y₄ are CR.
   24. The compound of embodiment 22, wherein Y₂ is N, and Y₁ and Y₄ are CR.
   25. The compound of embodiment 22, wherein Y₄ is N, and Y₁ and Y₂ are CR.
   26. The compound of any one of embodiments 1-10, wherein the compound is of Formula (Ib), wherein one of Y₁, Y₂ and Y₄ is CR and the remaining two of Y₁, Y₂ and Y₄ are each N, and wherein Z is S.
   27. The compound of embodiment 26, wherein Y₁ is CR, and Y₂ and Y₄ are N.
   28. The compound of embodiment 26, wherein Y₂ is CR, and Y₁ and Y₄ are N.
   29. The compound of embodiment 26, wherein Y₄ is CR, and Y₁ and Y₂ are N.
   30. The compound of any one of embodiments 1-10, wherein the compound is of Formula (Ib) and Y₁, Y₂ and Y₄ are each CR.
   31. The compound of embodiment 30, wherein each R is independently hydrogen, halogen, alkyl, cycloalkyl, haloalkyl, heteroaryl, —OR″, —N[C(O)R″]₂, —NR″C(O)R″, —NHC(O)OR″, —NHR″, —NH₂, or —NHSO₂R″CN;
optionally wherein each R″ is independently alkyl, cycloalkyl, aryl or benzyl.
   32. The compound of embodiment 31, wherein:
L is hydrogen;
Z is S;
R¹ is H;
T is C═O;
Y₁ is CH, C—OR″, CCl, C—CN, or C—NHC(O)R″;
Y₂ is CH, CCl, C-alkyl, C-cycloalkyl, or C-haloalkyl; and
Y₄ is CH, C—OR″, C—NHC(O)R″, C—NHC(O)OR″, C—NHR″, C—NH₂, or C—NHSO₂R″;
wherein, when 1¹₁ is CCl, then Y₂ is CH, C-alkyl, C-cycloalkyl, or C-haloalkyl; optionally wherein each R″ is independently alkyl, cycloalkyl, aryl or benzyl.
   33. The compound of embodiment 32, wherein:
Y₁ is CH;
Y₂ is CH or CCl; and
Y₄ is C—OR″ or C—NH₂, optionally C—OMe or C—NH₂.
   34. The compound of any one of embodiments 1-31, wherein each R is independently hydrogen, halogen, alkyl, cycloalkyl, haloalkyl, heteroaryl, —NR″C(O)R″, NR″C(O)OR″, —NR″C(O)CH(OH)R″, —NHR″, —NH₂, —OR″, —CN, —C(O)NR″₂, or —NR″SO₂R″.
   35. The compound of embodiment 34, wherein each R is independently hydrogen, halogen, alkyl, cycloalkyl, haloalkyl, —OR″, —CN, —NHC(O)R″, —NHC(O)OR″, —NHR″, —NH₂ or —NHSO₂R″.
   36. The compound of embodiment 34 or 35, wherein each R″ is independently alkyl, cycloalkyl, aryl or benzyl.
   37. A compound of Formula (IIa) or (IIb):

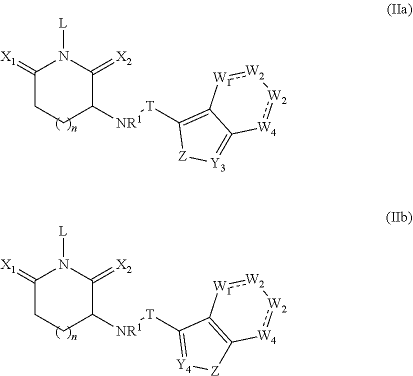

wherein
each of X₁ and X₂ is independently O or S;
Z is O, S or NR²;
T is C═O or SO₂;
Y₃ is N or CR;
Y₄ is N or CR;
[symbol] indicates a single or double bond, wherein
   when each [symbol] is a double bond, each of W₁, W₂, W₃ and W₄ is independently N or CR′, wherein at least one of W₁, W₂, W₃ and W₄ is N, and
   when each [symbol] is a single bond, W₁, W₂, W₃ and W₄ are each CR′2;
n is 0, 1 or 2;
L is hydrogen, alkyl, alkenyl, aryl, heteroaryl, benzyl, haloalkyl, haloalkenyl, —C(O)R″, —C(O)OR″, —C(O)NH₂, —C(O)NHR″, —C(O)NR″₂, —OR″, —NR″₂, or —S(O)₂R″;
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NR″C(O)R″, —NR″C(O)CH(OH)R″, —NR″C(O)OR″, —NR″SO₂R″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, or —S(O)₂R″, —S(O)₂OR″, —S(O)₂NH₂, —S(O)₂NHR″, or —S(O)₂NR″₂;
each R′ is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NR″C(O)R″, —NR″C(O)CH(OH)R″, —NR″C(O)OR″, —NR″SO₂R″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, or —S(O)₂R″, —S(O)₂OR″, —S(O)₂NH₂, —S(O)₂NHR″, or —S(O)₂NR″₂;
each R″ is independently hydrogen, alkyl, cycloalkyl, alkenyl, cycloalkenyl, aryl, heteroaryl, or benzyl;
R² is hydrogen, alkyl, cycloalkyl, alkenyl, cycloalkenyl, alkynyl, aryl, heteroaryl, benzyl, haloalkyl, haloalkenyl, —NH₂, —NHR″, —NR″₂, —NR″C(O)R″, —N[C(O)R″]₂, —NR″C(O)OR″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, or —S(O)₂R″, —S(O)₂OR″, —S(O)₂NH₂, —S(O)₂NHR″, or —S(O)₂NR″₂; and
R¹ is hydrogen, alkyl, cycloalkyl, alkenyl, cycloalkenyl, aryl, heteroaryl, or benzyl.
   38. The compound embodiment 37, having the structure:

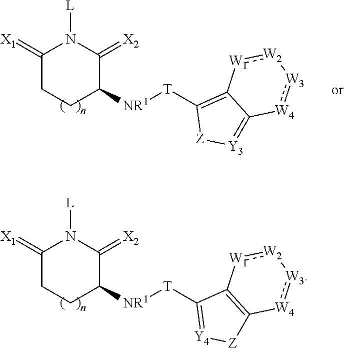

39. The compound embodiment 37, having the structure:

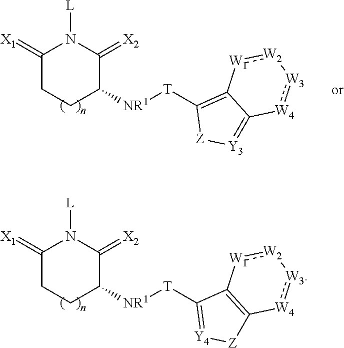

40. The compound of any one of embodiments 37-39, wherein Z is O.
41. The compound of any one of embodiments 37-39, wherein Z is S.
42. The compound of any one of embodiments 37-39, wherein Z is NR².
43. The compound of any one of embodiments 37-42, wherein T is C═O.
44. The compound of any one of embodiments 37-42, wherein T is SO₂.
45. The compound of any one of embodiments 37-44, wherein L is hydrogen, alkyl, alkenyl, aryl, heteroaryl, benzyl, —OR″, —NR″₂, or —S(O)₂R″; optionally wherein L is hydrogen, alkyl, alkenyl, aryl, heteroaryl, or benzyl.
46. The compound of embodiment 45, wherein L is hydrogen.
47. The compound of any one of embodiments 37-46, wherein Y₃ is N.
48. The compound of any one of embodiments 37-46, wherein Y₃ is CR.
49. The compound of any one of embodiments 37-46, wherein Y₄ is N.
50. The compound of any one of embodiments 37-46, wherein Y₄ is CR.
51. The compound of any one of embodiments 37-50, wherein each [symbol] is a double bond or wherein each [symbol] is a single bond.
52. The compound of embodiment 51, wherein each [symbol] is a double bond.
53. The compound of embodiment 52, wherein one of W₁, W₂, W₃ and W₄ is N, and the remaining three of W₁, W₂, W₃ and W₄ are each CR′.
54. The compound of embodiment 52, wherein two of W₁, W₂, W₃ and W₄ is N, and the remaining two of W₁, W₂, W₃ and W₄ are each CR′.
55. The compound of embodiment 52, wherein one of W₁, W₂, W₃ and W₄ is CR′, and the remaining three of W₁, W₂, W₃ and W₄ are each N.
56. The compound of embodiment 51, wherein each [symbol] is a single bond.
57. The compound of any one of embodiments 37-56, wherein each R is independently hydrogen, halogen or —NR″C(O)R″.
58. The compound of any one of embodiments 37-57, wherein each R′ is hydrogen.
59. The compound of any preceding embodiment, wherein X₁ and X₂ are O.
60. The compound of any one of embodiments 1-58 wherein X₁ is O and X₂ is S.
61. The compound of any one of embodiments 1-58, wherein X₁ is S and X₂ is O.
62. The compound of any one of embodiments 1-58, wherein X₁ and X₂ are S.
63. The compound of any preceding embodiment, wherein n is 0.
64. The compound of any one of embodiments 1-62, wherein n is 1.
65. The compound of any one of embodiments 1-62, wherein n is 2.
66. A compound of any one of the preceding embodiments, for use as a cereblon binder.
67. A pharmaceutical composition comprising a compound of any one of embodiments 1-65.
68. A compound of any one of embodiments 1-65, or a composition according to embodiment 67, for use in medicine.
69. A compound of any one of embodiments 1-65, or a composition according to embodiment 67, for use in immune-oncology.
70. A compound of any one of embodiments 1-65, or a composition according to embodiment 67, for use in the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders.
71. A method for the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders;
wherein the method comprises administering to a patient in need thereof an effective amount of compound of any one of embodiments 1-65 or a composition according to embodiment 67.
   72. The method of embodiment 71, further comprising administering at least one additional active agent to the patient.
   73. A combined preparation of a compound of any one of embodiments 1-65 and at least one additional active agent, for simultaneous, separate or sequential use in therapy.
   74. The combined preparation of embodiment 73, or the method of embodiment 72, wherein the at least one additional active agent is an anti-cancer agent or an agent for the treatment of an autoimmune disease.
   75. The combined preparation of any one of embodiments 73-74, or the method of embodiment 72 or 74, wherein the at least one additional active agent is a small molecule, a peptide, an antibody, a corticosteroid, or a combination thereof.
   76. The combined preparation or method of embodiment 75, wherein the at least one additional active agent is at least one of bortezomib, dexamethasone, and rituximab.
   77. The combined preparation of any one of embodiments 73-76 wherein the therapy is the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders.

1. A compound of Formula (Ia) or (Ib): 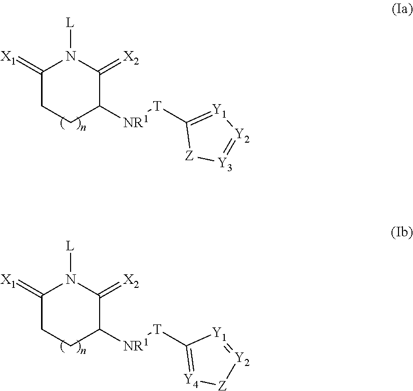
or a pharmaceutically acceptable salt or tautomer thereof,
wherein
each of X₁ and X₂ is independently O or S;
Z is O, S or NR²;
T is C═O or SO₂;
each of Y₁, Y₂, Y₃, and Y₄ is independently N or CR,
wherein at least one of Y₁, Y₂ and Y₃ in Formula (Ia) is CR, and at least one of Y₁, Y₂ and Y₄ in Formula (Ib) is CR;
n is 0, 1 or 2;
L is hydrogen, alkyl, alkenyl, aryl, heteroaryl, benzyl, haloalkyl, haloalkenyl, —C(O)R″, —CH₂C(O)OR″, —C(O)OR″, —C(O)NH₂, —C(O)NHR″, —C(O)NR″₂, —OR″, —NR″₂, or —S(O)₂R″;
each R is independently hydrogen, halogen, alkyl, cycloalkyl, heterocycloalkyl, alkenyl, cycloalkenyl, alkynyl, aryl, heteroaryl, benzyl, haloalkyl, haloalkenyl, —NH₂, —NHR″, —NR″₂, —CH₂NR″₂, —NR″C(O)R″, —NR″C(O)CH₂NR″₂, —NR″C(O)CH₂-heterocycloalkyl, —NR″C(O)CH(OH)R″, —CH₂NR″C(O)OR″, —NR″C(O)OR″, —NR″SO₂R″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —NHC(S)NHR″, SR″, or —S(O)₂R″, —S(O)₂OR″, —S(O)₂NH₂, —S(O)₂NHR″, or —S(O)₂NR″₂;
each R″ is independently hydrogen, alkyl, cycloalkyl, alkenyl, cycloalkenyl, aryl, heteroaryl, or benzyl;
R² is hydrogen, alkyl, cycloalkyl, alkenyl, cycloalkenyl, alkynyl, aryl, heteroaryl, benzyl, haloalkyl, haloalkenyl, —NH₂, —NHR″, —NR″₂, —NR″C(O)R″, —N[C(O)R″]₂, —NR″C(O)OR″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, or —S(O)₂R″, —S(O)₂OR″, —S(O)₂NH₂, —S(O)₂NHR″, or —S(O)₂NR″₂;
R¹ is hydrogen, alkyl, cycloalkyl, alkenyl, cycloalkenyl, aryl, heteroaryl, or benzyl;
wherein when Z is O, then Y₂ is CR′ and
wherein when the compound is of Formula (Ia), then
(i) when each of Y₁, Y₂ and Y₃ is CR, then at least one of R is not H;
(ii) when Z is NR², then Y₂ and Y₃ are CR;
(iii) when Z is S, then Y₁ is not C—OMe and Y₂ is not C—OMe;
(iv) when Z is S and Y₁ is C—NHCOMe, then Y₃ is not C—CH₂NR″C(O)OR″;
(v) when Z is S and Y₁ is N, then Y₂ is not C—H, C-aryl or C—C(O)OR″; and
(vi) when Z is S and Y₂ is N, then Y₃ is C—NH₂, C—NHR″, C—NR″₂, C—NR″C(O)OR″, C—CH₂NR″C(O)OR″, C-haloalkyl, C-^{t}Butyl, C—OR″, C—COOR″ or C—SR″; wherein when Y₃ is C—NH₂, C—NHR″ or C—NR″₂, then Y₁ is C—H;
and when the compound is of Formula (Ib), then:
(vii) when each of Y₁, Y₂ and Y₄ is CR, then at least one of R is not H;
(viii) when Z is S, then Y₁ is not C—COOH or C—NHC(O)Me, and Y₄ is not C—Br;
(ix) when Z is S and Y₂ is C—Br, then Y₄ is C—OR″
(x) when Z is S, Y₁ is N and Y₂ is C—H or C—NH₂, then Y₄ is not C—H
(xi) when Z is S and Y₁ is N, then Y₂ is not C-halogen, C-alkyl, C-cycloalkyl, C-aryl, C-heteroaryl, C—CH₂NH₂, C—COOalkyl, or C—NHC(O)alkyl; (xii) when Z is NR₂, then Y₁, Y₂ and Y₄ are CR.

2. The compound of claim 1, having the structure:

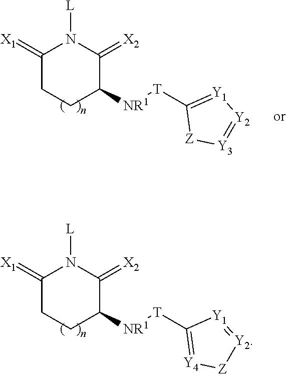

3. The compound of claim 1, having the structure:

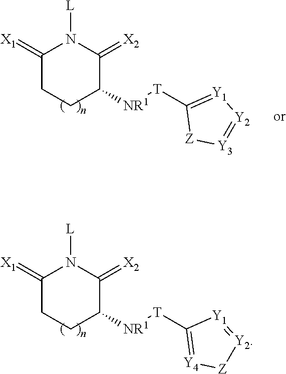

4. The compound of any preceding claim, wherein each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NR″C(O)R″, —NR″C(O)CH(OH)R″, —NR″C(O)OR″, —NR″SO₂R″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, or —S(O)₂R″, —S(O)₂OR″, —S(O)₂NH₂, —S(O)₂NHR″, or —S(O)₂NR″₂.

5. The compound of any preceding claim, wherein T is C═O.

6. The compound of any one of claims 1-4, wherein T is SO₂.

7. The compound of any preceding claim, wherein Z is S or NR².

8. The compound of claim 7, wherein Z is NR².

9. The compound of any preceding claim, wherein R² is alkyl, benzyl, or —N[C(O)R″]₂.

10. The compound of claim 7, wherein Z is S.

11. The compound of any preceding claim, wherein L is hydrogen, alkyl, alkenyl, haloalkyl, haloalkenyl, —C(O)R″, —CH₂C(O)OR″, —C(O)OR″, —C(O)NH₂, —C(O)NHR″, —C(O)NR″₂, —OR″, —NR″₂, or —S(O)₂R″.

12. The compound of any one of claims 1-10, wherein L is hydrogen, alkyl, alkenyl, —CH₂C(O)OR″, —OR″, —NR″₂, or —S(O)₂R″; optionally wherein L is hydrogen, alkyl, or alkenyl.

13. The compound of claim 12, wherein L is hydrogen, alkyl, —CH₂C(O)OR″ or —OR″.

14. The compound of claim 13, wherein L is hydrogen.

15. The compound of any preceding claim, wherein the compound is of Formula (Ia), wherein one of Y₂ and Y₃ is N, and the remaining two of Y₁, Y₂ and Y₃ are each CR.

16. The compound of claim 15, wherein Y₁ is N, and Y₂ and Y₃ are CR.

17. The compound of claim 15, wherein Y₂ is N, and Y₁ and Y₃ are CR; and Z is S.

18. The compound of claim 15, wherein Y₃ is N; Y₁ and Y₂ are CR; and Z is S.

19. The compound of any one of claims 1-14, wherein the compound is of Formula (Ia), wherein one of Y₁, Y₂ and Y₃ is CR, the remaining two of Y₁, Y₂ and Y₃ are each N; and Z is S.

20. The compound of claim 19, wherein Y₁ is CR; Y₂ and Y₃ are N.

21. The compound of claim 19, wherein Y₂ is CR; Y₁ and Y₃ are N.

22. The compound of claim 19, wherein Y₃ is CR, and Y₁ and Y₂ are N.

23. The compound of any one of claims 1-14, wherein the compound is of Formula (Ia) and Y₁, Y₂ and Y₃ are each CR.

24. The compound of claim 23, wherein
Y₁ is —C—NHC(O)R″,
Y₂ is CH, and
Y₃ is CH or CCl.

25. The compound of claim 24, wherein:
L is hydrogen;
Z is S;
R¹ is H;
T is C═O;
Y₁ is —C—NHC(O)R″;
Y₂ is CH; and
Y₃ is CH.

26. The compound of any one of claims 1-14, wherein the compound is of Formula (Ib), wherein one of Y₁, Y₂ and Y₄ is N and the remaining two of Y₁, Y₂ and Y₄ are each CR, and wherein Z is S.

27. The compound of claim 26, wherein Y₁ is N, and Y₂ and Y₄ are CR.

28. The compound of claim 26, wherein Y₂ is N, and Y₁ and Y₄ are CR.

29. The compound of claim 26, wherein Y₄ is N, and Y₁ and Y₂ are CR.

30. The compound of any one of claims 1-14, wherein the compound is of Formula (Ib), wherein one of Y₁, Y₂ and Y₄ is CR and the remaining two of Y₁, Y₂ and Y₄ are each N, and wherein Z is S.

31. The compound of claim 30, wherein Y₁ is CR, and Y₂ and Y₄ are N.

32. The compound of claim 30, wherein Y₂ is CR, and Y₁ and Y₄ are N.

33. The compound of claim 30, wherein Y₄ is CR, and Y₁ and Y₂ are N.

34. The compound of any one of claims 1-14, wherein the compound is of Formula (Ib) and Y₁, Y₂ and Y₄ are each CR.

35. The compound of claim 34, wherein each R is independently hydrogen, halogen, alkyl, cycloalkyl, haloalkyl, heteroaryl, —OR″, —N[C(O)R″]₂, —NR″C(O)R″, —NHC(O)OR″, —NHR″, —NH₂, or —NHSO₂R″CN;
optionally wherein each R″ is independently alkyl, cycloalkyl, aryl or benzyl.

36. The compound of claim 35, wherein:
L is hydrogen;
Z is 5;
R¹ is H;
T is C═O;
Y₁ is CH, C—OR″, CCl, C—CN, or C—NHC(O)R″;
Y₂ is CH, CCl, C-alkyl, C-cycloalkyl, or C-haloalkyl; and
Y₄ is CH, C—OR″, C—NHC(O)R″, C—NHC(O)OR″, C—NHR″, C—NH₂, or C—NHSO₂R″;
wherein, when Y₁ is CCl, then Y₂ is CH, C-alkyl, C-cycloalkyl, or C-haloalkyl;
optionally wherein each R″ is independently alkyl, cycloalkyl, aryl or benzyl.

37. The compound of claim 36, wherein:
Y₁ is CH;
Y₂ is CH or CCl; and
Y₄ is C—OR″ or C—NH₂, optionally C—OMe or C—NH₂.

38. The compound of any one of claims 1-35, wherein each R is independently hydrogen, halogen, alkyl, cycloalkyl, haloalkyl, heteroaryl, —NR″C(O)R″, NR″C(O)OR″, —NR″C(O)CH(OH)R″, —NHR″, —NH₂, —OR″, —CN, —C(O)NR″₂, or —NR″SO₂R″.

39. The compound of claim 38, wherein each R is independently hydrogen, halogen, alkyl, cycloalkyl, haloalkyl, —OR″, —CN, —NHC(O)R″, —NHC(O)OR″, —NHR″, —NH₂ or —NHSO₂R″.

40. The compound of claim 38 or 39, wherein each R″ is independently alkyl, cycloalkyl, aryl or benzyl.

41. The compound of any one of claims 1-3, wherein compound is of Formula (Ia), and wherein:
X₁ and X₂ are O
T is C═O
n is 1
R¹ is hydrogen
L is hydrogen, and
Z is S,
wherein,
when Y₁ and Y₃ are CR, then
(a) Y₂ is CH, Y₃ is CH, and Y₁ is C—NHC(O)R″;
(b) Y₁ is CH, Y₃ is CH, and Y₂ is C—OH or C—CH₂NHC(O)OR″; or
(c) Y₁ is CH, Y₂ is CH, and Y₃ is C—CH₂NHC(O)OR″; and
when Y₂ is N, then Y₃ is C—NHC(O)OR″ or C—NH₂.

42. The compound of any one of claims 1-3, wherein the compound is of Formula (Ib), and wherein:
X₁ and X₂ are O
T is C═O
n is 1
R¹ is hydrogen
L is hydrogen
Z is S, and
Y₂ and Y₄ are CR,
wherein:
when Y₁ is N, then Y₂ is CH, C—NH₂ or C—CH₂NHC(O)OR″ and Y₄ is CH or C—OR″, wherein at least one of Y₂ and Y₄ is not CH; and
when Y₁, Y₂ and Y₄ are each CR, then
Y₁ is CH, C-alkyl, C—Cl or C—OR″
Y₂ is CH, C-halogen, C-cycloalkyl, C-haloalkyl, C-aryl, C—CONH(R″), or C—CN
Y₄ is CH, C—NH₂, C—NHR″, C—NR″C(O)R″, C—NR″SO₂R″, or C—OR″;
at least one of Y₁, Y₂ and Y₄ is CH or C—Cl; and
(a) when Y₁ and Y₂ are CH, then Y₄ is C—NHCOR″, C—NHSO₂R″, C—OR″, or C—NH₂,
(b) when Y₄ is C—NHCOR″ and Y₁ is C—Cl, then Y₂ is C-cycloalkyl, and
(c) when Y₁ and Y₂ are CH and Y₄ is C—OMe, then the compound is:

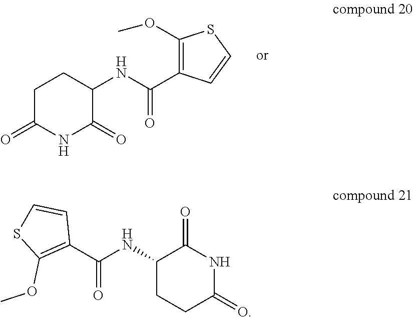 [SMILES: COc1sccc1C(=O)NC1CCC(=O)NC1=O ; COc1sccc1C(=O)N[C@H]1CCC(=O)NC1=O]

43. A compound of Formula (IIa) or (IIb): 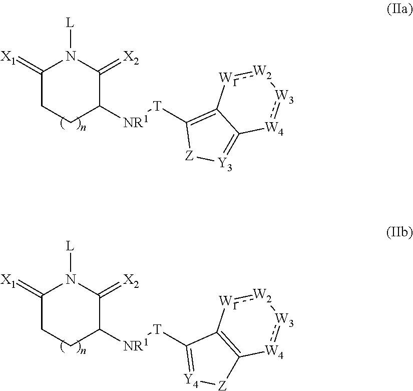
or a pharmaceutically acceptable salt or tautomer thereof,
wherein
each of X₁ and X₂ is independently O or S;
Z is O, S or NR²;
T is C═O or SO₂;
Y₃ is N or CR;
Y₄ is N or CR; [symbol]
indicates a single or double bond, wherein
when each [symbol] is a double bond, each of W₁, W₂, W₃ and W₄ is independently N or CR′, wherein at least one of W₁, W₂, W₃ and W₄ is N, and
when each [symbol] is a single bond, W₁, W₂, W₃ and W₄ are each CR′₂ and Y₄ is CR;
n is 0, 1 or 2;
L is hydrogen, alkyl, alkenyl, aryl, heteroaryl, benzyl, haloalkyl, haloalkenyl, —C(O)R″, —C(O)OR″, —C(O)NH₂, —C(O)NHR″, —C(O)NR″₂, —OR″, —NR″₂, or —S(O)₂R″;
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NR″C(O)R″, —NR″C(O)CH₂R″, —NR″C(O)CH(OH)R″, —NR″C(O)OR″, —NR″SO₂R″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, or —S(O)₂R″, —S(O)₂OR″, —S(O)₂NH₂, —S(O)₂NHR″, or —S(O)₂NR″₂;
each R′ is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NR″C(O)R″, —NR″C(O)CH(OH)R″, —NR″C(O)OR″, —NR″SO₂R″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, or —S(O)₂R″, —S(O)₂OR″, —S(O)₂NH₂, —S(O)₂NHR″, or —S(O)₂NR″₂;
each R″ is independently hydrogen, alkyl, cycloalkyl, heterocycloalkyl, alkenyl, cycloalkenyl, aryl, heteroaryl, or benzyl;
R² is hydrogen, alkyl, cycloalkyl, alkenyl, cycloalkenyl, alkynyl, aryl, heteroaryl, benzyl, haloalkyl, haloalkenyl, —NH₂, —NHR″, —NR″₂, —NR″C(O)R″, —N[C(O)R″]₂, —NR″C(O)OR″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, or —S(O)₂R″, —S(O)₂OR″, —S(O)₂NH₂, —S(O)₂NHR″, or —S(O)₂NR″₂; and
R¹ is hydrogen, alkyl, cycloalkyl, alkenyl, cycloalkenyl, aryl, heteroaryl, or benzyl;
wherein when each [symbol] is a double bond, Z is NR², R² is hydrogen, and each R′ is hydrogen, then W₄ is CR′.

44. The compound of claim 43, wherein the compound is of Formula (IIa).

45. The compound of claim 43, wherein the compound is of Formula (IIb).

46. The compound claim 43, having the structure:

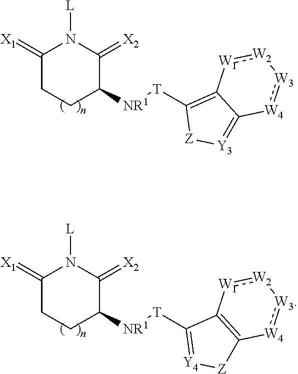

47. The compound claim 43, having the structure:

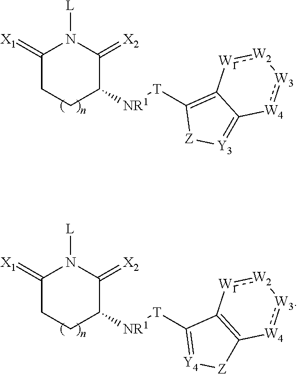

48. The compound of any one of claims 43-47, wherein when each [symbol] is a double bond, Z is NR², R² is hydrogen, and Y₃ and Y₄ are CR, one of W₁, W₂, W₃ and W₄ is N, and the remaining three of W₁, W₂, W₃ and W₄ are each CR′, then at least one R′ is not hydrogen

49. The compound of any one of claims 43-48, wherein Z is O.

50. The compound of any one of claims 43-48, wherein Z is S.

51. The compound of any one of claims 43-48, wherein Z is NR².

52. The compound of any one of claims 43-51, wherein T is C═O.

53. The compound of any one of claims 43-51, wherein T is SO₂.

54. The compound of any one of claims 43-53, wherein L is hydrogen, alkyl, alkenyl, aryl, heteroaryl, benzyl, haloalkyl, haloalkenyl, —C(O)R″, —C(O)OR″, —C(O)NH₂, —C(O)NHR″, —C(O)NR″₂, —NR″₂, or —S(O)₂R″; optionally wherein L is hydrogen, alkyl, alkenyl, aryl, heteroaryl, benzyl, —NR″₂, or —S(O)₂R″

55. The compound of any one of claims 43-53, wherein L is hydrogen, alkyl, alkenyl, aryl, heteroaryl, benzyl, —OR″, —NR″₂, or —S(O)₂R″; optionally wherein L is hydrogen, alkyl, alkenyl, aryl, heteroaryl, or benzyl.

56. The compound of claim 55, wherein L is hydrogen.

57. The compound of any one of claims 43-56, wherein Y₃ is N.

58. The compound of any one of claims 43-56, wherein Y₃ is CR.

59. The compound of any one of claims 43-56, wherein Y₄ is N.

60. The compound of any one of claims 43-56, wherein Y₄ is CR.

61. The compound of any one of claims 43-60, wherein each [symbol] is a double bond or wherein each [symbol] is a single bond.

62. The compound of claim 61, wherein each [symbol] is a double bond.

63. The compound of claim 62, wherein one of W₁, W₂, W₃ and W₄ is N, and the remaining three of W₁, W₂, W₃ and W₄ are each CR′.

64. The compound of claim 63, wherein one of W₁, W₂ and W₃ is N, and W₄ is CR′.

65. The compound of claim 62, wherein two of Ali, W₂, W₃ and W₄ is N, and the remaining two of W₁, W₂, W₃ and W₄ are each CR′.

66. The compound of claim 62, wherein one of W₁, W₂, W₃ and W₄ is CR′, and the remaining three of W₁, W₂, W₃ and W₄ are each N.

67. The compound of claim 61, wherein each [symbol] is a single bond.

68. The compound of any one of claims 43-67, wherein each R is independently hydrogen, halogen or —NR″C(O)R″.

69. The compound of any one of claims 43-68, wherein at least one R′ is not hydrogen.

70. The compound of any one of claims 43-68, wherein each R′ is hydrogen.

71. The compound of any one of claim 43, 44, 46 or 47, wherein the compound is of Formula (IIa), and wherein:
X₁ and X₂ are O
T is C═O
n is 1
R¹ is hydrogen
L is hydrogen
Z is S
each [symbol] is a double bond
W₁ is N
W₂, W₃ and W₄ are each independently CH or C—Cl; and
Y₃ is CH or C-halogen.

72. The compound of any one of claims 43 and 45-47, wherein the compound is of Formula (IIb), and wherein:
X₁ and X₂ are O
T is C═O
n is 1
R¹ is hydrogen
L is hydrogen
Z is NH
each [symbol] is a double bond
W₄ is N
W₃ is CH
one of W₁ and W₂ is C—Cl, and the other of W₁ and W₂ is CH; and
Y₄ is CH.

73. The compound of any preceding claim, wherein X₁ and X₂ are O.

74. The compound of any one of claims 1-70 wherein X₁ is O and X₂ is S.

75. The compound of any one of claims 1-70, wherein X₁ is S and X₂ is O.

76. The compound of any one of claims 1-70, wherein X₁ and X₂ are S.

77. The compound of any preceding claim, wherein n is 0.

78. The compound of any one of claims 1-76, wherein n is 1 or 2.

79. The compound of claim 78, wherein n is 1.

80. The compound of claim 78, wherein n is 2.

81. A compound of any one of the preceding claims, for use as a cereblon binder.

82. A pharmaceutical composition comprising a compound of any one of claims 1-80.

83. A compound of any one of claims 1-80, a composition according to claim 82, or a compound selected from 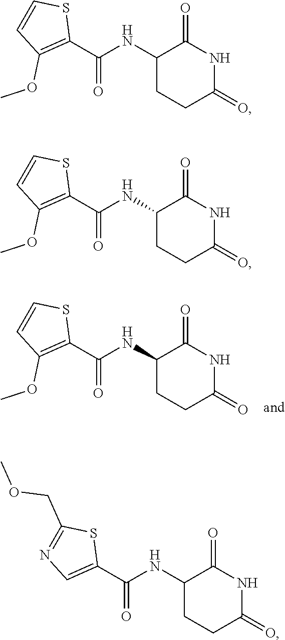 [SMILES: COc1ccsc1C(=O)NC1CCC(=O)NC1=O ; COc1ccsc1C(=O)N[C@H]1CCC(=O)NC1=O ; COc1ccsc1C(=O)N[C@@H]1CCC(=O)NC1=O ; COCc1ncc(C(=O)NC2CCC(=O)NC2=O)s1]
for use in medicine.

84. A compound of any one of claims 1-80, a composition according to claim 82, or a compound selected from 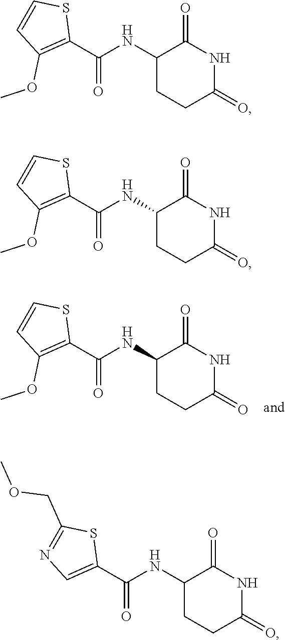 [SMILES: COc1ccsc1C(=O)NC1CCC(=O)NC1=O ; COc1ccsc1C(=O)N[C@H]1CCC(=O)NC1=O ; COc1ccsc1C(=O)N[C@@H]1CCC(=O)NC1=O ; COCc1ncc(C(=O)NC2CCC(=O)NC2=O)s1]
for use in immune-oncology.

85. A compound of any one of claims 1-80, a composition according to claim 82, or a compound selected from 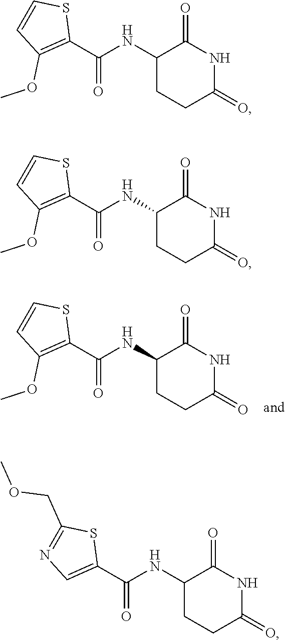 [SMILES: COc1ccsc1C(=O)NC1CCC(=O)NC1=O ; COc1ccsc1C(=O)N[C@H]1CCC(=O)NC1=O ; COc1ccsc1C(=O)N[C@@H]1CCC(=O)NC1=O ; COCc1ncc(C(=O)NC2CCC(=O)NC2=O)s1]
for use in the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders.

86. The compound or composition for use of any one of claims 83-84, wherein the compound is a compound of any one of claims 1-80.

87. A method for the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders;
wherein the method comprises administering to a patient in need thereof an effective amount of compound of any one of claims 1-80, a composition according to claim 82, or a compound selected

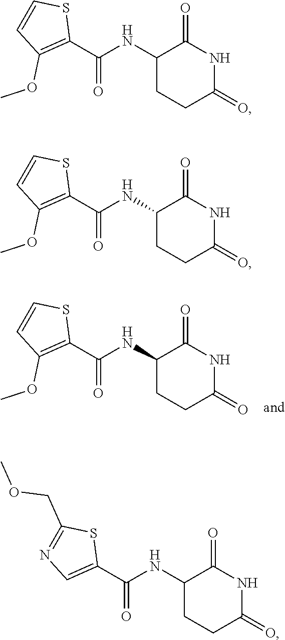 [SMILES: COc1ccsc1C(=O)NC1CCC(=O)NC1=O ; COc1ccsc1C(=O)N[C@H]1CCC(=O)NC1=O ; COc1ccsc1C(=O)N[C@@H]1CCC(=O)NC1=O ; COCc1ncc(C(=O)NC2CCC(=O)NC2=O)s1]

88. The method of claim 87, wherein an effective amount of compound of any one of claims 1-80, or a composition according to claim 82, is administered to the patient.

89. The method of any one of claims 87-88, further comprising administering at least one additional active agent to the patient.

90. A combined preparation of a compound of any one of claims 1-80 or a compound selected from 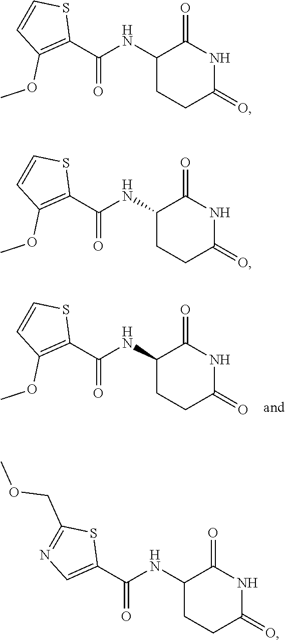 [SMILES: COc1ccsc1C(=O)NC1CCC(=O)NC1=O ; COc1ccsc1C(=O)N[C@H]1CCC(=O)NC1=O ; COc1ccsc1C(=O)N[C@@H]1CCC(=O)NC1=O ; COCc1ncc(C(=O)NC2CCC(=O)NC2=O)s1]
and at least one additional active agent, for simultaneous, separate or sequential use in therapy.

91. A combined preparation of a compound of any one of claims 1-80 and at least one additional active agent, for simultaneous, separate or sequential use in therapy.

92. The combined preparation of any one of claims 89-90, or the method of claim 89, wherein the at least one additional active agent is an anti-cancer agent or an agent for the treatment of an autoimmune disease.

93. The combined preparation of any one of claims 89-92, or the method of claim 89 or 92, wherein the at least one additional active agent is a small molecule, a peptide, an antibody, a corticosteroid, or a combination thereof.

94. The combined preparation or method of claim 93, wherein the at least one additional active agent is at least one of bortezomib, dexamethasone, and rituximab.

95. The combined preparation of any one of claims 90-94 wherein the therapy is the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders.
